(19) United States
(12) Patent Application Publication
(10) Pub. No.: US 2023/0309802 A1
(43) Pub. Date: Oct. 5, 2023

(54) ENDOSCOPE SYSTEM AND ASSEMBLIES

(71) Applicant: AdaptivEndo LLC, Louisville, KY (US)

(72) Inventors: Hershel E. Fancher, Georgetown, IN (US); Mark A. Griffin, Louisville, KY (US); Galen R. Powers, Louisville, KY (US); Trevor E. Powers, Louisville, KY (US); Michael W. Goldenbogen, Xenia, OH (US); Gregory R. Furnish, Louisville, KY (US); Nicholas Canfield, Louisville, KY (US); Nathan Wallace, Louisville, KY (US); Walter Stubblefield, Louisville, KY (US)

(73) Assignee: AdaptivEndo LLC, Louisville, KY (US)

(21) Appl. No.: 18/330,730

(22) Filed: Jun. 7, 2023

Related U.S. Application Data
(63) Continuation of application No. PCT/US2021/062210, filed on Dec. 7, 2021.
(60) Provisional application No. 63/260,209, filed on Aug. 12, 2021.
(60) Provisional application No. 63/203,552, filed on Jul. 27, 2021.
(60) Provisional application No. 63/203,619, filed on Jul. 27, 2021.
(60) Provisional application No. 63/225,682, filed on Jul. 26, 2021.
(60) Provisional application No. 63/186,484, filed on May 10, 2021.
(60) Provisional application No. 63/122,175, filed on Dec. 7, 2020.

(51) Int. Cl. A61B 1/00; A61B 1/005; A61B 1/015; A61B 1/018

(52) U.S. Cl. CPC A61B 1/0052; A61B 1/00105; A61B 1/015; A61B 1/018

(57) ABSTRACT

Endoscope assemblies, systems, and methods of manufacturing same are disclosed. In particular, endoscopes having controls and/or tool port openings one end of an elongate handle and the insertion tube assembly and umbilical extending to the console at another end of the elongate handle are provided. Fluid control and flow path arrangements in the handle of the endoscope are also described as well as configurations for articulation links and constructions for insertion tubes.

### CROSS-REFERENCE TO RELATED APPLICATION

This application is a continuation of PCT Application No. US2021/062210 filed Dec. 7, 2021, which is hereby incorporated by reference. PCT Application No. US2021/062210 claims the benefit of U.S. Provisional Application No. 63/122,175 filed Dec. 7, 2020, U.S. Provisional Application No. 63/186,484 filed May 10, 2021, U.S. Provisional Application No. 63/225,682 filed Jul. 26, 2021, U.S. Provisional Application No. 63/203,552 filed Jul. 27, 2021, U.S. Provisional Application No. 63/203,619 filed Jul. 27, 2021, and U.S. Provisional Application No. 63/260,209 filed Aug. 12, 2021, which are hereby incorporated by reference.

### BACKGROUND

This disclosure generally relates to endoscopes, including small diameter endoscopes including but not limited to cholangioscopes, ureteroscopes, cytoscopes, and/or hysteroscopes.

Endoscopes are used in a wide variety of medical procedures to visualize internal cavities or potential spaces within the human body during either diagnostic or therapeutic procedures. In particular, an endoscope for medical use has been widely used because it is possible to observe, without requiring dissection, a test target region in a body cavity by inserting an elongated insertion portion into the body cavity and perform curative treatment using a treatment instrument according to necessity.

Often, body cavities are so small that small diameter endoscopes are necessary to observe and treat within the cavity. For example, ureteroscopy is an examination of the ureter or kidney of a patient using an ureteroscope. Ureteroscopes typically include a long, thin, flexible portion that can be inserted through the patient’s urethra, bladder, and ureteral orifice connecting the bladder and the ureter. Ureteroscopes typically further include a camera and lighting system to adequately visualize the scope’s pathway and the working area within the ureter or kidney. Often, an ureteroscopy procedure involves treating a stone that has lodged in the patient’s mid to upper ureter or within a calyx of the kidney. Due to the size of the work space, it is desirable to produce scopes that are easily maneuverable within the small body cavities or tracts.

During endoscopic procedures, more than one scope may be utilized. This leads to overly complex systems that require more than one tool and multiple people assisting in the procedure. Due to the size of some operating theaters, it is desirable to decrease the complexity of these procedures and develop less complex, highly functioning scopes.

Thus, there is a need for improvement in this field.

### SUMMARY

The present disclosure pertains generally to endoscopes. It is understood that many of the aspects disclosed are applicable in other technical areas, but especially in other medical devices.

The present disclosure provides an improved endoscopes and associated consoles and cartridge assemblies. In some embodiments, the endoscopes may be small diameter endoscopes, such as ureteroscopes, cholangioscopes, cystoscopes, hysteroscopes, or bronchoscopes. The endoscopes may be a single use endoscopes and/or reusable use endoscopes. The endoscope assemblies attach to a cartridge by a tubing, sometimes referred to as an “umbilical”, extending from the endoscope assemblies to the cartridge that is connectable to consoles suitable for use with the endoscope assemblies.

The endoscopes include an insertion tube for insertion within the body of a patient. The insertion tube may include a distal end for insertion within the body of a patient and a proximal end. The insertion tube may include a main lumen extending through at least a portion of the insertion tube. The insertion tube may include an instrument tube extending within the interior of at least a portion of the insertion tube. The instrument tube preferably defines a tool channel allowing various instruments to be inserted and extended through the insertion tube and into a patient’s body.

The insertion tube may include an optical sensor module, such as a CCD and/or complementary metal oxide semiconductor (CMOS) image sensor, for providing information to the console and/or for projection of an image on a monitor such as a video feed from the end of the insertion tube. This video feed can be infrared, thermal or visible light. The insertion tube may include a light-emitting diode (LED) wire for providing power to an LED at the end of the insertion tube.

The endoscope may have a handle assembly with a handle body. The handle body may be composed of a first housing piece and a second housing piece. The handle assembly may include an insertion tube strain relief and an umbilical strain relief. The strain relief portions may help stabilize and support the connection between the rigid components of the endoscope with the insertion tube and umbilical. The strain relief portions may be internal and/or external to the handle body.

The handle body may be non-cylindrical in shape. For example, the handle body may have a pistol style shape. Advantageously, this shape can help improve the ability of an endoscopist to torque the insertion tube of the endoscope which can help steer the endoscope during advancement into and/or through the body of the patient and/or change the direction the camera of the endoscope is facing. A non-cylindrical handle may also provide a reference for the rotational orientation of the insertion tube.

The umbilical may be a consolidated umbilical incorporating both fluid lumens and electrical signal conductors extending between the cartridge and the endoscope’s handle assembly. Advantageously, a consolidated umbilical can reduce the number of tubes and/or cables that a user must manage during a procedure. Further, by consolidating the electrical signal conductors and fluid lumens within one umbilical attached to the console at a single location by the cartridge, a user is tethered to operating machinery/devices at only a single point. This arrangement can provide for more operating freedom.

An upper portion of the handle body may include an articulation control lever, a tool port and/or a fluid control button/trigger with a fluid flow adjustment mechanism. Advantageously, the articulation control lever may be located proximate the center of the handle body, facilitating both left-handed and right-handed use.

The tool port may be operatively positioned on the top of the upper portion of the endoscope. The tool port may also be centered on the endoscope similarly to the articulation control lever to promote ambidextrous use. The tool port is preferably an opening to the tool channel extending through the endoscope and to the distal end of the insertion tube, allowing a tool to pass through the endoscope and into a patient’s body. Advantageously, by locating the tool port on the top of the endoscope handle body, the excess portion of the tool that is not extended through the tool port and the tool channel may be less likely to impede an endoscopist’s movements compared to arrangements where the tool port is located on the side of the handle body. When the tool port is located on the side of the handle body, it may be more difficult for the endoscopist to grip the handle body, and the endoscopist may accidentally bump the tool. The location of the tool port preferably further allows the endoscopist to adjust the tool port using their thumb and forefinger of the same hand gripping the handle body. Such an arrangement can advantageously make use of the tool port less burdensome and allow for one-handed operation of the handle assembly of the endoscope. The tool port preferably includes a lock configured to lock in place a tool extending through the port.

The fluid control button preferably allows a user to control flow of irrigation fluid or suction from the endoscope’s handle using the forefinger of the same hand gripping the handle. Again, this preferably allows for one-handed operation of the handle assembly of the endoscope. Repeatedly pressing and releasing the flow control button generates a pulsatile flow. Pulsatile flow may be beneficial to dislodge an object located in a patient’s body, for example a urinary stone lodge in a patient’s ureter.

The fluid flow adjustment mechanism may be a lever or knob. In embodiments using a knob fluid flow adjustment mechanism, the knob may be rotated. Such rotation of the knob may move the fluid control button into an at least partially depressed state. Advantageously, the fluid flow adjustment mechanism may hold the fluid control button in the at least partially depressed state so as to provide a constant minimum irrigation or aspiration flow without requiring a user to apply continuous force to the fluid control button. If a higher flow rate is desired, the fluid control button may be depressed further from whatever the desired constant rate is all the way to a completely depressed and maximum flow rate. Additionally, a pulsatile flow may be used as described above. For example, the fluid flow adjustment mechanism may depress and hold the fluid control button to cause a twenty percent constant flow rate. If a higher flow rate or a pulsatile flow is desired, the user may further depress the fluid control button to receive a higher flow rate or pulsatile flow as described above. It should be appreciated that any desired constant flow rate may be achieved utilizing the fluid flow adjustment mechanism.

The endoscope assembly can include one or more pulley assemblies. Pulley assemblies can include at least one pulley operably arranged to rotate in response to movement of the articulation control lever. Associated with the at least one pulley is an articulation wire or a pair of articulation pull wires (e.g., two separate wires or one continuous wire looped over the pulley and having wire segments extending from opposing sides of the pulley) configured to bend a distal shaft section of the endoscope assembly upon actuation. The articulation wire may be fixed (e.g., tied or fixed by anchors to the pulley and/or extend around at least a portion of the pulley (e.g., loop around the pulley). The pulley may be a cam.

The articulation wire extends through the handle body and into and through the proximal portion of the insertion tube. Within the handle body, the articulation wires may extend through articulation wire management tubes.

In addition, the pulley assembly may include an articulation termination coil that receives one or more articulation coils wrapping around respective articulation wires connected to the pulleys. The articulation termination coil can provide an opposing force to the articulation wires. The articulation termination coil can be adjustable to remove articulation wire slack. For example, the articulation termination coil can be adjustable along the longitudinal axis of the endoscope assembly to control the articulation pull wire tension. The interior of the handle assembly may include a plurality of fixation slots for the proximal end of the insertion tube. Such arrangements can help maintain articulation wire tension during assembly of the endoscope. Fixation slots may be useful when the insertion tube is utilized to counteract the pull force of the articulation wires. Embodiments may utilize articulation coils and/or fixation slots.

Friction elements may be used to retain the articulation control lever position when released by the user. Advantageously, friction elements can keep the distal tip of the insertion tube from returning to a neutral configuration when the user releases the articulation control lever. Thus, for example, the user may be able to maintain the optical sensor module on a target viewing area without needing to maintain the position of the articulation control lever.

The second housing piece may extend through the one or more pulleys. The pulleys may be able to rotate around the second housing piece when rotated by the articulation control lever. Friction elements may be located between a static friction surface of the second housing piece and a rotating friction surface of the pulley. In some embodiments, friction grease may be used in place of the friction elements. In some examples, the friction elements may be O-rings. When the pulley is no longer being rotated to articulate the distal end of the insertion tube, the friction elements preferably maintain the pulley’s position and prevent the pulley from rotating back into a neutral position by resisting rotation of the rotating friction surface relative to the static friction surface.

The handle body may include a fluid pathway assembly. The fluid pathway assembly may be used for irrigation and/or suction. For illustrative purposes, the fluid pathway will be described in relation to irrigation.

The endoscope may include a first/primary irrigation lumen from a first irrigation source extending through the handle body. A valve incorporated within the handle body of the endoscope may be arranged to restrict and/or direct fluid flow within the first irrigation lumen. For example, the valve may be a pinch valve. The valve is preferably a normally closed valve such that during operation actuation of the fluid control button may actuate the valve on the first irrigation lumen so as to allow fluid to flow through the first irrigation lumen. The valve, however, may be a normally open valve. Advantageously, the user may control the valve using the thumb and/or forefinger of the same handle gripping the handle.

The first irrigation lumen may be attached to a first t-fitting. The first t-fitting may connect a first fluid path of the first irrigation lumen to the tool channel. A connecting lumen may extend from the first t-fitting towards the insertion tube. A second t-fitting may be included for integrating a second irrigation and/or suction source. The second t-fitting may be positioned along a fluid flow path between the first t-fitting and the insertion tube.

The endoscope may include a second/secondary irrigation lumen from the second irrigation source extending through the handle body. The second irrigation lumen is attached to the second t-fitting. The second t-fitting may be used to connect a second fluid path of the second irrigation lumen to the tool channel. The second t-fitting, first fluid path, second fluid path, and tool channel may all be in fluid communication. The second irrigation lumen arrangement may allow incorporation of a second irrigation/aspiration source without disrupting a user’s access to the first fluid control button controlled irrigation source. In one example, the first irrigation source can be a constant pressure bag, while the second source may be aspiration or a single action (pulsatile flow) irrigation pump. The second fluid source may be controlled remote from the handle assembly (e.g., by an assistant supporting the user during the procedure).

The second irrigation lumen preferably exits the handle through an umbilical strain relief and terminates with a connector, for example a female luer. The connector may be covered by a cap to prevent leakage when the primary irrigation source is in use and/or the insertion tube is inserted into a fluid filled cavity. While in use, the cap may be removed and a secondary fluid source may be connected to the connector, for example a fluid filled syringe.

The second irrigation lumen preferably exits the handle through the umbilical strain relief and terminates with a connector, for example a female luer. A syringe activated valve may be integrated with the connector to prevent leakage when the primary irrigation source is in use and/or the insertion tube is inserted into a fluid filled cavity. While in use, a syringe may be connected to the connector and valve to provide a secondary fluid source.

A one way valve may be located between the tool port and the first t-fitting. The one way valve may be incorporated in the first t-fitting. The one way valve may be a duck bill valve. The one way valve preferably resists unintended flow of fluid from the first irrigation source out of the tool port (e.g., an untightened tuohy borst adapter).

The insertion tube may have multiple sections including a proximal section, a transition section and an articulating section located at the distal end of the insertion tube. The proximal section of the insertion tube may be compromised of higher durometer polymers than the articulating section to resist bending of the proximal section during articulation of the articulating section. The articulating section may include an articulating link assembly (described in detail below) to articulate the distal end of the insertion tube. The articulating section may be made of low durometer polymers than the proximal section to allow the articulating section be bent by the articulating wires. The transition section is operably located between the articulating section and the proximal section. The transition section may be made of polymers with a high durometer measurement than the articulating section and a lower durometer measurement than the proximal section. This may allow the transition section to passively bend during articulation of the articulation section of the insertion tube. Therefore, when the articulating section is articulated there may be a smooth, fluid transition between the unmoving or minimally moving proximal section and the articulating section.

The compression coils may extend to and terminate at a proximal end of a proximal link in an articulating link assembly. The insertion tube may include an exterior polymer jacket and a braid on the interior of the jacket. Therefore, a single bonded insertion tube (which may include links, articulation wires, articulation coils, a camera assembly and electrical signal conductors) may be pulled through and seated in the handle assembly in a single action during assembly. This arrangement can ease assembly and repairs, while reducing labor costs.

The distal end may include a termination band. The articulation wires may be mounted against an interior wall of the termination band, for example by welding and/or an adhesive. The diameter of the termination band may be larger than the diameter of a cap assembly so that the cap assembly may be inserted into an opening of the termination band and secured thereto.

The insertion tube extends over the articulating link assembly. An electrical signal conductor extends through the articulating link assembly from the cap assembly from the camera and/or LED.

The articulating link assembly may include a plurality of links and/or support beams extending at least partially down a length of the plurality of links. The support beams may include breakaway support struts attached to the plurality of links. The plurality of links may be manufactured via an additive and/or substractive manufacturing process. The support beams and breakaway support struts may be used to support the links and keep the links aligned during the manufacturing process. Following assembly of the articulating link assembly, the support beams and support struts may be removed and discarded. The manufacturing process may allow for manufacturing a series of unique links (described in detail below). Preferably the manufacturing process does not require separate molds for each articulating link of the articulating link assembly.

The articulating link may include two openings for the articulation wires to extend through the link. The bottom of the link may include a concave/recessed articulating pivot and the top of the link may include a convex/protruding articulating pivot. Each link may include a hollow interior or lumen extending through the length of the link.

The plurality of links is formed of a series of two or more links. In some instances, the links are designed and arranged with progressively increasing angular separation between facing surfaces of adjacent links (i.e., angle X°) and/or decreasing length (L1, L2) along the length of the assembly (e.g., increasing angle X° and/or decreasing length along a direction from the proximal end to the distal end). Advantageously, such an arrangement may allow a user to bend the distal end more than the proximal end and/or bend the distal end before the proximal end starts bending.

The links may be modified in order to allow more articulation at the distal end of the articulation assembly. The links may be arranged to reduce the articulation wire force necessary to obtain the desired degree of articulation at the distal end of the insertion tube. For instance, links nearer the distal end may have shorter lengths than links nearer the proximal end. The links may also be arranged to increase the articulation angle at the distal end. The pivot radii of the protruding may be smaller for links nearer the distal end than the proximal end and/or the pivot radii of the recessed articulating pivots may be greater for links nearer the distal end than the proximal end.

All of these features can be modified on individual links or in combination with multiple links to achieve the desired articulation response and geometry. Advantageously, such arrangements can provide the ability to initiate articulation distally prior to or simultaneously with proximal articulation. Further, such arrangements can provide the ability to mitigate increased pull forces that accumulate at the proximal end of the articulation section. Finally, such arrangements can provide a uniform curvature of the radius along the articulation section from the proximal end to the distal end.

The plurality of links may be located throughout the articulating link assembly. The plurality of links may include a proximal termination link on the proximal end of the articulating link assembly and a distal termination link on the distal end of the articulating link assembly. The proximal termination link may be held laterally by the composite insertion tube. The distal termination link may be affixed to the camera assembly by the termination band. The articulation wires extending through the links may be laser welded to the termination band.

The distal end of the articulation wire may be mounted at the distal end of the insertion tube. The distal end of the articulation wire may extend through the distal termination link and/or may be laser welded to the termination band. The articulation wire may be laid within a groove, for example a U-shaped groove, in a stepdown portion of the distal termination link. The articulation wire may be secured, for example by an adhesive and locked into place by sliding the termination band over the stepdown portion of the distal termination link. The termination band preferably provides a secure connection between the distal termination link and the cap assembly.

Embodiments may not utilize articulating links. As described above, the insertion tube includes the proximal section, the transition section and the articulating section. The insertion tube may include an exterior polymer jacket and/or a braid on the interior of the jacket. Interior to the braid, a polytetrafluoroethylene (PTFE) sleeve may extend from the proximal section to the articulating section. The articulating wires may extend through the sleeve. The articulating wires may be secured to the walls of the termination band behind the braid, for example by welding. The articulating wires may be flat pull wires.

A main shaft PTFE liner may be positioned on a side of the PTFE sleeve opposite the braid. The liner may define the main lumen of the insertion tube. Advantageously, this arrangement may eliminate the need for links and/or increase the available area in the distal end of the insertion tube. Increased available cross-sectional area may facilitate the free movement of the tool channel, the electrical signal conductors and any other extrusions that may be located within the main lumen of the insertion tube. This arrangement may allow the distal end of the insertion tube to bend more than three hundred degrees and, in some arrangements, up to three hundred and ten degrees.

The umbilical assembly may include the umbilical and a first cartridge. The umbilical may include fluid lumens and electrical signal conductors extending between the cartridge and the endoscope’s hand assembly. The umbilical may be attached on one end of the umbilical to a cartridge at a position on the cartridge. The cartridge may include a housing. A second tube may extend from another location on the housing. The second tubing may be connected to the first irrigation source described above. The second tubing may include a Luer lock connector at one end of the tube to connect the second tubing to the first irrigation source. The second tubing may extend through the housing of the cartridge and into the umbilical. Advantageously, in case of a manual or mobile irrigation source, the second tube may reduce the chance of movement of the first irrigation source pulling the endoscope handle during a procedure.

The cartridge may include a printed circuit board (PCB). The PCB may include electrical signal spring tabs. The cartridge spring tabs may interface with the console to establish an electrical connection between the console, the cartridge and/or the endoscope. The cartridge may include a combination of static retention tabs and/or dynamic retention tabs. The retention tabs may operably connect the cartridge to a receiving receptacle of the console by connecting to corresponding retention slots. Retention tabs may provide for a “snap on” connection of the cartridge to the console. The ability to snap the cartridge on the console may allow for a quick and secure method for attaching the cartridge to the console or detaching the cartridge from the console.

The console may be relatively small so as to save space within the operating theater and on carts that may be in use. They console may not include fluid control. The console may include a receptacle. The receptacle may include an electrical signal connector to connect the electrical signal spring tabs of the first cartridge to the console. The receptacle may further include retention slots to receive and connect the dynamic retention tab of the cartridge and a retention slot for the static retention tab of the cartridge. This connection may create an electrical connection between the console and the cartridge. The dynamic retention tabs may be automatically released from the dynamic retention slots if an excessive amount of tension is placed on the umbilical. Advantageously, this safety release may prevent unintended damage to the console or cartridge. Only the static retention tabs and slots may be utilized. In some instances, only the dynamic retention tabs and slots may be utilized. In other instances, both the static and dynamic retention tabs and slots may be utilized.

The console may further include an illumination control. The illumination control may control the power to the LED at the distal end of the insertion tube. The illumination control may control the wavelength of the light emitted from the LED. The console may also include a power control to power the console on or off. For example, the power control may be a switch, knob or button.

The cartridge may include a bracket to attach the console to a pole in a vertical configuration. The receptacle may be oriented to allow the cartridge to be connected to the cartridge in the same configuration whether the console is horizontal or vertical. The receptacle may be rotated to a desired orientation based on the orientation of the console. Advantageously, this may allow the desired ergonomics and tubing arrangement to be maintained regardless of console orientation within the operating theater.

The present disclosure further provides a second cartridge and a second console. The second cartridge and second console may be larger than the first console and first cartridge due to the use of fluid control mechanisms with the second console and second cartridge. The system may include the cartridge inserted into a control valve assembly of the console. The cartridge can be connected to the endoscope by the umbilical. For instance, the cartridge may be connected to a single-use endoscope by one or more fluid tubes and/or electrical conductors.

The cartridge may be connected to support equipment. The support equipment may include an irrigation pump, an insufflator, and/or a vacuum pump (e.g., stand-alone or central). The cartridge may be positioned between the endoscope and the support equipment.

In some examples, the console may include a user interface (not shown) allowing the user to alter the various variables of the endoscope system. In one example, the user interface may be a touchscreen integrated into an electric visual display. In another example, the user interface could include a keyboard, mouse, trackball, and/or touch sensitive pointing device, etc.

The console may include a control valve assembly for receiving and actuating upon a cartridge. The console may provide electrical power, fluids and/or fluid pressure, vacuum pressure, and/or send and/or receive electrical signals with the endoscope assembly. The console may include a monitor (not shown) for visualizing signals received from the endoscope assembly such as a video feed from a camera at the distal end of the single-use shaft assembly.

The control valve assembly may be used to selectively actuate an actuatable portion of fluid paths of the cartridge. The control valve assembly may include actuators and a support structure comprising a first surface and a second surface.

The actuator may be a linear actuator or rotational actuator. The actuator may be a solenoid. Although as few as one actuator is appreciated by the applicant, in preferred examples the control valve assembly includes four actuators. The actuators are arranged to actuate valve portions of fluid paths in the cartridge to selectively open and/or close the fluid paths.

The valve portions of the fluid paths may comprise any acceptable valve. For example, the valve portion may comprise a petcock. The valve portion may also comprise a portion of tubing arranged for compression (e.g., a pinch valve) between the anvil on the first surface of the door and the actuator (e.g., solenoid) of the control valve assembly. The valve portions may serve as a valve for any number of fluid functions of the endoscope, such as a suction valve associated with actuator, a camera flush valve associated with actuator, an insufflation valve associated with actuator, and/or an insufflation vent valve associated with actuator.

The actuators of the control valve assembly may include electrical connectors to be received by the console and electrically connect the actuators to the console. When connected to the console, the console can provide electrical power and user inputted commands to the actuator to open and/or close the fluid pathways of the cartridge.

The cartridge may include an identifier readable and/or writable by the control valve assembly. The identifier may include information regarding the type of cartridge and/or usage information (e.g., whether the cartridge has been used before). Preferably, the control valve assembly is configured to read and process the identifier of the cartridge (e.g., to determine the type of cartridge and/or whether the cartridge has been used previously). The identifier may be readable and/or writable through an/the electrical connector of the cartridge and/or through other means (e.g., RFID, optics, and/or mechanical contact).

The first surface of the support structure may include the electrical connector for forming electrical connections to the electrical connector of the cartridge. The electrical connection between the control valve assembly of the console and the cartridge may allow the console to receive images and control data (valve/actuator and/or image data) passed from the endoscope through the cartridge to the console. The electrical connection may allow the console to supply power to the light-emitting diode (LED) of the endoscope assembly.

The cartridge can be a single-use cartridge. The single-use cartridge can negate the need for the use of permanent valve bodies contained within a reusable endoscope and/or the console. This advantageously eliminates the need to sterilize reusable valves after every use.

The cartridge has a housing. The housing can include a first side and a second side. The housing further comprises a first fluid side and a second fluid side allowing for fluid to travel in and out of the cartridge housing. The housing may define one or more fluid pathways, fluid connector, and/or electrical connectors. For example, cartridge housing can include electrical connector in electrical communication with an electrical conductor of the endoscope and optionally an optical sensor module that contains optical sensor calibration data and/or a unique identifier for the endoscope. The housing may define windows. In one example, the housing includes four windows. The windows may be defined by inner surfaces of the housing. The inner surface may create a generally circular opening in the housing. The circumference of one window may be larger than the other windows to receive a larger fluid path cross section.

The windows can be configured to receive an actuator from the console into a chamber defined by the inner surface. As described above, the actuator may pass through the window and/or contact an anvil on the door of the control valve assembly of the console to create a pinch valve with the door. When the cartridge is positioned on the control valve assembly and the door is closed, the windows align with the anvils on the door and the actuators of the control valve assembly. The windows may be arranged to define a geometric shape. For instance, the windows may define a diamond.

As described above, one or more actuators may be solenoids configured to extend into the windows of the cartridge housing and/or contact the anvils on the door of the control valve assembly creating a pinch valve when closed.

The housing may further include an electrical connector for connecting to the electrical connector of the control valve assembly. The electrical connector of the cartridge may be spring finger electrical contacts configured to contact a corresponding number of flat pad electrical contacts on the control valve assembly of the console or vice versa. The electrical connector of the cartridge housing may pass images and control data provided by the endoscope to the console. The electrical connector of the housing may pass power from the console to the LED used by the endoscope in the patient’s body.

The cartridge is inserted flush with the first surface of the support structure of the control valve assembly. The first fluid side of the cartridge may include loose tubing that continues into the umbilical and to the single-use shaft assembly of the endoscope (not shown).

The second fluid side of the cartridge may be connected to support equipment. The support equipment may include an irrigation pump, an insufflator, and/or a vacuum pump (stand-alone or central). The cartridge may provide for fluid communication between the endoscope and the support equipment.

The control valve assembly may include a ledge surface and a latch. The cartridge may include a ledge surface and a latch receiving portion. In alternate examples, the control valve assembly may include the latch receiving portion and the cartridge may include the latch. When the cartridge is received by the control valve assembly the latch contacts the latch receiving portion to secure a first end of the cartridge to the control valve assembly. Separately, and opposite of the latch and latch receiving portion, the ledge surface of the cartridge and the ledge surface of the control valve assembly may contact each other to secure a second end of the cartridge to the control valve assembly.

In some examples, the cartridge may include two or more magnets located on the first side of the cartridge. In some examples, one magnet may be located at the bottom of the first side of the cartridge and the second magnet located at the top of the first side of the cartridge. The first surface of the control valve assembly may include two or more magnetic sensors (hall or reed), mirroring the locating of the magnets on the cartridge. For example, one located at the bottom of the first surface and the other located at the top of the first surface. The bottom magnet on the cartridge may interface with the bottom magnetic sensor on the control valve assembly to trigger the actuator (e.g., solenoid) valves to retract to reduce the force required to tilt the cartridge into the control valve assembly. The top magnet may interface with the top magnetic sensor on the control valve assembly to confirm the presence of the cartridge on the console, triggering the console to release the appropriate actuators and initiate the actuator operation mode for endoscopy.

The system may include a first cartridge and a second cartridge. The first cartridge may be connected to a first endoscope (not shown) and the second cartridge may be connected to a second endoscope (not shown). The first cartridge and the second cartridge may be securely inserted into a first control valve assembly and a second control valve assembly of the console, respectively. The console may have any number control valve assemblies each capable of receiving an independent cartridge. The first control valve assembly may receive the first cartridge, and the second control valve assembly may receive the second cartridge. It may not be necessary for every control valve assembly to receive a cartridge during every medical procedure.

Each cartridge may be connected to a separate set of support equipment and an independent medical device, such as an endoscope.

The first control valve assembly and the second control valve assembly are configured to operate independently and/or in a main/secondary communication configuration. The first (aka “mother”) endoscope may be the main controller and the second (aka “daughter”) endoscope may be the secondary controller. In this arrangement, the controls of the main endoscope may control the fluid functions (e.g., image controls and/or LED) of one or more secondary endoscopes. The main endoscopes may control the function of the fluid paths of the secondary endoscopes by providing an electrical signal to the second control valve assembly to selectively actuate an actuator as described herein (e.g., through one or more cartridges connected to the console). The first endoscope may further control a function of the second endoscope (e.g., operation of the LED and/or camera) by providing an electrical signal from the first endoscope to alter a condition of the second control valve assembly (e.g., alter an electrical signal provided by the second control valve assembly to the cartridge of the second endoscope). For instance, the control valve assembly may provide an electrical signal to the LED and/or the camera of the second endoscope based on the signal received from the first endoscope. This arrangement further allows for multiple endoscopes to be setup and utilized in a procedure at once, eliminating or reducing extensive setup time typically used to change one endoscope out for another endoscope and/or eliminating or reducing the need for additional equipment, such as multiple control consoles.

The console may include a user interface. The user interface may be any suitable input/output device. The user interface may include a touchscreen.

The second control console may include fluid controls and may be larger than the console without fluid controls. The second console may include one or more receptacles/control valve assemblies. The receptacles may include an electrical signal connector to connect the electrical signal spring tabs of the cartridge to the console. The second console may allow a user to attach a cartridge with fluid control or without fluid control. In some examples, a large cartridge may be connected to the receptacle of the large second console. The larger second cartridge may cover a portion of the receptacle. In this example, as described in detail above, the console includes valves that may actuate to control fluid flow through the cartridge. In another example, the first cartridge described above may be secured to cover a smaller portion of the receptacle of the second console than the large cartridge. In a third example, a large cartridge may be used to cover the entire receptacle, but the fluid controls are not utilized.

Advantageously, for example, a cartridge without fluid control may provide the flexibility for connection to either a smaller pole mounted console such as one found in a medical office setting or to a larger console (e.g., a dual console) such as one found in an operating room setting for ureteroscopy. Further, a single-use cholangioscope may be used in conjunction with a hybrid duodenoscope during endoscopic retrograde cholangiopancreatography such as by connecting both to a dual console. Additionally, a single-use cholangioscope could be used with the smaller console, without fluid control, in conjunction with a third party reusable duodenoscope and its associated video processing unit.

An improved 4-way steering flexible endoscope is further disclosed. An endoscope assembly may include an umbilical cord and an insertion tube. The insertion tube may include an articulating portion. The insertion tube may further include articulation wires. The articulation wires are used to articulate the articulating portion of the insertion tube. In one embodiment, the insertion tube includes two articulation wires for either up-down or left-right articulation. In one exemplary embodiment, the insertion tube includes four articulation wires. This configuration allows for up-down and left-right articulation by the user.

Further disclosed is a mounting structure to mount one endoscope onto the handle of another endoscope. The ability to mount and secure one endoscope to the other allows the endoscopist to handle both endoscopes easier and decreases the cumbersome nature of the two endoscopes. Often, a small diameter endoscope is deployed through the tool channel of a larger diameter endoscope, for example a cholangioscope through a duodenoscope. The mounting structure is arranged to secure the small diameter endoscope to the handle and/or strain relief of the large diameter endoscope.

The mounting structure may include an elastic strap with an alignment pad, for example an elastomeric pad. In some embodiments, the alignment pad may be curved to match the typical shape of the body of a large diameter endoscope. The alignment pad prevents movement of the small diameter scope on the large diameter scope. The mounting mechanism may further include a cam latch and land for the cam latch. In the open position, the cam latch is free of the land for the cam latch. In a close/affixed position, the cam latch is secured to the land for the cam latch. In the closed position, the small diameter endoscope may be securely affixed to the body of a large diameter endoscope.

A variable integral suction control may be integrated into the handle of the endoscope. The suction control allows for variable manual suction actuation. Further, it allows for selection of a variable suction strengths. The valve could additionally control the flow of irrigation fluid.

The suction control may include an integral variable suction valve and a suction tube. The integral suction valve may inclyde a valve variable screw, a valve sliding button, a valve spring and a valve anvil. The screw may be rotatable or compressed without rotation. As the screw is rotated, the suction tube is either compressed against the valve anvil to lower the strength of suction or the compression against the valve anvil is released to increase suction. The level of suction may range from no suction, if the suction tube is completely shut off against the valve anvil, to completely open if the screw is not compressing the suction tube against the anvil. By adjusting the screw, a user is able to obtain a variable amount of suction.

The insertion tube may be a solid Polytetrafluoroethylene (PTFE) extrusion with integral lumens extending within the insertion tube (as described below). The insertion tube may include a lumen defining a tool channel for extending various medical devices through the insertion tube and into the body of a patient. The insertion tube may further include another lumen for one or more electrical conductors to pass an electrical signal to a camera and/or light diode at the distal end of the insertion tube, for example for a complementary metal oxide semiconductor (CMOS) sensor wire. The insertion tube may further include fluid tubes, such as an irrigation tube or a suction tube. As discussed above, the insertion tube may further include articulation wires to articulate the insertion tube. Finally, the insertion tube includes a core profile surrounded by a braid .

The core profile may be fabricated of a lubricious polymer incorporating a central lumen with four articulation wire lumens to extend the articulation wires through. In some embodiments, the central lumen is PTFE. In one exemplary embodiment, the articulation wire lumens are equally spaced around the circumference of the central lumen. The four lumens (lumen for the tool channel, the lumen for the electrical conductors and the two fluid lumens) may be made of nylon, EVA, and/or PVC.

The braid surrounds at least a portion of the exterior of the core profile’s central lumen. The braid may be encompassed by a sheath/outer jacket. The outer jacket may comprise HDPE, EVA, PVC, and/or PBAX.

During manufacture of the insertion tube, the lumen defining the tool channel, the lumen with the electrical conductors extending through them, various fluid tubes and articulation wire lumens may be pulled through the central profile’s central lumen. In one embodiment, the lumens are all pulled at once. This limits manufacturing method limits the number of leak points.

The articulating section may include two opposed series of articulation cuts in the core profile. These cuts are made throughout at least a portion of the length of the articulating section. These cuts create a series of articulating living hinges that promote bending in a single plane, for example at ninety degrees to one another. The articulating section may be fabricated from a different material from the core profile, for example nylon. The articulating section may be separably attached to the rest of the insertion tube via an adhesive, by a mechanical connection, or by virtue of the articulation wires extending through the articulating section and the rest of the insertion tube.

The insertion tube may have multiple sections including a proximal section, a transition section and an articulating section located at the distal end of the insertion tube. The proximal section of the insertion tube may be compromised of higher durometer polymers than the articulating section to prevent bending of the proximal section during articulation of the articulating section. The articulating section may be made of lower durometer polymers than the proximal section to allow the articulating section be bent by the articulating wires. The transition section is operably located between the articulating section and the proximal section. The transition section may be made of polymers with a higher durometer measurement than the articulating section and a lower durometer measurement than the proximal section. This allows the transition section to passively bend during articulation of the articulation section of the insertion tube. Therefore, when the articulating section is articulated there is a smooth, fluid transition between the unmoving or minimally moving proximal section and the articulating section.

The insertion tube may further include a camera cap assembly located at the distal end of the insertion tube. The insertion tube may further include a transition band between the articulating shaft section and the transition band. The diameter of the transition band may be larger than the diameter of the cap assembly so that the cap assembly may be inserted into an opening of the termination band and secured thereto.

An insertion tube termination fitting may receive articulation compression coils that receive one or more articulation coils wrapping around respective articulation wires. The articulation wires are configured to bend a distal shaft section of the endoscope assembly upon actuation. The insertion tube termination fitting further assists in securing the insertion tube to the endoscope handle body.

The camera cap assembly may include a camera cap, an optical sensor (camera) within the camera cap and a tool channel. The tool channel may extend from the endoscope handle body through the insertion tube and exits out of the camera cap.

The insertion tube includes an articulating section polymer jacket, a transition section polymer jacket and a proximal section polymer jacket encompassing the articulation section, transition section and proximal section, respectively. The three jackets compose an outer composite tube. The outer composite tube may be assembled in a single operation on a mandrel prior to insertion of any of the interior components of the insertion tube.

The insertion tube may include a plurality of articulating links extending throughout the insertion tube. The plurality of links are formed of a series of two or more links. The links facilitate articulating the distal portion of the insertion tube (articulating shaft section) within a patient’s body. The use of distal articulation links creates a greater degree of articulation based on a given input force when tools are in place within the tool channel as compared to other methods.

A coil and a heat stake attach the coil to the proximal shaft section. The insertion and heat staking of the coil in the proximal shaft section, secondary to the initial shaft layup simplifies fabrication.

The proximal shaft may include the outer proximal section polymer jacket. The polymer jacket may extend circumferentially around the proximal shaft section. A braided wire may extend within the polymer jacket. The coil may be within the braided wire.

The transitional shaft section may be identical to the proximal shaft section with the transition section polymer jacket instead of the proximal section polymer jacket.

The articulating shaft includes the outer articulating section polymer jacket. The polymer jacket may extend circumferentially around the articulating shaft section. A braided wire extends within the polymer jacket. The braided wire encircles the assembly of articulating links.

During manufacturing of the insertion tube, the articulation components (coils, articulation wires, links and internal tubing) may be pulled as a singular unit into the composite insertion tube. During manufacturing, the proximal shaft section including the compression coils and articulation wires may be extended through the articulating shaft section and pulled through the composite insertion tube until the proximal shaft section meets the insertion tube termination fitting and the compression coils and pulls wires extend past the insertion tube termination fitting. Pulling all the interior articulation components as a whole can make assembly it easier. Advantageously, the articulation coils can prevent unintended movements of the proximal shaft section by isolating articulation forces from the proximal shaft.

To mount the distal end of the articulation wire at the distal end of the insertion tube, the articulation wire may be positioned within a groove, for example a U-shaped groove, in a stepdown portion of a distal articulation link. The articulation wire may be secured, for example by an adhesive and locked into place by positioning of the transition band over the distal articulation link. This provides a simple method of fixation with a continuous wire, thus limiting the risk of a loose wire end.

In a second embodiment to mount the distal end of the articulation wire at the distal end of the insertion tube, the distal end of the articulation wire extends through the distal articulation link and is affixed (e.g., laser welded) to the transition band.

In both embodiments, the transition band provides a secure connection between the distal articulation link and the cap assembly. Advantageously, both embodiments allow for the fixation of the distal end of the articulation wire within the limited space of the small diameter endoscopes.

During manufacturing, a plurality of links or printed links may use support beams extending at least partially down a length of the plurality of links. The support beams include breakaway support struts attached to the plurality of links. The plurality of links may be formed during manufacturing of the plurality of links. As explained above, links of the plurality of links may different from one another. Such manufacturing may be injection molding, additive manufacturing, and/or subtractive manufacturing. The support beams and breakaway support struts may support the links and keep the links aligned during manufacturing. During assembly of the articulating link assembly, the support beams and support struts are removed and discarded.

A runner with breakaway support struts may be attached to the plurality of links. The runner may inclyde support struts, and the links may be formed by injection molding. The links may be connected by flexible link connectors. The flexible links connectors may be foldable after removal of the breakaway support struts and/or runner to pivot adjacent links of the articulating links towards one another. Advantageously, the flexible link connectors may aid in the proper ordering and orientation of the links during assembly.

The proximal (bottom) end of the articulation link may include two spaced apart concave/recessed articulating pivots and the distal (top) end of the link may include two corresponding convex/protruding articulating pivots. Each concave/recessed articulating pivot and its corresponding convex/protruding articulating pivot form a pair.

When articulated to one side, only one pair of the convex/concave articulating pivots may be in contact with one another. When articulated to a different side, a different pair of the convex/concave articulating pivots may be in contact with one another. Accordingly, when pivoted to one side, the adjacent links may articulate around a different pivot point than when the assembly is pivoted to a different side. In such instances, an articulation wire may be located a first distance from the pivot point when the assembly is articulated to a first side and a second distance when the assembly is articulated to a second side. Advantageously, such arrangements can allow an articulation wire to generate a greater moment (i.e., rotational force) for a given tensile force in the wire to pivot the adjacent links relative to one another when the assembly is articulated to one side verses another.

The plurality of links are formed of a series of two or more links. In some instances, the links are designed so that the distance between the pairs of convex/concave articulating pivots varies to control where articulation occurs first. By shortening the distance between the pivots about the centerline of the link, a given force may generate more articulation torque, as explained above. In a preferred embodiment, the distance between the convex/concave pivots decreases for successive pairs of convex/concave pivots along a direction toward the distal tip of the insertion tube. While articulating, an articulation angle is created between the links.

All of these features can be modified on individual links or in combination with multiple links to achieve the desired articulation response and geometry. Advantageously, this arrangement provides the ability to initiate articulation distally prior to or simultaneously with proximal articulation. Further, this arrangement provides the ability to mitigate increased pull forces that accumulate at the proximal end of the articulation section. Finally, this arrangement can provide a uniform curvature of the radius along the articulation section from the proximal end to the distal end.

Another embodiment of the insertion tube is disclosed herein. All applicable disclosure involving an insertion tube above is incorporated herein. The insertion tube may be composed of two sequential sections (proximal section and distal articulating section). The insertion tube may include a proximal multi-lumen shaft section with a multi lumen proximal extrusion and a deflection section illustrated as a steerable sheath. Between the two sections is a deflection transition section. The two sections are joined linearly by an exterior braid and multi-segmented polymer jacket.

The use of a proximal multi-lumen (including a central lumen and four articulation wire lumens as described above) shaft section simplifies the construction of the proximal shaft. The use of the central lumen facilitates pulling of the interior components, including the fluid lumens, lumen defining the tool channel, articulation wire lumens and electrical conductors as a single operation during assembly (as described above). The distal deflection section can be preassembled onto a mandrel along with the proximal multi-lumen extrusion prior to over-braid and reflow.

The insertion tube includes an outer proximal section polymer jacket, an inner deflection transition section liner, an outer deflection transition section jacket, an inner deflection section liner and an outer deflection section jacket.

One or more pairs of articulation wires may be bonded to the distal transition band. The transition band serves as a fixation point for the articulation wires and a transition to connect the distal tip of the deflection section with the camera cap assembly. The transition band provides a low-profile connection between the deflection section and the camera assembly cap. The transition band and articulation wire assembly can be inserted into the proximal/articulating composite tube thus facilitating assembly.

The deflection section is constructed as a steerable sheath. The steerable sheath construction may provide a larger internal lumen size relative to alternative steerable construction techniques.

The proximal shaft may include articulation wire lumens in which the articulation wires are extended. In some embodiments, there are four articulation wire lumens. The proximal shaft section may further includes a central lumen.

The deflection section further includes an articulation wire jacket.

During manufacturing of the insertion tube, the articulation components, including the articulation wires, may be pulled as a singular unit into the insertion tube. Additionally, during manufacturing, the proximal multi lumen shaft section including the articulation wires may be extended through the deflection section and pulled through the insertion tube until the articulation wires extend from the end of the insertion tube. By pulling all the interior articulation components as a whole, assembly is made easier.

A system for articulation wire tensioning with an adjustable stop is disclosed herein. The system moves either the compression coil or the catheter shaft distally relative to the proximal pull wire fixation point thus resulting in a tightening of one or more articulation wire.

The use of various systems to adjust the location of the articulation coil or insertion tube relative to the articulation wire are disclosed. Moving the articulation coil or insertion tube relative to the articulation wire provides the needed function of tensioning the articulation control assembly. Moving the articulation coil or insertion tube instead of adjusting the articulation wires has several advantages, including: 1) the articulation wire is more fragile than the articulation coil and, therefore, may be at greater risk of damage during tensioning/repositioning than the articulation coil; 2) if a crimp or adhesive is utilized to secure the articulation wire, it is more difficult to secure and to do so repeatedly than a cut coil/insertion tube; and 3) since the articulation wire typically terminates at a pulley, it is easier to incorporate the tension adjustment into the handle body where the articulating coil terminates.

The articulation compression coils may contact an adjustable stop. The stop may be arranged to counteract articulation wire force.

In another embodiment, the insertion tube may contact an adjustable insertion tube stop. To facilitate cooperation with the stop, the insertion tube may include an insertion tube collar.

In position 1, an adjustable coil spacer is positioned between the stop and a spacer retainer. The spacer retainer is utilized to keep the coil spacer in position against the stop. In a second position, the coil spacer is position such that the stop is between the coil spacer and the spacer retainer. In this configuration, the coil spacer prevents the compression coil from abutting against the stop. This configuration allows the insertion tube to be moved relative to the articulation wire and the handle body to tension the articulation control assembly.

A sliding coil retainer ma a compression be utilized to secure a coil. The compression coil may be secured within the coil retainer by any known mechanism. The coil retainer may include a plurality of grooves extending along the length of the coil retainer.

The handle body allows for selective repositioning of the coil retainer within the handle body for purposes of tensioning the articulation wires. The handle body may inclyde a plurality of grooves along the length of the handle body. As the coil retainer is selectively repositioned within the handle body, the grooves of the coil retainer and the grooves of the handle body may align. While in alignment, a pin may be inserted within the aligned grooves to lock the coil retainer and thus the compression coil in place. Other known locking mechanisms may be used to lock the coil retainer relative to the handle body, such as set screws and/or adhesive just to name a few nonlimiting examples.

Another embodiment to slide and retain the compression coil is disclosed herein. An articulation coil collar is disclosed with an upper portion wider than a lower portion of the collar. The collar receives and retains the compression coil. In some embodiments, the compression coil is inserted into and/or rotated into the coil collar. The handle body includes a plurality of grooves along the length of the handle body. The grooves are shaped to receive and retain the upper portion of the collar.

Yet another embodiment to slide and retain the compression coil is disclosed herein. The articulating wire may extend through an opening in a portion of the handle. A sliding stepped wedge may be position against the portion of the handle and covers the opening in which the wire extends through the portion of the handle. The sliding stepped wedge may include a stepped surface with varying heights. The compression coil may move between steps to change the amount of tensioning on the articulation wire.

Yet another embodiment to slide and retain the compression coil is disclosed herein. A rotating stepped wedge includes a stepped surface. In some embodiments, the rotating stepped wedge may include 3 steps of various heights. The compression coil may abut and may be secured against the stepped surface. Each stepped surface includes a groove for the articulation wire to extend through. As the rotating stepped wedge rotates, the compression coil may move between steps to change the amount of tensioning on the articulation wire 1008.

Another embodiment to alter the positioning of the compression coil in order to control tensioning of the articulation wire is disclosed herein. This embodiment includes a cannulated set screw configured against the handle. In this embodiment, the compression coil may be inserted into the set screw and rotated until it is securely retained by the set screw. The compression coil can be rotated within the set screw so that more or less of the compression coil is within the set screw depending on the amount of tensioning necessary.

Yet another embodiment to alter the positioning of the compression coil in order to control tensioning of the articulation wire is disclosed herein. This embodiment includes a rotating threaded hub configured to rotate within a plurality of grooves in the handle. The threaded hub may be rotated to move within the grooves. The compression coil may be inserted and retained within the rotating threaded hub. In order to alter tensioning on the articulation wire, the threaded hub is rotated clockwise or counterclockwise to alter the position of the compression coil in a proximal or distal direction.

A series of hybrid handle (single-use / reusable) variants associated with small diameter two direction (single plane) deflecting endoscopes is disclosed. These hybrid handle variants illustrate varying degrees of functionality associated with the single-use portion of the handle. For example, one hybrid handle variant requires remote fluid control, whereas another variant only incorporates electronic switches for image, illumination, etc. into the reusable handle. The two-direction hybrid handle variants disclosed herein support numerous clinical applications, including: cholangioscopy, ureteroscopy, cystoscopy, and bronchoscopy.

In addition to the hybrid handle variants disclosed, several two-way steering engagement mechanisms are disclosed. These engagement mechanisms involve both self-aligning shaft features and spring-loaded engagement features.

An endoscope assembly may include a separable reusable handle and a single use endoscope handle . The reusable handle may include electronic control switches and an electrical conductor. The electronic controls are for image capture, illumination power, video capture, etc.

The single use endoscope handle may include a tool port, an insertion tube, an umbilical, an articulation control lever, a fluid control button/trigger and an electrical conductors. The disclosure related to the tool port, the various insertion tubes, the umbilical, the articulation control lever and the fluid control button above is incorporated herein. The electrical conductor connects to another electrical conductor to pass electrical signals between the single use endoscope handle and the reusable handle. The electrical signal from the reusable handle may be passed to the cartridges described above and passed to a console ] when the cartridge \ is connected to the console. The electrical signals also pass from the consoles to the distal end of the insertion tube.

The use of the reusable handle reduces waste by moving portions of the endoscope handle structure, mechanism, and electronics away from a single-use, single piece endoscope to a reusable handle in a multi piece endoscope. The relocation of electronic controls from a single-use, single piece endoscope to a reusable handle of a multi piece endoscope, provides for the incorporation of additional features and higher quality components, while not burdening the single-use handle with additional costs. In other words, as opposed to a single use, single piece endoscope that must be thrown away after every use, more expensive and higher quality electronics may be used in the reusable handle and the single-use endoscope handle may be cheaper to manufacturer. Thus, lowering cost by allowing serial usage of the reusable handle with serial single use endoscope handles. The use of electrical conductors associated with the single-use endoscope handle to provide electrical power and signal connectivity to the reusable handle eliminates the need for secondary cables or batteries that need replacing or charging. The use of a wired connection also eliminates latency and/or connectivity issues often associated with a wireless connection.

Another embodiment of an endoscope assembly with a separable reusable handle and a single use endoscope handle is disclosed herein. Many components are similar to the embodiment disclosed above . Therefore, only the differences between the endoscope assemblies will be discussed.

The reusable handle includes electronic controls for image capture, illumination level, video capture, etc. and an articulation lever with optional brake. The reusable handle includes an articulation control lever, similar in function to the articulation control levers disclosed above, and an articulation drive shaft with engagement features. The single use endoscope handle includes an articulation pulley with engagement features configured to be compatible with articulation drive shaft. Movement of the lever will drive the drive shaft and in turn drive the pulley. The puller controls the articulation of the articulation wires within the insertion tube.

The use of a self-aligning detachable articulation drive (the drive shaft and the pulley) between the reusable handle and the single-use endoscope handle allows for the incorporation of additional features, such as a brake lever/mechanism, and higher quality components, while not burdening the single-use endoscope handle with additional costs. In other words, as opposed to a single use, single piece endoscope that must be thrown away after every use, more expensive and higher quality components may be used in the reusable handle and the single-use endoscope handle will be cheaper to manufacturer. Thus, lowering cost by allowing serial usage of the reusable handle with serial single use endoscope handles.

Another embodiment of an endoscope assembly with a separable reusable handle and a single use endoscope handle is disclosed herein.. Many components are similar to the embodiment disclosed above. Therefore, only the differences between the endoscope assemblies will be discussed herein.

The reusable handle includes electronic controls for image capture, illumination level, video capture, etc.; an articulation lever with optional brake; and a fluid button for mechanically pinching (controlling) a fluid conduit contained within the single use endoscope handle.

The single use endoscope handle includes a slot and a fluid control tube. When the reusable handle and single use endoscope handle are connected, a pinch valve is created between the fluid control button and the fluid control tube. The fluid control button may be pressed by a user to pinch the fluid control tube to limit the amount of fluid passing through the fluid control tube. The user may control the amount of fluid passing through the fluid control tube by pressing the fluid control button with various force. The fluid control button allows max flow through the tube when the button is not being pressed. The fluid through the tube decreases gradually in relation to the amount the user presses the control button inward.

This embodiment also relocates a mechanical pinch valve button and mechanism from the single-use endoscope handle to the reusable handle. This configuration allows for the incorporation of higher quality components, while not burdening the single-use cartridge with additional costs. In other words, as opposed to a single use, single piece endoscope that must be thrown away after every use, more expensive and higher quality components may be used in the reusable handle and the single-use endoscope handle will be cheaper to manufacturer. Thus, lowering cost by allowing serial usage of the reusable handle with serial single use endoscope handles.

Another embodiment of an endoscope assembly with a separable reusable handle and a single use endoscope handle is disclosed herein.. Many components are similar to the embodiment discussed above. Therefore, only the differences between the endoscope assemblies will be discussed herein.

The reusable handle includes electronic controls for image capture, illumination level, video capture, etc. and an articulation lever with optional brake. The reusable handle further includes an electrical switch for pinching (controlling) a fluid conduit using a remotely located electromechanical actuator.

The reusable handle includes an electrical switch. When a user presses the electrical switch, a signal is passed to an electromechanical actuator and the actuator actuates onto a fluid path limiting or preventing fluid flow through the fluid path (as described above).

This embodiment replaces the mechanical pinch valve button and mechanism with an electrical switch associated with the reusable handle and an electromechanical pinch valve located remote to the endoscope assembly. By removing the mechanical pinch valve from the single-use endoscope handle and/or reusable handle, the cost of the single-use cartridge is reduced.

A self-aligning articulation drive shaft and associated articulation pulley with engagement features are disclosed herein. As disclosed above, a reusable handle may include an articulation drive shaft. The drive shaft is driven by articulation control lever as described above.

The housing of the reusable handle supports the drive shaft connected to the articulation control lever. The drive shaft extends into the region of the reusable handle that receives the single use endoscope handle. The drive shaft of can extend into a recess of the single use endoscope handle such that, when the reusable handle and the single use endoscope handle are joined together, the drive shaft engage the pulley located in the single use endoscope handle.

The drive shaft may inclyde a pulley engaging portion. This portion may have a non-circular cross-sectional geometric shape such that it is capable of transmitting torque. This geometric shape can include, but is not limited to, oval, spline, square, or star, just to name a few non-limiting examples.

The single use endoscope handle can include one or more pulleys. The pulley can be arranged to receive an end of the drive shaft. Preferably, actuation of the articulation control lever associated with the drive shaft rotates the pulley. Preferably, the pulley has an engaging portion to mate with the engaging portion of the drive shaft for rotationally coupling the pulley and drive shaft to one another. Preferably, the engaging portions do not rely solely on friction between the drive shaft and pulley. Preferably the mating features include a geometric interference between the articulation shaft and pulley.

Associated with the at least one pulley is an articulation wire or a pair of articulation wires (e.g., two separate wires or one continuous wire looped over the pulley and having wire segments extending from opposing sides of the pulley) configured to bend the distal end of the insertion tube. The articulation wires may be fixed to the pulley and/or extend around at least a portion of the pulley (e.g., loop around the pulley). Preferably the articulation wires are configured to bend the distal end of the insertion tube in orthogonal planes.

An alternative embodiment to engage the drive shaft with the pulley with spring-loaded engagement features is disclosed herein. All applicable discuss from the prior embodiment is incorporated herein. The drive shaft may include a spring loaded engagement tab. The spring loaded engagement tab may include one or more spring loaded retractable blades. The spring loaded engagement tab may mate with an engagement feature on the pulley to operably lock the drive shaft with the pulley. The engagement feature may include one or more slots to receive the retractable blades of the spring loaded engagement tab.

The spring loaded retractable blades (with lead-in ramp) allow the user to simply press the two components together. When articulating the control lever through its range of motion, the retracted spring loaded blades will extend and engage the articulation pulley when they encounter the slots on the bore of the articulation pulley. This allows the user to attach the single use endoscope handle without pre-aligning the reusable handle and the single use endoscope handle.

Yet another embodiment is illustrated to mate drive shaft with pulley is disclosed herein. The reusable handle may include a drive disk. The drive disk may include a center alignment pin, for example an alignment boss with a lead-in chamfer, and one or more spring loaded retractable pins, for example a spring loaded torque pin with a lead-in chamfer, offset from the centerline of the disk to transmit torque. The articulation pulley may include mating features including a center alignment hole and an offset hole with a lead-in chamfer with a lead-in for torque pin.

The spring loaded retractable offset pins (with lead-in ramp) allow the user to simply press the two components together. When articulating the control lever through its range of motion, the retracted spring loaded pins will extend and engage the mating features on the face of the articulation pulley. This allows the user to attach the single use endoscope handle without pre-aligning the reusable handle and the single use endoscope handle.

Further disclosed herein is a novel braid pattern to achieve the mutual benefits of a coil and a braid for an endoscope’s insertion tube. A novel braid pattern that provides the torqueability associated with a traditional braid while incorporating a unique multi-filar aligned braid pattern in one or more of the helical paths to simulate the kink-resistance associated with a coil is disclosed. This novel improved braid pattern can be created using a traditional braider mechanism with carrier locations modified to generate this novel braid pattern. This novel braid pattern can improve the kink-resistance of small diameter endoscopes, including but not limited to: cholangioscopes, ureteroscopes, cystoscopes, and bronchoscopes.

One embodiment for a small diameter endoscope insertion tube is a single helical filar over a multi filar group, where the multi filars are all in the same direction. In some embodiments the multi filar group can vary from two to ten or greater. In one exemplary embodiment, the multi filar group is three filars.

The single helical filar combined with the multi filar group in the opposite direction provide the torqueability associated with a general 1 over 1 braid pattern or other variants thereof.

The multi filar group are aligned helical patterns, all in the same direction. This configuration creates a simulated coil to provide the insertion tube kink-resistance. The ability to generate a simulated coil during the braiding process reduces process steps, eliminates the need for additional equipment, and eliminates the cost of using a coil when producing an endoscope insertion tube.

Any of the inventive aspects and examples discussed herein may be used independently or in combination with each other. A single medical device may include all the aspects disclosed herein.

Other aspects, objectives, and advantages of the present disclosure will become more apparent from the following detailed description when taken in conjunction with the accompanying drawings. Further forms, objects, features, aspects, benefits, advantages, and examples of the present disclosure will become apparent from a detailed description and drawings provided herewith.

### BRIEF DESCRIPTION OF THE DRAWINGS

FIG. 1 illustrates an endoscope assembly and a cartridge.

FIG. 2A illustrates a right side view of the endoscope assembly of FIG. 1.

FIG. 2B illustrates a back side view of the endoscope assembly of FIG. 1.

FIG. 2C illustrates a left side view of the endoscope assembly of FIG. 1.

FIG. 2D illustrates a front side view of the endoscope assembly of FIG. 1.

FIG. 3 illustrates a perspective view of the endoscope assembly of FIG. 1.

FIG. 4 illustrates an upper portion of the endoscope assembly of FIG. 1.

FIG. 5 illustrates the endoscope assembly of FIG. 1 with a housing piece removed.

FIG. 6 illustrates a cross-sectional view of a pulley assembly, including friction elements of the endoscope assembly of FIG. 1.

FIG. 7 illustrates a cross-sectional view of another embodiment of the pulley assembly, including friction elements of the endoscope assembly of FIG. 1.

FIG. 8A illustrates a cross-sectional view of the endoscope assembly of FIG. 1.

FIGS. 8B-8H illustrate a second/secondary fluid lumen.

FIG. 9 illustrates a cross-sectional view of the upper portion of the endoscope assembly of FIG. 4.

FIG. 10 illustrates a side view of an insertion tube of the endoscope assembly of FIG. 1 with the hand assembly removed.

FIG. 11A illustrates a cross-sectional view of the insertion tube of FIG. 10.

FIG. 11B illustrates another cross-section view of the insertion tube of FIG. 10.

FIG. 11C illustrates a cross-sectional view of an articulation section of the insertion tube of FIG. 10.

FIG. 12 illustrates an exploded view of a termination band and cap assembly of the insertion tube of FIG. 10.

FIG. 13 illustrates a partially exploded view of the insertion tube of FIG. 10.

FIG. 14 illustrates a plurality of links of the insertion tube of FIG. 10.

FIG. 15A illustrates a bottom view of a single link of the plurality of links of FIG. 14.

FIG. 15B illustrates a top view of a single link of the plurality of links of FIG. 14.

FIG. 16 illustrates a side view of two links of the plurality of links of FIG. 14.

FIG. 17 illustrates a cross-sectional view of an articulating link assembly of the insertion tube of FIG. 10.

FIG. 18 illustrates an exploded view of a distal link and the termination cap.

FIG. 19 illustrates an exploded view of another embodiment of the distal link and the termination cap.

FIG. 20 illustrates a cross-sectional of the articulation section of the insertion tube of FIG. 10.

FIG. 21 illustrates the endoscope assembly and the cartridge.

FIG. 22 illustrates the endoscope and the cartridge.

FIG. 23 illustrates a perspective view of the cartridge of the FIG. 22.

FIG. 24 illustrates an exploded view of the cartridge of FIG. 23.

FIG. 25 illustrates a perspective view a console and the cartridge.

FIG. 26A illustrates a perspective view of the console of FIG. 25.

FIG. 26B illustrates a perspective view of the console of FIG. 25 in a vertical orientation.

FIG. 27 illustrates a perspective view of another example of a cartridge and a console including a control valve assembly

FIG. 28 illustrates a perspective view of the control valve assembly of FIG. 27.

FIG. 29 illustrates a perspective view of the console of FIG. 27 and the cartridge from FIG. 23.

FIG. 30 illustrates a front perspective view of another endoscope assembly.

FIG. 31 illustrates a rear perspective view of the endoscope assembly of FIG. 30.

FIG. 32A illustrates a rear perspective view of an upper portion of the endoscope assembly of FIG. 30 with the mounting structure in the open position.

FIG. 32B illustrates a rear perspective view of an upper portion of the endoscope assembly of FIG. 30 with the mounting structure in the affixed position.

FIG. 33 illustrates a partially exploded view of the endoscope assembly of FIG. 30 with a top cover and pulleys removed.

FIG. 34 illustrates a partially exploded view of the endoscope assembly of FIG. 33.

FIG. 35 illustrates a cross-sectional view of an insertion tube.

FIG. 36 illustrates a side view of the articulating section of the insertion tube.

FIG. 37 illustrates a perspective view of an insertion tube with a handle assembly removed.

FIG. 38 illustrates a side view of the insertion tube of FIG. 37.

FIG. 39 illustrates a cross-sectional view of the insertion tube of FIG. 37.

FIG. 40A illustrates a cross-sectional view of a proximal shaft section of the insertion tube of FIG. 37.

FIG. 40B illustrates a cross-sectional view of a transition shaft section of the insertion tube of FIG. 37.

FIG. 40C illustrates a cross-sectional view of an articulating shaft section of the insertion tube of FIG. 37.

FIG. 41 illustrates a partially exploded view of the insertion tube of FIG. 37.

FIG. 42A illustrates a cross-sectional view of a distal end of the insertion tube of FIG. 37.

FIG. 42B illustrates a cross-sectional view of another embodiment of a distal end of the insertion tube of FIG. 37.

FIGS. 43A-B illustrates a plurality of links with support beams and support struts during the manufacture of the plurality of links.

FIG. 44 illustrates a plurality of links with flexible link connectors during the manufacture of the plurality of links.

FIG. 45 illustrates a plurality of links with flexible link connectors folded to attach a pair of articulating links together during the manufacture of the plurality of links.

FIG. 46 illustrates a side view of the plurality of links with compression coils and articulating links of the insertion tube of FIG. 37.

FIGS. 47A-B illustrates two assembled proximal articulating links.

FIGS. 48A-B illustrates two assembled distal articulating links.

FIG. 49 illustrates a perspective view of another embodiment of an insertion tube with a handle assembly removed.

FIG. 50 illustrates a side view of the insertion tube of FIG. 49.

FIG. 51 illustrates a cross-sectional view of the insertion tube of FIG. 49.

FIG. 52A illustrates a cross-sectional view of a proximal shaft section of the insertion tube of FIG. 49.

FIG. 52B illustrates a cross-sectional view of a deflection section of the insertion tube of FIG. 49.

FIG. 52C illustrates a cross-sectional view of a deflection transition section of the insertion tube of FIG. 37.

FIG. 53 illustrates a partially exploded view of the insertion tube of FIG. 49.

FIG. 54A illustrates the distal end of the insertion tube of FIG. 49 in a straight configuration.

FIG. 54B illustrates the distal end of the insertion tube of FIG. 49 in an articulated configuration.

FIG. 55A illustrates an insertion tube with compression coils against an adjustable stop.

FIG. 55B illustrates an insertion tube against an adjustable stop.

FIGS. 56A-B illustrates an embodiment of the adjustable stop in a first position and second position, respectively.

FIGS. 57A-B illustrates another embodiment of the adjustable stop in a first position and second position, respectively.

FIGS. 58A-B illustrates another embodiment of the adjustable stop in a first position and second position, respectively.

FIGS. 59A-B illustrates another embodiment of the adjustable stop in a first position and second position, respectively.

FIGS. 60A-B illustrates another embodiment of the adjustable stop in a first position and second position, respectively.

FIGS. 61A-B illustrates another embodiment of the adjustable stop in a first position and second position, respectively.

FIGS. 62A-B illustrates another embodiment of the adjustable stop in a first position and second position, respectively.

FIG. 63A illustrates a rear, perspective, exploded view of an endoscope with a reusable handle incorporating only electronic controls.

FIG. 63B illustrates a front, perspective, exploded view of the endoscope of FIG. 63A.

FIG. 63C illustrates another front, perspective, exploded view of the endoscope of FIG. 63A.

FIG. 64A illustrates a rear, perspective, exploded view of an endoscope with a reusable handle incorporating electronic controls and an articulation control lever.

FIG. 64B illustrates a front, perspective, exploded view of the endoscope of FIG. 64A.

FIG. 64C illustrates another front, perspective, exploded view of the endoscope of FIG. 64A.

FIG. 65A illustrates a rear, perspective, exploded view of an endoscope with a reusable handle incorporating electronic controls, an articulation control lever, and a mechanical push button for controlling a fluid conduit within a single use endoscope handle.

FIG. 65B illustrates a front, perspective, exploded view of the endoscope of FIG. 65A.

FIG. 65C illustrates another front, perspective, exploded view of the endoscope of FIG. 65A.

FIG. 66A illustrates a rear, perspective, exploded view of an endoscope with a reusable handle incorporating electronic controls, an articulation control lever, and an electronic switch for controlling a fluid conduit within a single use endoscope handle using a remotely located electromechanical actuator.

FIG. 66B illustrates a front, perspective, exploded view of the endoscope of FIG. 66A.

FIG. 66C illustrates another front, perspective, exploded view of the endoscope of FIG. 66A.

FIG. 67 illustrates an endoscope with a drive shaft and pulley with self-aligning engagement features.

FIG. 68A illustrates a cross-sectional view of the drive shaft and pulley of FIG. 67.

FIG. 68B illustrates a rear view of the drives shaft and pulley of FIG. 67.

FIG. 69 illustrates an endoscope with a drive shaft and pulley with spring-loaded engagement features.

FIG. 70A illustrates a cross-sectional view of the drive shaft and pulley of FIG. 69.

FIG. 70B illustrates a rear view of the drives shaft and pulley of FIG. 69.

FIG. 71 illustrates another embodiment of an endoscope with a drive shaft and pulley.

FIG. 72A illustrates a cross-sectional view of the drive shaft and pulley of FIG. 71.

FIG. 72B illustrates a rear view of the drives shaft and pulley of FIG. 71.

FIG. 73 illustrates a perspective view of a braid pattern for an insertion tube.

FIG. 74 illustrates a side view of the pattern of FIG. 73.

FIG. 75 illustrates an enlarged view of the pattern of FIG. 73.

FIG. 76 illustrates another enlarged view of the pattern of FIG. 73.

### DESCRIPTION OF THE SELECTED EMBODIMENTS

For the purpose of promoting an understanding of the principles of the disclosure, reference will now be made to the embodiments illustrated in the drawings and specific language will be used to describe the same. It will nevertheless be understood that no limitation of the scope of the disclosure is thereby intended. Any alterations and further modifications in the described embodiments, and any further applications of the principles of the disclosure as described herein are contemplated as would normally occur to one skilled in the art to which the disclosure relates. Exemplary embodiments of the disclosure are shown in great detail, although it will be apparent to those skilled in the relevant art that some features that are not relevant to the present disclosure may not be shown for the sake of clarity.

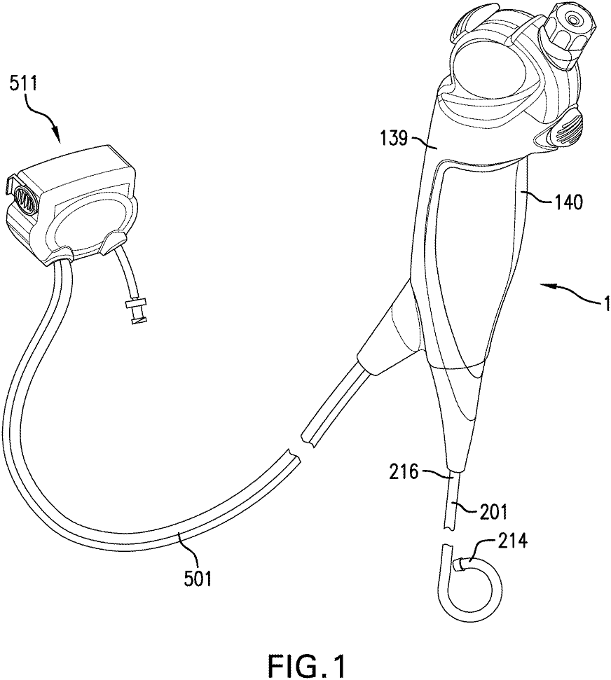

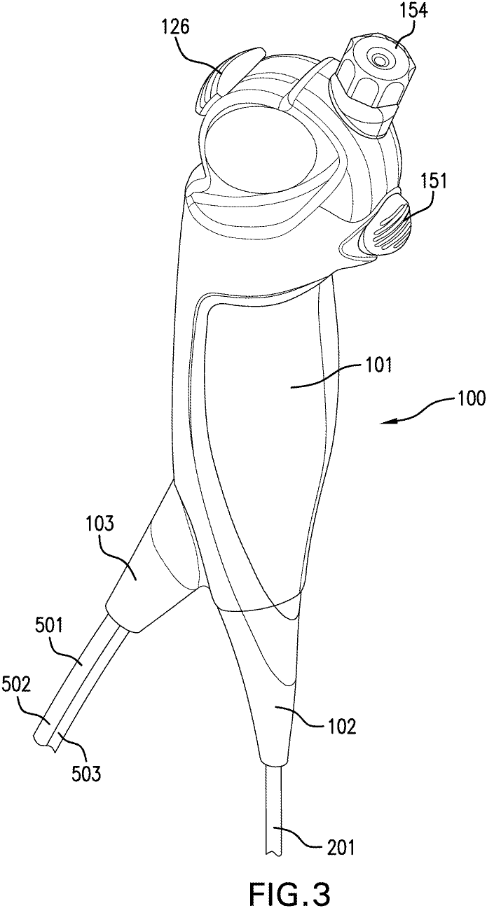

The present disclosure provides an improved endoscope and associated console and cartridge assembly. In some embodiments, the endoscope may be a small diameter endoscope, for example ureteroscopes, cholangioscopes, cystoscopes, hysteroscopes, or bronchoscopes. The endoscopes may be a single use endoscope or reusable use endoscope. FIGS. 1-4 illustrate various views of an endoscope assembly 1. FIG. 1 illustrates the endoscope assembly attached to a cartridge 511 by a tubing 501, sometimes referred to as an “umbilical”, extending from the endoscope assembly to the cartridge that is connectable to a console 600 or 700 suitable for use with the endoscope assembly.

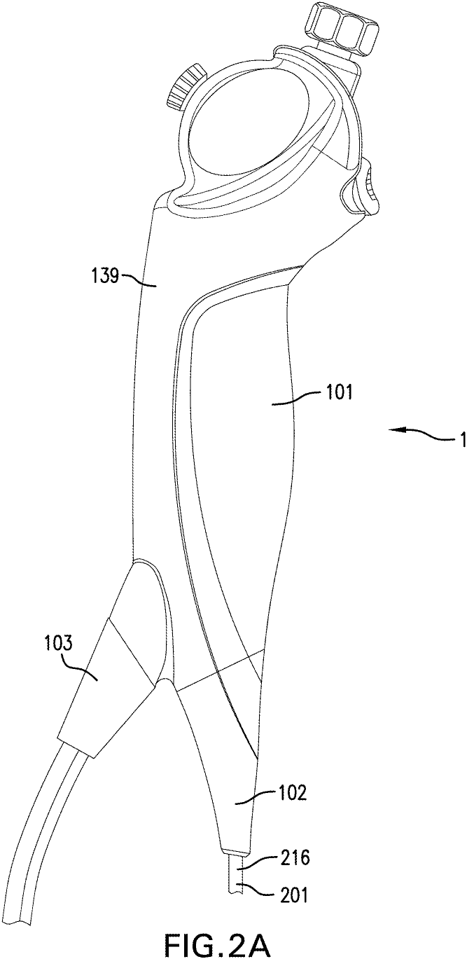

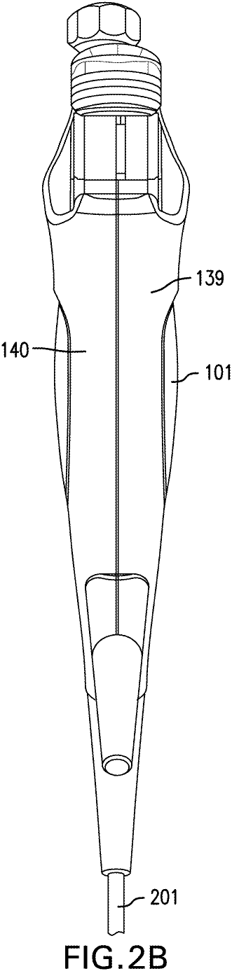

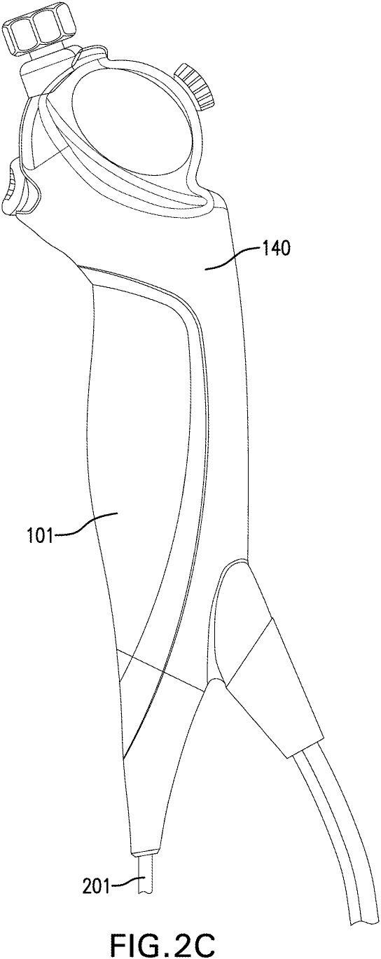

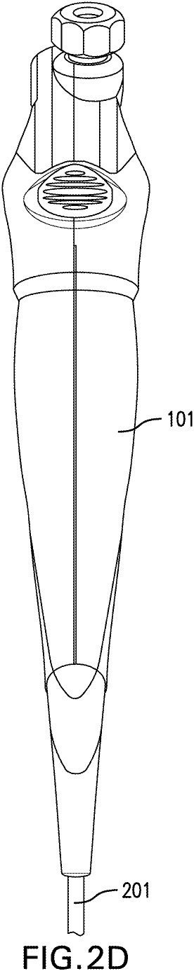

FIGS. 2a-d and 3 illustrate the endoscope including an insertion tube 201 for insertion within the body of a patient. The insertion tube may include a distal end 214 for insertion within the body of a patient and a proximal end 216. The insertion tube may include a main lumen 218 extending through at least a portion of the insertion tube. The insertion tube may include an instrument tube 162 extending within the interior of at least a portion of the insertion tube. The instrument tube defines a tool channel 161 allowing various instruments to be inserted and extended through the insertion tube and into a patient’s body.

The insertion tube may include an optical sensor module, such as a CCD and/or CMOS image sensor, for providing readable information by the console and/or project an image on a monitor such as a video feed from the end of the insertion tube. This video feed can be infrared, thermal or visible light. The insertion tube may include a light-emitting diode (LED) wire for providing power to an LED at the end of the insertion tube.

The endoscope is illustrated having a handle assembly 100 with a handle body 101. The handle body may be composed of a first housing piece 139 and a second housing piece 140. The handle assembly may include an insertion tube strain relief 102 and an umbilical strain relief 103. The strain relief portions may help stabilize and support the connection between the rigid components of the endoscope with the insertion tube and umbilical. In other exemplary embodiments, the strain relief is inverted within the interior of the handle body.

The handle body may be a pistol style shape as opposed to the typical cylinder shape. Advantageously, this shape can help improve the ability of an endoscopist to torque the insertion tube of the endoscope which can help steer the endoscope during advancement into and/or through the body of the patient and/or change the direction the camera of the endoscope is facing. A non-cylindrical handle may also provide a reference for the rotational orientation of the insertion tube.

FIGS. 1-3 illustrate the umbilical 501 as a consolidated umbilical incorporating both fluid lumens 502 and electrical signal conductors 202 within an electrical conductor extrusion 503 extending between the cartridge and the endoscope’s handle assembly. Advantageously, a consolidated umbilical reduces the number of tubes and/or cables that a user must manage during a procedure. In many instances, the electrical signal conductors and fluid lumens are run through separate tubing or umbilicals resulting in more clutter in the operating space. Further, by consolidating the electrical signal conductors and fluid lumens within one umbilical and attached to the console at a single location by the cartridge, a user is tethered to operating machinery/devices at only a single point. This arrangement provides for more operating freedom.

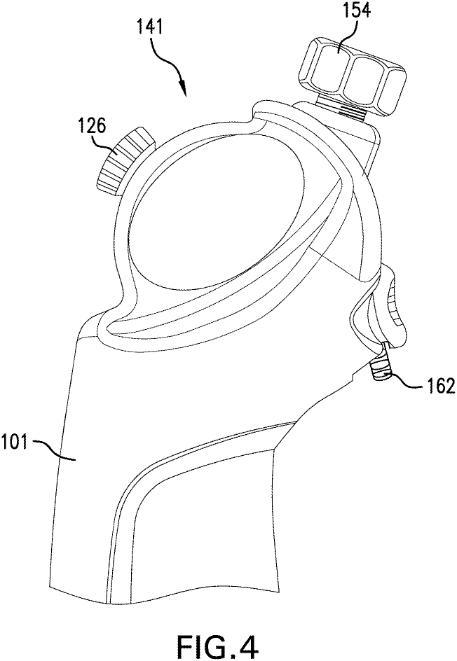

FIG. 4 illustrates an upper portion 141 of the handle body. The upper portion may include an articulation control lever 126, a tool port 154 and a fluid control button/trigger 151 with a fluid flow adjustment mechanism 162. Advantageously, the articulation control lever is mounted to the center of the handle body, facilitating both left-handed and right-handed use.

The tool port is operatively positioned on the top of the upper portion of the endoscope. The tool port may also be centered on the endoscope similarly to the articulation control lever to promote ambidextrous use. The tool port is an opening to the tool channel extending through the endoscope and to the distal end of the insertion tube, allowing a tool to pass through the endoscope and into a patient’s body. Advantageously, by locating the tool port on the top of the endoscope handle body, the excess portion of the tool that is not extended through the tool port and the tool channel is less likely to impede an endoscopist’s movements than arrangements where the tool port is located on the side of the handle body. When the tool port is located on the side of the handle body, it may be more difficult for the endoscopist to grip the handle body, and the endoscopist may accidentally bump the tool. The location of the tool port further allows the endoscopist to adjust the tool port using their thumb and forefinger of the same hand gripping the handle body, thus, use of the tool port is less burdensome and leaves one of the endoscopist’s hands free. The tool port is used to lock a tool being extended through the port in to place.

The fluid control button, in contrast to the typical remote commands necessary for irrigation, allows a user to control flow of irrigation fluid or suction from the endoscope’s handle using the forefinger of the same hand gripping the handle. Again, this allows the endoscopist to have a free hand to perform other tasks during the procedure. Repeatedly pressing and releasing the flow control button generates a pulsatile flow. Often, pulsatile flow is beneficial to dislodge an object located in a patient’s body, for example a urinary stone lodge in a patient’s ureter.

The fluid flow adjustment mechanism may be a lever or knob. In embodiments using a knob fluid flow adjustment mechanism, the knob may be rotated. Such rotation of the knob may move the fluid control button into an at least partially depressed state. Advantageously, the fluid flow adjustment mechanism may hold the fluid control button in the at least partially depressed state so as to provide a constant minimum irrigation or aspiration flow without requiring a user to apply force to the fluid control button. If a higher flow rate is desired, the fluid control button may be depressed further from whatever the desired constant rate is all the way to a completely depressed and maximum flow rate. Additionally, a pulsatile flow may be used as described above. For example, the fluid flow adjustment mechanism may depress and hold the fluid control button to cause a twenty percent constant flow rate. If a higher flow rate or a pulsatile flow is desired, the user may further depress the fluid control button to receive a higher flow rate or pulsatile flow as described above. It should be appreciated that any desired constant flow rate may be achieved utilizing the fluid flow adjustment mechanism.

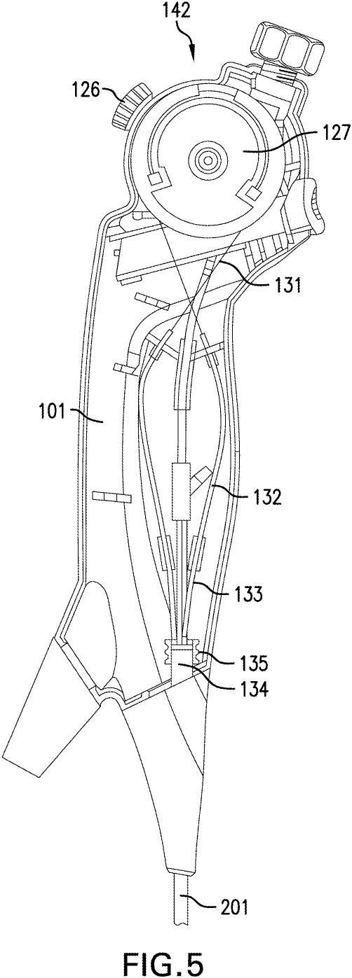

Regarding FIGS. 5-7, the endoscope assembly can include one or more pulley assemblies 142. Pulley assemblies can include at least one pulley 127 operably arranged to rotate in response to movement of the articulation control lever. Associated with the at least one pulley is an articulation wire 131 or a pair of articulation wires (e.g., two separate wires or one continuous wire looped over the pulley and having wire segments extending from opposing sides of the pulley) configured to bend a distal shaft section of the endoscope assembly upon actuation. The articulation wire may be fixed (e.g., tied or fixed by anchors (not illustrated) to the pulley and/or extend around at least a portion of the pulley (e.g., loop around the pulley). The pulley may be a cam.

The articulation wire extends through the handle body and into and through the proximal portion of the insertion tube. Within the handle body, the articulation wires extend through articulation wire management tubes.

In addition, the pulley assembly may include an articulation termination coil 133 that receives one or more articulation coils wrapping around respective articulation wires connected to the pulleys. The articulation termination coil provides an opposing force to the articulation wires. The articulation termination coil is adjustable to remove articulation wire slack. In other words, the articulation termination coil is adjustable along the longitudinal axis of the endoscope assembly to control the articulation pull wire tension. In other embodiments, the interior of the handle assembly may include a plurality of fixation slots 135 for the proximal end of the insertion tube. This arrangements helps maintain articulation wire tension during assembly of the endoscope. The fixation slots are useful when the insertion tube is utilized to counteract the pull force of the articulation wires. Some embodiments utilize the articulation coils and others may use the fixation slots. In other embodiments, both the fixation slots and articulation coils may be present. An insertion tube termination fitting 134 may be present to receive the articulation wires.

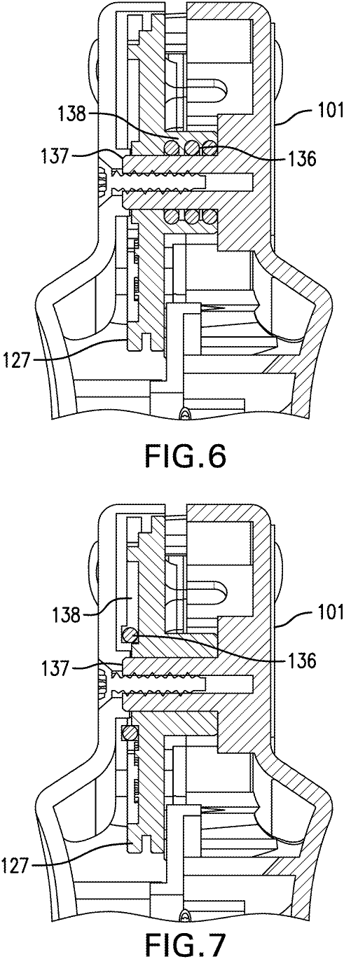

Turning to FIGS. 6 and 7, friction elements 136 for retaining the articulation control lever position when released by the user are illustrated. Advantageously, these friction elements keep the distal tip of the insertion tube from returning to a neutral configuration when the user releases the articulation control lever. Thus, the user is able to maintain the optical sensor module on a target viewing area without needing to maintain the position of the articulation control lever.

The second housing piece may extend through the one or more pulleys. The pulleys are able to rotate around the second housing piece when rotated by the articulation control lever. Friction elements may be located between a static friction surface 137 of the second housing piece and a rotating friction surface 138 of the pulley. In some embodiments, friction grease may be used in place of the friction elements. In some examples, the friction elements may be O-rings. When the pulley is no longer being rotated to articulate the distal end of the insertion tube, the friction elements maintain the pulley’s position and prevent the pulleys from rotating back into a neutral position by resisting rotation of the rotating friction surface relative to the static friction surface.

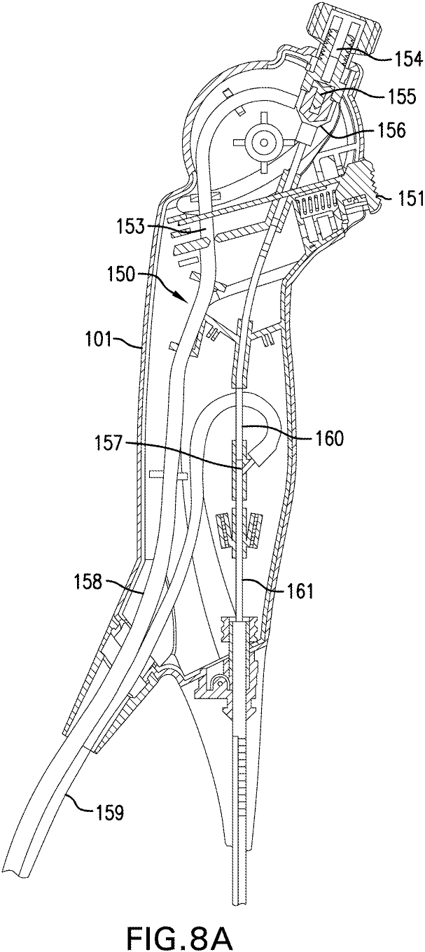

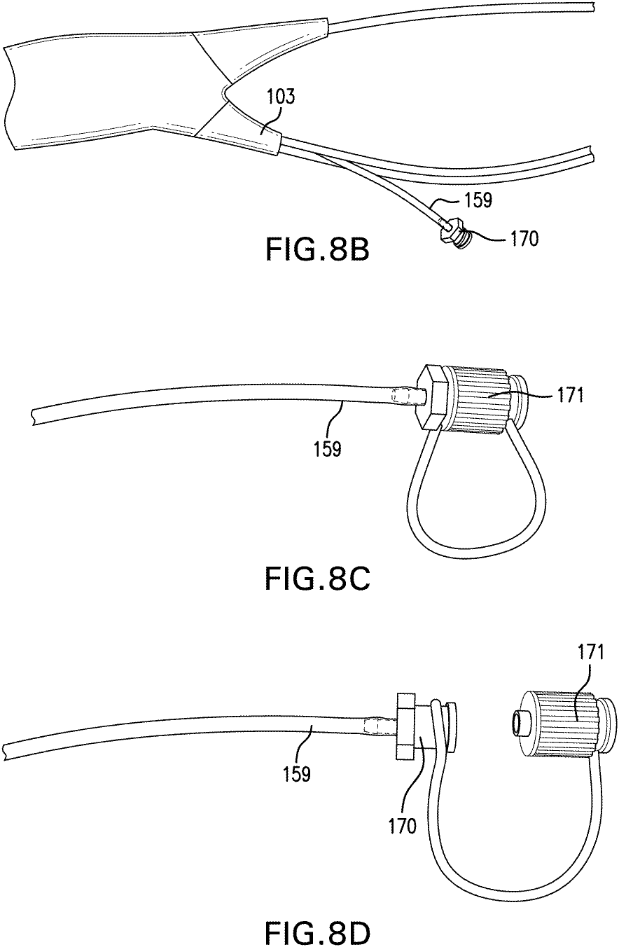

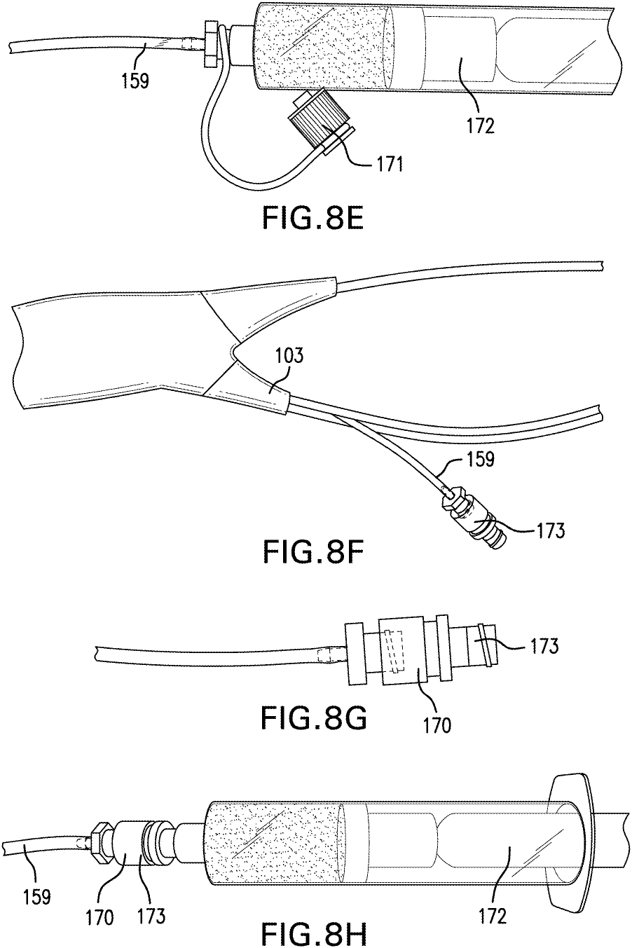

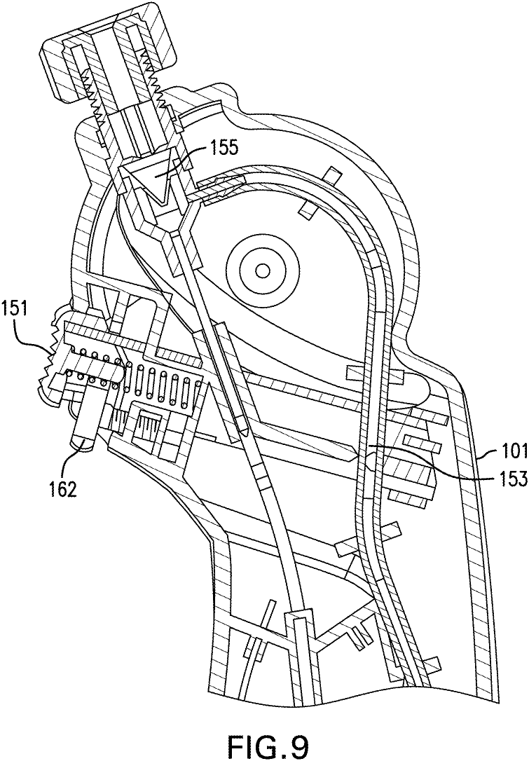

Turning to FIGS. 8-9, a fluid pathway assembly 150 within the handle body is illustrated. It is understood that the fluid pathway assembly may be used for irrigation and/or suction. For illustrative purposes only, the fluid pathway will be described in relation the irrigation.

The endoscope may include a first/primary irrigation lumen 158 from a first irrigation source (not shown) extending through the handle body. A valve 153 incorporated within the handle body of the endoscope may be arranged to restrict and/or direct fluid flow within the first irrigation lumen, for example the valve may be a pinch valve. The valve is preferably a normally closed valve such that during operation actuation of the fluid control button may actuate the valve on the first irrigation lumen so as to allow fluid to flow through the first irrigation lumen. The valve, however, may be a normally open valve. Advantageously, the user may control the valve using the thumb and forefinger of the same handle gripping the handle.

The first irrigation lumen is attached to a first t-fitting 156. The first t-fitting is used to connect a first fluid path of the first irrigation lumen to the tool channel. A connecting lumen 160 extends from the first t-fitting towards the insertion tube. A second t-fitting 157 may be included for integrating a second irrigation and/or suction source (not shown). The second t-fitting may be positioned along the fluid flow path between the first t-fitting and the insertion tube.

The endoscope may include a second/secondary irrigation lumen 159 from the second irrigation source (not shown) extending through the handle body. The second irrigation lumen is attached to the second t-fitting. The second t-fitting is used to connect a second fluid path of the second irrigation lumen to the tool channel. Thus, after the second t-fitting the first fluid path, second fluid path and tool channel are all in fluid communication. The second irrigation lumen arrangement allows incorporation of a second irrigation/aspiration source without disrupting user access to the first fluid control button controlled irrigation source. In one example, the first irrigation source can be a constant pressure bag, while the second source may be aspiration or a single action (pulsatile flow) irrigation pump. Often, the second fluid source may be controlled by an assistant supporting the user during the procedure.

FIGS. 8B-8E illustrate the second/secondary irrigation lumen in further detail. The second irrigation lumen exits the handle through the umbilical strain relief and terminating with a connector 170, for example a female luer. The connector may be covered by a cap 171 to prevent leakage when the primary irrigation source is in use or the insertion tube is inserted into a fluid filled cavity. While in use, the cap may be removed and a secondary fluid source may be connected to the connector, for example a fluid filled syringe 172.

FIGS. 8F-8H illustrate a second embodiment of the second/secondary irrigation lumen. The second irrigation lumen exits the handle through the umbilical strain relief and terminating with a connector 170, for example a female luer. A syringe activated valve 173 may be integrated with the connector to prevent leakage when the primary irrigation source is in use or the insertion tube is inserted into a fluid filled cavity. While in use, a syringe may be connected to the connector and valve to provide a secondary fluid source.

Further illustrated in FIGS. 8 and 9 is a one way valve 155 located between the tool port and the first t-fitting. The one way valve may be incorporated in the first t-fitting. In some preferred examples, the one way valve is a duck bill valve. The one way valve resists unintended flow of fluid from the first irrigation source out of the tool port (e.g., an untightened tuohy borst adapter).

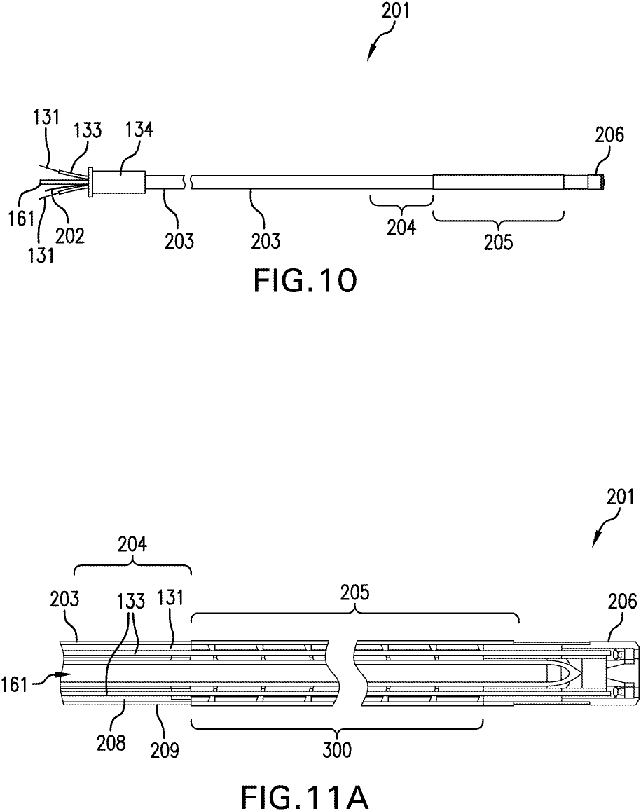

Turning to FIGS. 10-13, the insertion tube 201 is illustrated. The insertion tube may have multiple sections including a proximal section 203, a transition section 204 and an articulating section 205 located at the distal end of the insertion tube. The proximal section of the insertion tube may be compromised of higher durometer polymers to prevent bending of the proximal section during articulation of the articulating section. The articulating section may include an articulating link assembly 300 (described in detail below) to articulate the distal end of the insertion tube. The articulating section may be made of low durometer polymers to allow the articulating section be bent by the articulating wires. The transition section is operably located between the articulating section and the proximal section. The transition section may be made of polymers with a high durometer measurement than the articulating section and a lower durometer measurement than the proximal section. This allows the transition section to passively bend during articulation of the articulation section of the insertion tube. Therefore, when the articulating section is articulated there is a smooth, fluid transition between the unmoving or minimally moving proximal section and the articulating section.

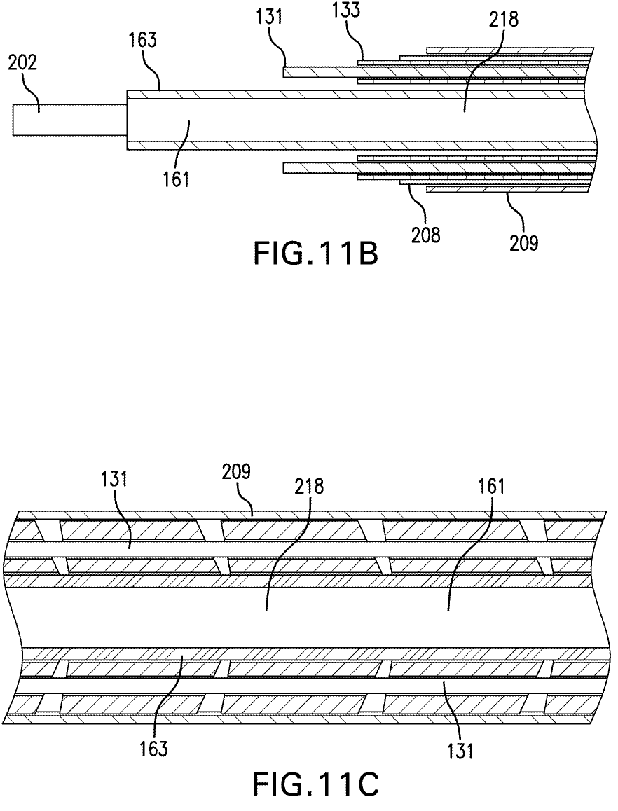

FIG. 11A further illustrates the articulation compression coils extend to and terminate at a proximal end of a proximal link in an articulating link assembly. FIGS. 11B and 11C illustrate a cross section of the insertion tube and a cross section of the articulation section in more detail. As demonstrated in the figures, the insertion tube includes an exterior polymer jacket 209 and a braid 208 on the interior of the jacket. Therefore, a single bonded insertion tube (which may include links, articulation wires, articulation coils, a camera assembly and electrical signal conductors) may be pulled through and seated in the handle assembly in a single action during assembly. This arrangement facilitates assembly and repairs, while reducing labor costs.

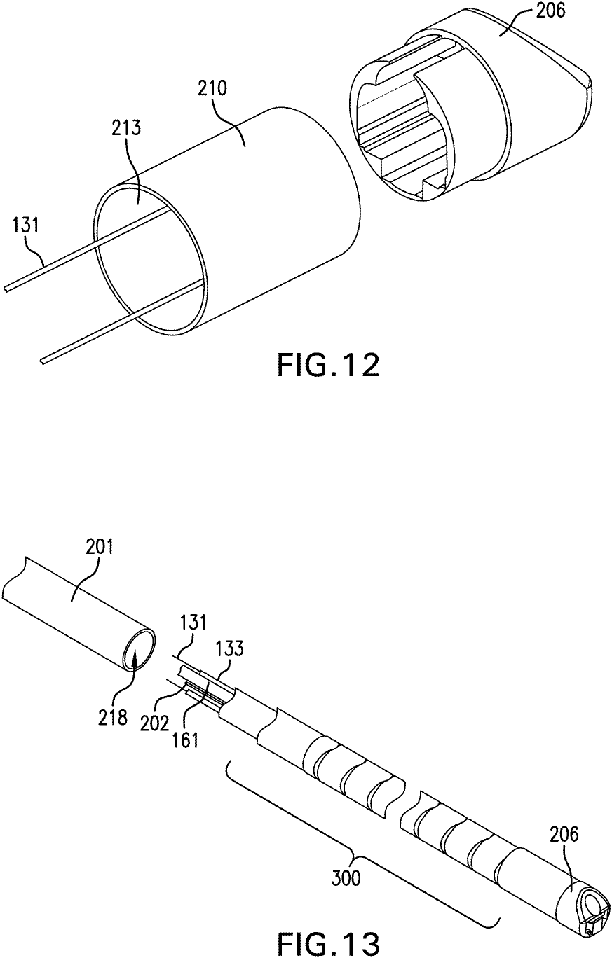

FIG. 12 illustrates the distal end of the insertion tube in greater detail. The distal end includes a termination band 210. The articulation wires may be mounted against an interior wall 213 of the termination band, for example by welding and/or an adhesive. The diameter of the termination band is larger than the diameter of a cap assembly 206 so that the cap assembly may be inserted into an opening of the termination band and secured thereto.

FIG. 13 demonstrates a partially exploded view of the assembly of the insertion tube over the articulating link assembly, including the electrical signal conductor 202 from the cap assembly for the camera and LED extending through the articulating link assembly.

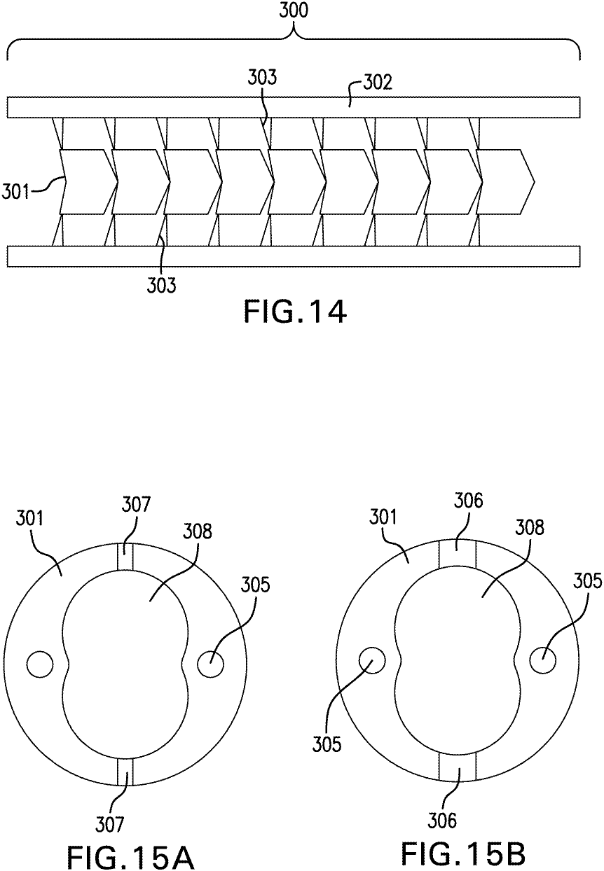

Turning to FIGS. 14 and 19, the articulating link assembly is shown in greater detail. FIG. 14 illustrates the articulating link assembly including a plurality of links 301 and support beams 302 extending at least partially down a length of the plurality of links. The support beams include breakaway support struts 303 attached to the plurality of links. The plurality of links may be manufactured via an additive manufacturing process. The support beams and breakaway support struts are used to support the links and keep the links aligned during the manufacturing process. Following assembly of the articulating link assembly, the support beams and support struts are removed and discarded. The additive manufacturing process allows for manufacturing a series of unique links (described in detail below) that would otherwise be very difficult and/or expensive to manufacture using traditional molding techniques.

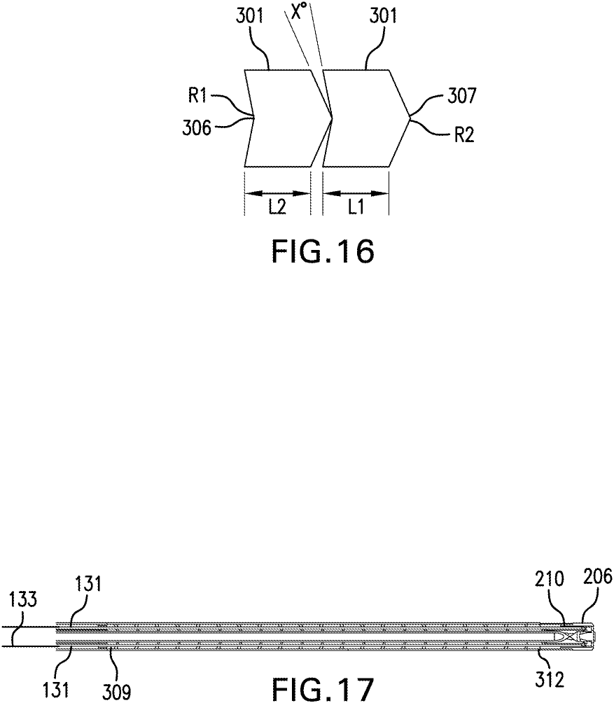

FIGS. 15A and 15B illustrate a bottom (FIG. 15A) and top (FIG. 15B) view of an articulating link 301. The articulating link may include two openings 305 for the articulation wires to extend through the link. As described in more detail with FIG. 16, the bottom of the link includes a concave/recessed articulating pivot 306 and the top of the link includes a convex/protruding articulating pivot 307. Each link further includes a hollow interior or lumen 308 extending through the length of the link 301.

FIG. 16 illustrates two assembled links 301. The plurality of links are formed of a series of two or more links. In some instances, the links are designed and arranged with progressively increasing angular separation between facing surfaces of adj acent links (i.e., angle X°) and/or decreasing length (L1, L2) along the length of the assembly (e.g., increasing angle X° and/or decreasing length along a direction from the proximal end to the distal end). Advantageously, such an arrangement may allow a user to bend the distal end more than the proximal end and/or bend the distal end before the proximal end starts bending. A radius R1 of the concave/recessed articulating pivot 306 may increase and/or a radius R2 of the convex/protruding articulating pivot 307 may decrease along a length of the articulation section.

Typically, the links are modified in order to allow more articulation at the distal end of the articulation assembly. The links may be arranged to reduce the articulation wire force necessary to obtain the desired degree of articulation at the distal end of the insertion tube. For instance, links nearer the distal end may have shorter lengths than links nearer the proximal end. The links may also be arranged to increase the articulation angle at the distal end. The pivot radii of the protruding may be smaller for links nearer the distal end than the proximal end and/or the pivot radii of the recessed articulating pivots may be greater for links nearer the distal end than the proximal end.

All of these features can be modified on individual links or in combination with multiple links to achieve the desired articulation response and geometry. Advantageously, this arrangement provides the ability to initiate articulation distally prior to or simultaneously with proximal articulation. Further, this arrangement provides the ability to mitigate increased pull forces that accumulate at the proximal end of the articulation section. Finally, this arrangement can provide a uniform curvature of the radius along the articulation section from the proximal end to the distal end.

FIG. 17 illustrates a cross sectional view of the articulating link assembly. The plurality of links are shown throughout the articulating link assembly. The plurality of links includes a proximal termination link 309 on the proximal end of the articulating link assembly and a distal termination link 312 on the distal end of the articulating link assembly. The proximal termination link may be held laterally by the composite insertion tube. The distal termination link is affixed to the camera assembly by the termination band. The articulation wires extending through the links may be laser welded to the termination band.

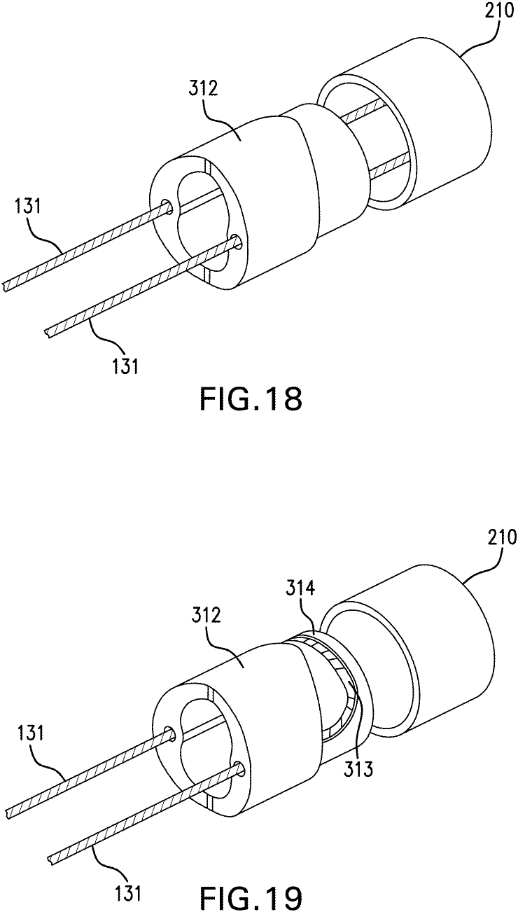

FIG. 18 illustrates the mounting of the distal end of the articulation wire at the distal end of the insertion tube. In one embodiment, the distal end of the articulation wire extends through the distal termination link and may be laser welded to the termination band. FIG. 19 illustrates a second embodiment for mounting the distal end of the articulation wire at the distal end of the insertion tube. The articulation wire may be laid within a groove 313, for example a U-shaped groove, in a stepdown portion of the distal termination link. The articulation wire is secured, for example by an adhesive and locked into place by sliding the termination band over the stepdown portion 314 of the distal termination link. In both embodiments, the termination band provides a secure connection between the distal termination link and the cap assembly. Advantageously, both embodiments allow for the fixation of the distal end of the articulation wire within the limited space of the small diameter endoscopes.

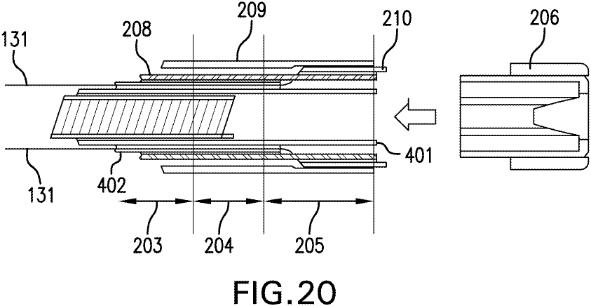

FIG. 20 illustrates an alternative embodiment for steering/articulating the articulation section of the insertion tube. This alternative embodiment may not utilize articulating links. As described above, the insertion tube includes the proximal section, the transition section and the articulating section. The insertion tube includes the exterior polymer jacket and the braid on the interior of the jacket. Interior to the braid, a polytetrafluoroethylene (PTFE) sleeve 402 extends from the proximal section to the articulating section. The articulating wires extend through the sleeve and are secured to the walls of the termination band behind the braid, for example by welding. The articulating wires may be flat pull wires. A main shaft PTFE liner 401 is situated on a side of the PTFE sleeve opposite the braid and defines the main lumen of the insertion tube. Advantageously, this arrangement eliminates the need for links and increases the available area in the distal end of the insertion tube. This increased available cross-sectional area facilitates the free movement of the tool channel, the electrical signal conductors and any other extrusions that may be located within the main lumen of the insertion tube. This arrangement may allow the distal end of the insertion tube to bend more than three hundred degrees and, in some arrangements, up to three hundred and ten degrees.

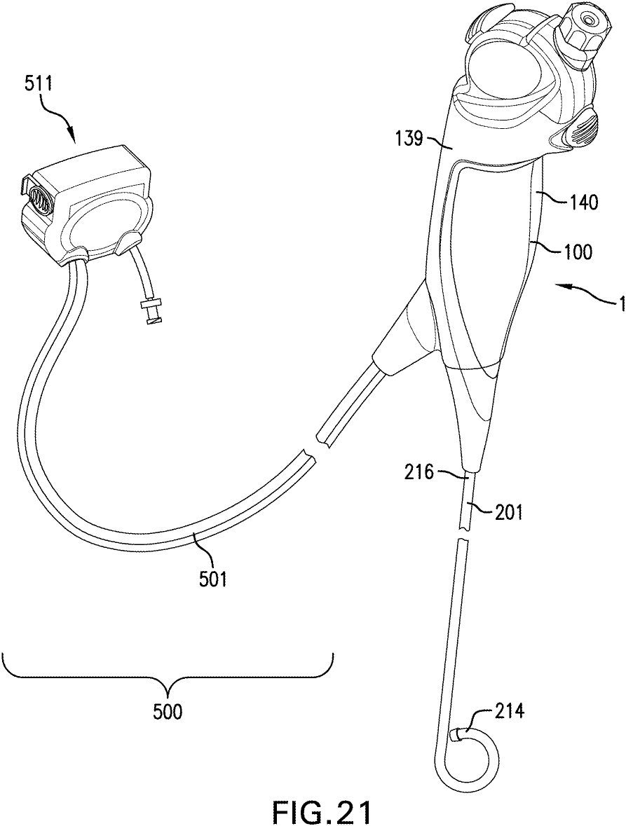

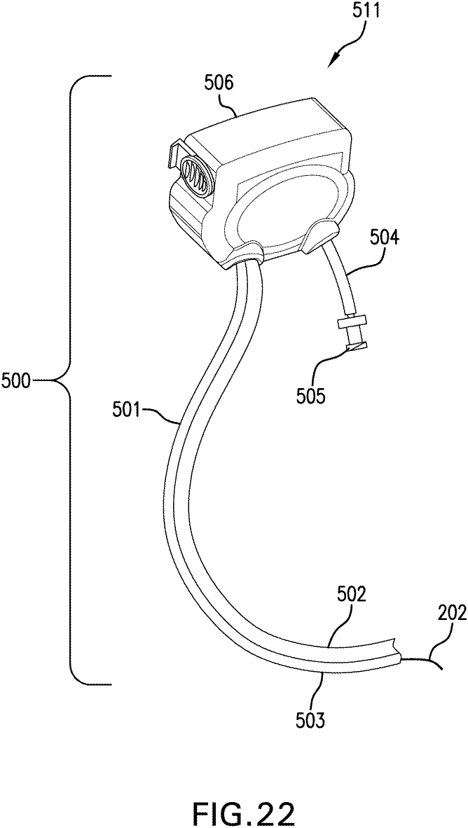

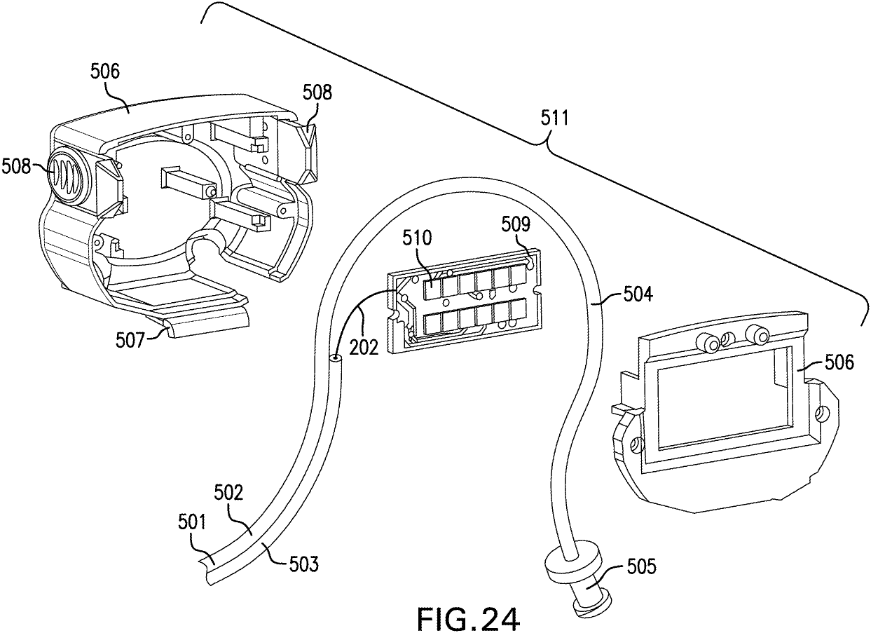

Turning to FIGS. 21-24, illustrated is an umbilical assembly 500. The umbilical assembly includes the umbilical 501 and a first cartridge 511. As described above and illustrated in FIG. 22, the umbilical includes fluid lumens and the electrical signal conductors extending between the cartridge and the endoscope’s hand assembly. The umbilical is attached on one end of the umbilical to a cartridge at one position on the cartridge 511. The cartridge 511 may include a housing 506. A second tube or a second portion of umbilical 504 may extend through the housing and extend out of the housing from another location on the housing. In some embodiments, the second tubing is connected to the first irrigation source described above. The second tubing may include a Luer lock connector 505 at one end of the tube to connect the second tubing to the first irrigation source. The second tubing may extend through the housing of the cartridge and into the umbilical (as demonstrated in exploded FIG. 24). Advantageously, in case of a manual or mobile irrigation source, the second tube will reduce the chance of movement of the first irrigation source pulling the endoscope handle during a procedure.

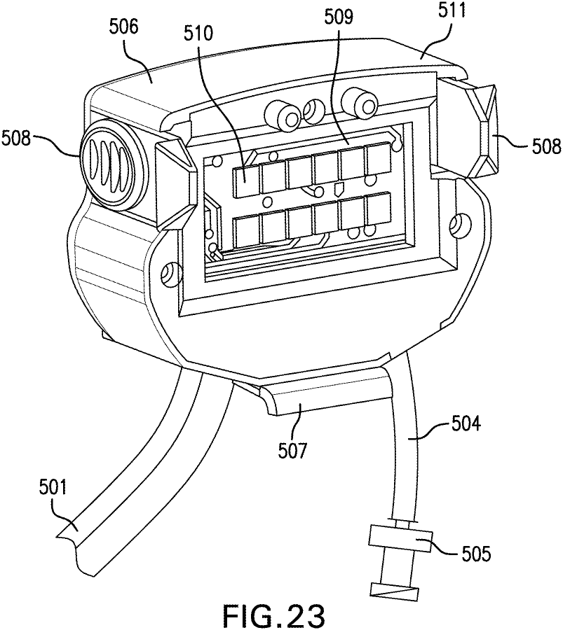

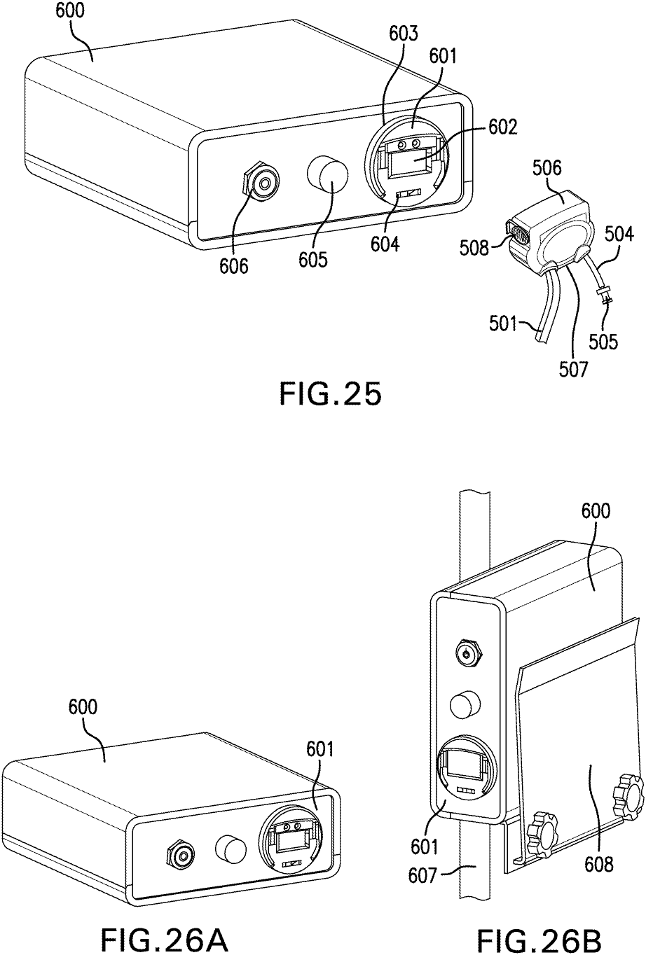

FIGS. 23 and 24 show additional detail of the assembled cartridge 511 and an exploded cartridge 511 respectively. The cartridge 511 may include a printed circuit board (PCB) 509. The PCB may include electrical signal spring tabs 510. The cartridge spring tabs may interface with the console 600 or 700 to establish an electrical connection between the console 600 or 700, the cartridge 511 and the endoscope. The cartridge 511 may include a combination of static retention tabs 507 and/or dynamic retention tabs 508. The retention tabs operably connect the cartridge to a receiving receptacle 601 (illustrated in FIG. 25) of the console by connecting to corresponding retention slots, including static retention slots 604 and dynamic retention slots 603. In some embodiments, some of the retention tabs may provide for a “snap on” connection of the cartridge to the console. The ability to snap the cartridge on the console allows for a quick and secure method for attaching the cartridge to the console or detaching the cartridge from the console.

Regarding FIGS. 25, 26A and 26B, a first console 600 without fluid control is illustrated in additional detail. Without fluid control, the console 600 may be relatively small so as to save space within the operating theater and on carts that may be in use. The console 600 may include a receptacle 601. The receptacle may include an electrical signal connector 602 to connect the electrical signal spring tabs of the first cartridge 511 to the console 600. The receptacle may further include retention slots to receive and connect the dynamic retention tab of the cartridge and a retention slot for the static retention tab of the cartridge 511. This connection creates an electrical connection between the console 600 and the cartridge 511. The dynamic retention tabs may be automatically released from the dynamic retention slots if an excessive amount of tension is placed on the umbilical. Advantageously, this safety release prevents unintended damage to the console or cartridge. In some embodiments, only the static retention tabs and slots are utilized. In other embodiments, only the dynamic retention tabs and slots are utilized. In additional embodiments, both the static and dynamic retention tabs and slots are utilized.

The console 600 may further include an illumination control 605 to control the power to the LED at the distal end of the insertion tube. The illumination control may also control the wavelength of the light emitted from the LED. The console may also include a power control 606 to power the console on or off. For example, the power control may be a switch, knob or button.

FIG. 26b illustrates the cartridge attached to a support pole 607. The cartridge may include a bracket 608 to attach the console to a pole in a vertical configuration. As seen in FIGS. 26a and 26b the receptacle may be oriented to allow the cartridge to be connected to the cartridge in the same configuration whether the console is horizontal or vertical. In other words, the receptacle may be rotated to a desired orientation based on the orientation of the console. Advantageously, this allows the desired ergonomics and tubing arrangement to be maintained regardless of console orientation within the operating theater.

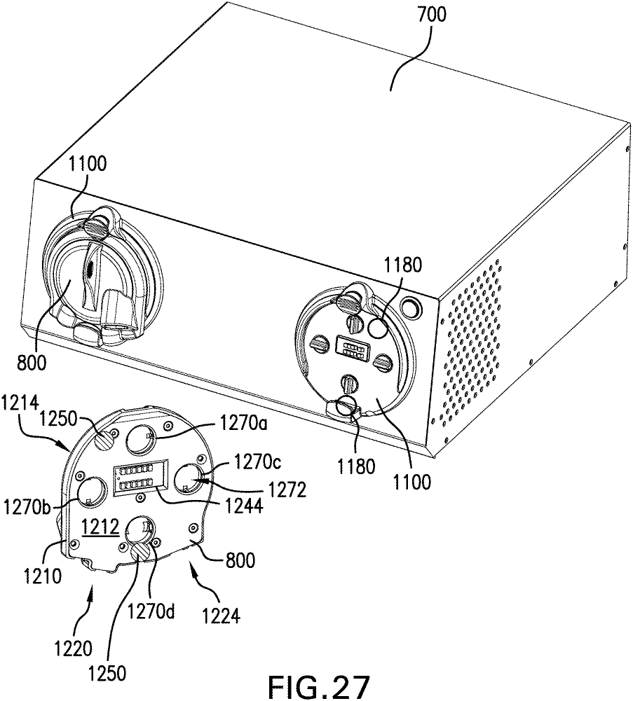

The present disclosure further provides a second cartridge 800 and a second console 700. FIG. 27 generally illustrates the system operationally connected for use, preferably by a medical professional. The second cartridge 800 and second console 700 may be larger than the first console 600 and first cartridge 511 due to the use of fluid control mechanisms with the second console 700 and second cartridge 800. The system may include the cartridge 800 inserted into a control valve assembly 1100 of the console 700. The cartridge 800 can be connected to the endoscope by the umbilical. For instance, the cartridge 800 may be connected to a single-use endoscope by one or more fluid tubes and/or electrical conductors.

The cartridge 800 may be connected to support equipment. The support equipment may include an irrigation pump, an insufflator, and/or a vacuum pump (e.g., stand-alone or central). The cartridge 800 may be positioned between the endoscope and the support equipment.

In some examples, the console 700 may include a user interface (not shown) allowing the user to alter the various variables of the endoscope system. In one example, the user interface may be a touchscreen integrated into an electric visual display. In another example, the user interface could include a keyboard, mouse, trackball, and/or touch sensitive pointing device, etc.

The console 700 may include a control valve assembly for receiving and actuating upon a cartridge. The console 700 may provide electrical power, fluids and/or fluid pressure, vacuum pressure, and/or send and/or receive electrical signals with the endoscope assembly. The console 700 may include a monitor (not shown) for visualizing signals received from the endoscope assembly such as a video feed from a camera at the distal end of the single-use shaft assembly.

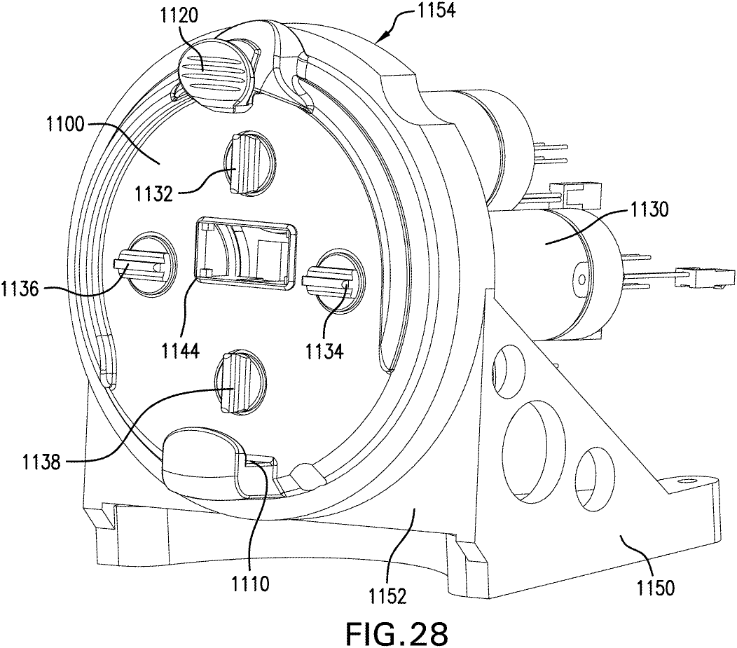

With reference to FIG. 28, the control valve assembly is shown in greater detail. The control valve assembly may be used to selectively actuate an actuatable portion of fluid paths of the cartridge 800. The control valve assembly may include actuators 1130 and a support structure 1150 comprising a first surface 1152 and a second surface 1154.

The actuator may be a linear actuator or rotational actuator. The actuator may be a solenoid. Although as few as one actuator is appreciated by the applicant, in preferred examples the control valve assembly includes four actuators. The actuators are arranged to actuate valve portions of fluid paths in the cartridge to selectively open and/or close the fluid paths.

The valve portions of the fluid paths may comprise any acceptable valve. For example, the valve portion may comprise a petcock. The valve portion may also comprise a portion of tubing arranged for compression (e.g., a pinch valve) between the anvil on the first surface of the door and the actuator (e.g., solenoid) of the control valve assembly. The valve portions may serve as a valve for any number of fluid functions of the endoscope, such as a suction valve associated with actuator 1132, a camera flush valve associated with actuator 1134, an insufflation valve associated with actuator 1136, and/or an insufflation vent valve associated with actuator 1138.

The actuators of the control valve assembly include electrical connectors to be received by the console 700 and electrically connect the actuators to the console 700. When connected to the console 700, the console 700 can provide electrical power and user inputted commands to the actuator to open and/or close the fluid pathways of the cartridge.

The cartridge 800 may include an identifier readable and/or writable by the control valve assembly. The identifier may include information regarding the type of cartridge and/or usage information (e.g., whether the cartridge has been used before). Preferably, the control valve assembly is configured to read and process the identifier of the cartridge 800 (e.g., to determine the type of cartridge and/or whether the cartridge has been used previously). The identifier may be readable and/or writable through an/the electrical connector of the cartridge and/or through other means (e.g., RFID, optics, and/or mechanical contact).

The first surface of the support structure may include the electrical connector for forming electrical connections to the electrical connector of the cartridge 800. The electrical connection between the control valve assembly of the console 700 and the cartridge 800 may allow the console to receive images and control data (valve/actuator and/or image data) passed from the endoscope through the cartridge 800 to the console 700. The electrical connection may allow the console 700 to supply power to the light-emitting diode (LED) of the endoscope assembly.

The cartridge can be a single-use cartridge. The single-use cartridge can negate the need for the use of permanent valve bodies contained within a reusable endoscope and/or the console. This advantageously eliminates the need to sterilize reusable valves after every use.

The cartridge 800 has a housing 1210. The housing can include a first side 1212 and a second side 1214. The housing further comprises a first fluid side 1220 and a second fluid side 1224 allowing for fluid to travel in and out of the cartridge housing. The housing may define one or more fluid pathways, fluid connector, and/or electrical connectors. For example, cartridge housing can include electrical connector 1244 in electrical communication with an electrical conductor of the endoscope and optionally an optical sensor module that contains optical sensor calibration data or a unique identifier for the endoscope. The housing may define windows 1270a-d. In one example, the housing includes four windows. The windows may be defined by inner surfaces of the housing. The inner surface may create a generally circular opening in the housing. The circumference of one window may be larger than the other windows to receive a larger fluid path cross section.

The windows can be configured to receive an actuator from the console into a chamber 1272 defined by the inner surface. As described above, the actuator may pass through the window and/or contact an anvil on the door of the control valve assembly of the console 700 to create a pinch valve with the door. When the cartridge 800 is positioned on the control valve assembly and the door is closed, the windows align with the anvils on the door and the actuators of the control valve assembly. The windows may be arranged to define a geometric shape. For instance, as illustrated, the windows may define a diamond.

As described above, one or more actuators may be solenoids configured to extend into the windows of the cartridge housing and/or contact the anvils on the door of the control valve assembly creating a pinch valve when closed.

The housing may further include an electrical connector for connecting to the electrical connector of the control valve assembly. The electrical connector of the cartridge 800 may be spring finger electrical contacts configured to contact a corresponding number of flat pad electrical contacts on the control valve assembly of the console or vice versa. The electrical connector of the cartridge housing may pass images and control data provided by the endoscope to the console. The electrical connector of the housing may pass power from the console to the LED used by the endoscope in the patient’s body.

Looking to FIG. 27, a view of the cartridge 800 inserted into the control valve assembly of the console 700 is shown. As can be seen, the cartridge is inserted flush with the first surface of the support structure of the control valve assembly. The first fluid side of the cartridge may include loose tubing that continues into the umbilical and to the single-use shaft assembly of the endoscope (not shown).

The second fluid side of the cartridge 800 may be connected to support equipment. The support equipment may include an irrigation pump, an insufflator, and/or a vacuum pump (stand-alone or central). The cartridge 800 may provide for fluid communication between the endoscope and the support equipment.

The control valve assembly may include a ledge surface 1110 and a latch 1120. The cartridge 800 may include a ledge surface and a latch receiving portion. In alternate examples, the control valve assembly may include the latch receiving portion and the cartridge may include the latch. When the cartridge is received by the control valve assembly the latch contacts the latch receiving portion to secure a first end of the cartridge to the control valve assembly. Separately, and opposite of the latch and latch receiving portion, the ledge surface of the cartridge and the ledge surface of the control valve assembly contact each other to secure a second end of the cartridge to the control valve assembly.

In some examples, the cartridge 800 may include two or more magnets 1250 located on the first side of the cartridge. In some examples, one magnet may be located at the bottom of the first side of the cartridge 800 and the second magnet located at the top of the first side of the cartridge. The first surface of the control valve assembly may include two or more magnetic sensors 1180 (hall or reed), mirroring the locating of the magnets on the cartridge. For example, one located at the bottom of the first surface and the other located at the top of the first surface. The bottom magnet on the cartridge 800 interfaces with the bottom magnetic sensor on the control valve assembly triggering the actuator (e.g., solenoid) valves to retract reducing the force required to tilt the cartridge into the control valve assembly. The top magnet interfaces with the top magnetic sensor on the control valve assembly to confirm the presence of the cartridge 800 on the console 700, triggering the console 700 to release the appropriate actuators and initiate the actuator operation mode for endoscopy.

FIG. 27 illustrates the console 700 with one cartridge 800, an additional cartridge 800. One cartridge 800 may be connected to a first endoscope (not shown) and the additional cartridge 800 may be connected to a second endoscope (not shown). One cartridge 800 and the additional cartridge 800 may be securely inserted into a first control valve assembly and a second control valve assembly of the console, respectively. Although the console is illustrated with the first and second control valve assembly, the console may have any number control valve assemblies each capable of receiving an independent cartridge. The first control valve assembly may receive the first cartridge 800 and the second control valve assembly may receive the second cartridge 800. It is not necessary for every control valve assembly to receive a cartridge during every medical procedure.

As illustrated, each cartridge may be connected to a separate set of support equipment and an independent medical device, such as an endoscope.

The first control valve assembly and the second control valve assembly are configured to operate independently and/or in a main/secondary communication configuration. The first or mother endoscope may be the main controller and the second or daughter endoscope may be the secondary controller. In this arrangement, the controls of the main endoscope may control the fluid functions (e.g., image controls and/or LED) of one or more secondary endoscopes. The main endoscopes may control the function of the fluid paths of the secondary endoscopes by providing an electrical signal to the second control valve assembly to selectively actuate an actuator as described herein (e.g., through one or more cartridges connected to the console). The first endoscope may further control a function of the second endoscope (e.g., operation of the LED and/or camera) by providing an electrical signal from the first endoscope to alter a condition of the second control valve assembly (e.g., alter an electrical signal provided by the second control valve assembly to the cartridge of the second endoscope). For instance, the control valve assembly may provide an electrical signal to the LED and/or the camera of the second endoscope based on the signal received from the first endoscope. This arrangement further allows for multiple endoscopes to be setup and utilized in a procedure at once, eliminating or reducing extensive setup time typically used to change one endoscope out for another endoscope and/or eliminating or reducing the need for additional equipment, such as multiple control consoles.

The console is illustrated with a touchscreen user interface. The user interface may be any suitable input/output device.

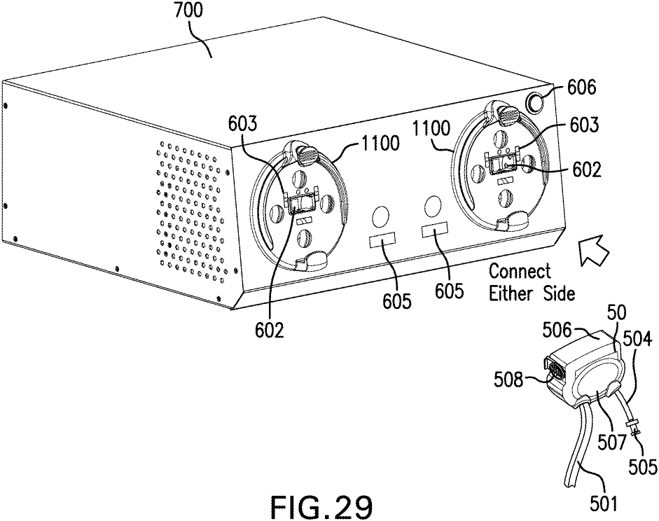

Turning to FIG. 29, the second console 700 is shown with the first cartridge 511. The second console 700 may include fluid controls and may be larger than the console 600 without fluid controls. The second console 700 may include one or more receptacles/control valve assemblies. The receptacles may include an electrical signal connector to connect the electrical signal spring tabs of the cartridge 511 or 800 to the console. The second console 700 may allow a user to attach a cartridge with fluid control or without fluid control. In some examples, a large cartridge 800 (illustrated in FIGS. 27 and 28) may be connected to the receptacle of the large second console 700. The larger second cartridge 800 may cover a portion of the receptacle. In this example, as described in detail above, the console 700 includes valves that may actuate to control fluid flow through the cartridge 800. In another example, the first cartridge 511 described above may be secured to cover a smaller portion of the receptacle of the second console 700 than the large cartridge 800. In a third example, a large cartridge 800 may be used to cover the entire receptacle, but the fluid controls are not utilized. As described above and illustrated in FIG. 29, the console 700 can include multiple receptacles/control valve assemblies. Any variety of the cartridges 511 and 800 may be secured to the console 700 and used at one time.

Advantageously, for example, attaching the cartridge 511 without fluid control to the larger console 700 provides the ability of a clinician to connect a cytoscope to either a pole mounted console located in a medical office setting or to the dual console 700 (illustrated) in an operating room setting for ureteroscopy. Further, a single-use cholangioscope may be used in conjunction with a hybrid duodenoscope during endoscopic retrograde cholangiopancreatography such as by connecting both to a dual console 700. Additionally, a single-use cholangioscope could be used with the smaller console 600, without fluid control, in conjunction with a third party reusable duodenoscope and its associated video processing unit.

It is understood that many features of the various cartridge embodiments and the various console embodiments may be used interchangeably between the embodiments. In other words, the features of the various cartridges and consoles may be used within any of the embodiments.

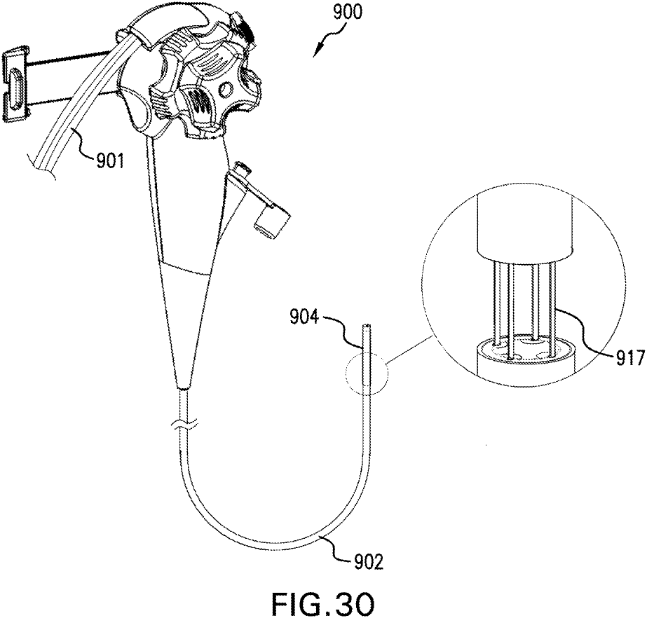

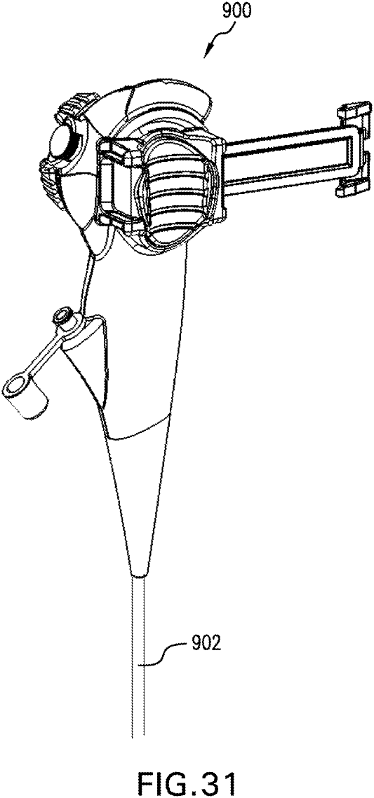

Turning to FIGS. 30-36, an improved 4-way steering flexible endoscope is disclosed. The endoscope is illustrated as a small diameter endoscope, and more specifically a cholangioscope. However, it is understood that the 4-way steering can be incorporated into other small diameter endoscope, large diameter endoscopes and other medical devices in general.

FIGS. 30 and 31 illustrate an endoscope assembly 900 with an umbilical cord 901 and insertion tube 902. All discussion of the umbilical and insertion tube above is incorporated herein. The insertion tube 902 includes an articulating portion 904. The insertion tube further includes articulation wires 917. The articulation wires are used to articulate the articulating portion of the insertion tube. In one embodiment, the insertion tube includes two articulation wires for either up-down or left-right articulation. In one exemplary embodiment, the insertion tube includes four articulation wires. This configuration allows for up-down and left-right articulation by the user.

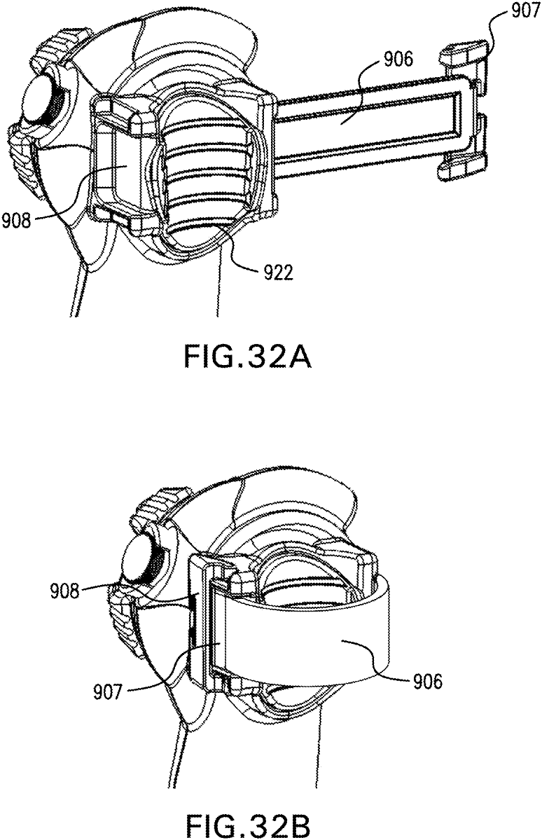

FIGS. 32A and 32B illustrate a mounting structure to mount one endoscope onto the handle of another endoscope. The ability to mount and secure one endoscope to the other allows the endoscopist to handle both endoscopes easier and decreases the cumbersome nature of the two endoscopes. Often, a small diameter endoscope is deployed through the tool channel of a larger diameter endoscope, for example a cholangioscope through a duodenoscope. The mounting structure is arranged to secure the small diameter endoscope to the handle and/or strain relief of the large diameter endoscope.

The mounting structure includes an elastic strap 906 with an alignment pad 922, for example an elastomeric pad. In some embodiments, the alignment pad is curved to match the typical shape of the body of a large diameter endoscope. The alignment pad prevents movement of the small diameter scope on the large diameter scope. The mounting mechanism further includes a cam latch 907 and land for the cam latch 908. FIG. 32a illustrates the mounting structure in the open position with the cam latch free of the land for the cam latch. FIG. 32b illustrates the mounting structure in a close/affixed position with the cam latch secure to the land for the cam latch. In the closed position, the small diameter endoscope may be securely affixed to the body of a large diameter endoscope.

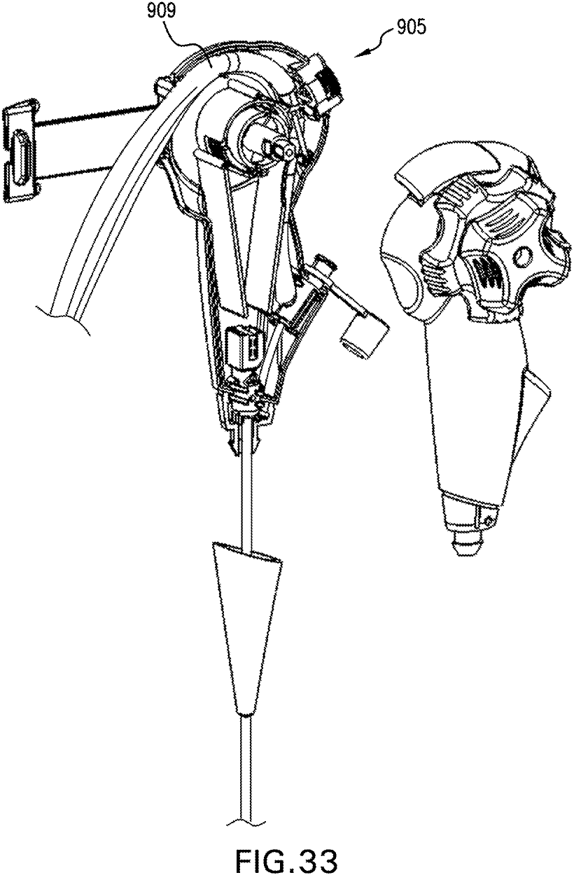

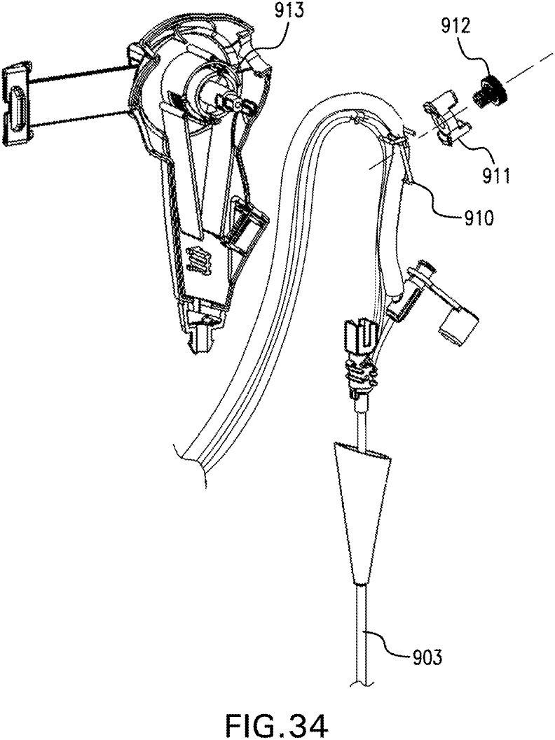

FIGS. 33 and 34 illustrate a variable integral suction control integrated into the handle of the endoscope. The suction control allows for variable manual suction actuation. Further, it allows for selection of a variable suction strengths. The valve could additionally control the flow of irrigation fluid.

The suction control includes an integral variable suction valve 905 and a suction tube 909. The integral suction valve includes a valve variable screw 912, a valve sliding button 911, a valve spring 910 and a valve anvil 913. The screw may be rotatable or compressed without rotation. As the screw is rotated, the suction tube is either compressed against the valve anvil to lower the strength of suction or the compression against the valve anvil is released to increase suction. The level of suction may range from no suction, if the suction tube is completely shut off against the valve anvil, to completely open if the screw is not compressing the suction tube against the anvil. By adjusting the screw, a user is able to obtain a variable amount of suction.

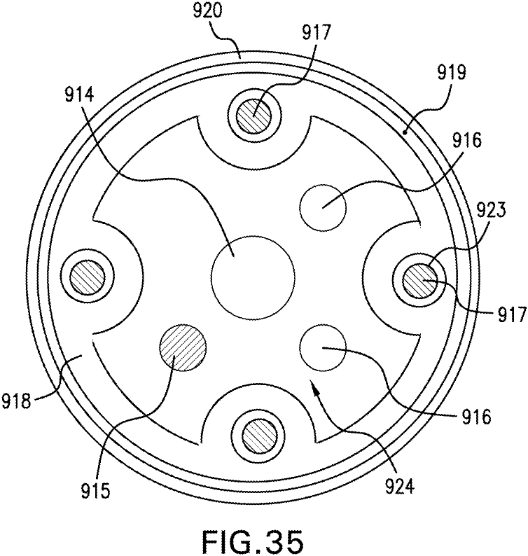

FIG. 35 illustrates a cross sectional view of insertion tube 903. The insertion tube may be a solid Polytetrafluoroethylene (PTFE) extrusion with integral lumens extending within the insertion tube (as described below). As illustrated, the insertion tube may include a lumen defining a tool channel 914 for extending various medical devices through the insertion tube and into the body of a patient. The insertion tube may further include another lumen for one or more electrical conductors 915 to pass an electrical signal to a camera and/or light diode at the distal end of the insertion tube, for example for a complementary metal oxide semiconductor (CMOS) sensor wire. The insertion tube further includes fluid tubes 916, such as an irrigation tube or a suction tube 909. As discussed above, the insertion tube further includes articulation wires 917 to articulate the insertion tube. Finally, the insertion tube includes a core profile 918 surrounded by a braid 919.

The core profile may be fabricated of a lubricious polymer incorporating a central lumen 924 with four articulation wire lumens 923 to extend the articulation wires through. In some embodiments, the central lumen is PTFE. In one exemplary embodiment, the articulation wire lumens 923 are equally spaced around the circumference of the central lumen. The four lumens (lumen for the tool channel, the lumen for the electrical conductors and the two fluid lumens) may be made of nylon, EVA, and/or PVC.

The braid 919 surrounds at least a portion of the exterior of the core profile’s central lumen. The braid is encompassed by a sheath/outer jacket 920. The outer jacket may comprise HDPE, EVA, PVC, and/or PBAX.

During manufacture of the insertion tube, the lumen defining the tool channel, the lumen with the electrical conductors extending through them, various fluid tubes and articulation wire lumens are pulled through the central profile’s central lumen. In one embodiment, the lumens are all pulled at once. This limits manufacturing method limits the number of leak points.

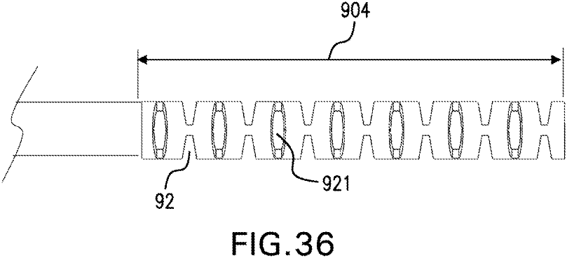

Turning to FIG. 36, a side view of the articulating section of the insertion tube is illustrated. The articulating section may include two opposed series of articulation cuts 921 in the core profile. These cuts are made throughout at least a portion of the length of the articulating section. These cuts create a series of articulating living hinges that promote bending in a single plane, for example at ninety degrees to one another. The articulating section may be fabricated from a different material from the core profile, for example nylon. The articulating section may be separably attached to the rest of the insertion tube via an adhesive, by a mechanical connection, or by virtue of the articulation wires extending through the articulating section and the rest of the insertion tube.

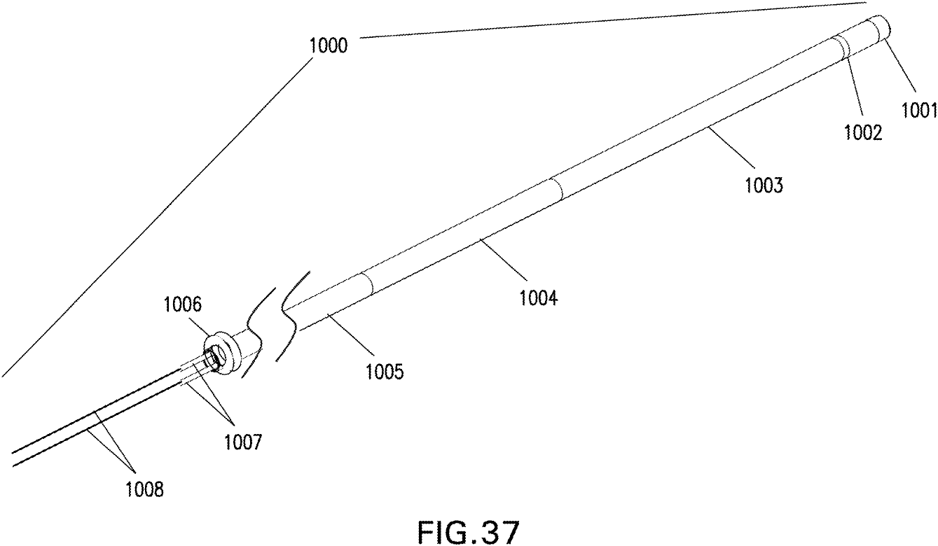

Turning to FIGS. 37-48, another insertion tube 1000 is disclosed. The disclosure of the insertion tubes disclosed above are incorporated herein. The differences will be discussed in relation to insertion tube 1000.

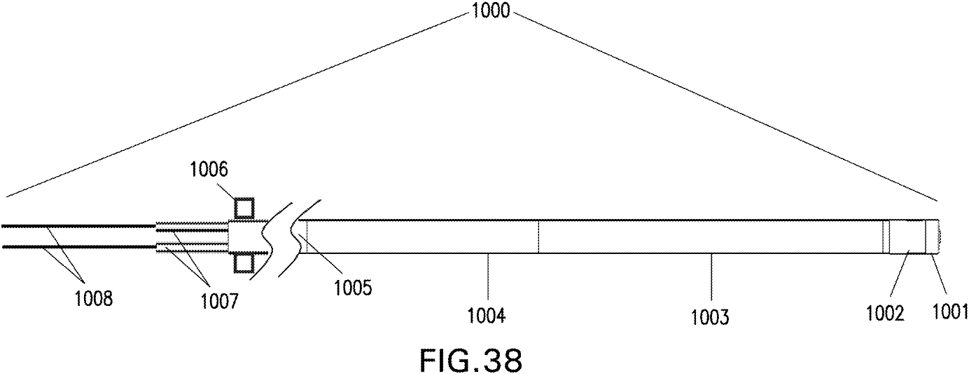

Regarding FIGS. 37 and 38, the insertion tube may have multiple sections including a proximal section 1005, a transition section 1004 and an articulating section 1003 located at the distal end of the insertion tube. The proximal section of the insertion tube may be compromised of higher durometer polymers than the articulating section to prevent bending of the proximal section during articulation of the articulating section. The articulating section may be made of lower durometer polymers than the proximal section to allow the articulating section be bent by the articulating wires. The transition section is operably located between the articulating section and the proximal section. The transition section may be made of polymers with a higher durometer measurement than the articulating section and a lower durometer measurement than the proximal section. This allows the transition section to passively bend during articulation of the articulation section of the insertion tube. Therefore, when the articulating section is articulated there is a smooth, fluid transition between the unmoving or minimally moving proximal section and the articulating section.

The insertion tube may further include a camera cap assembly 1001 located at the distal end of the insertion tube. The insertion tube further includes a transition band 1002 between the articulating shaft section and the transition band. The diameter of the transition band is larger than the diameter of the cap assembly so that the cap assembly may be inserted into an opening of the termination band and secured thereto.

FIGS. 37 and 38 further illustrate an insertion tube termination fitting 1006. The insertion tube termination fitting may receive articulation compression coils 1007 that receive one or more articulation coils wrapping around respective articulation wires 1008. The articulation wires are configured to bend a distal shaft section of the endoscope assembly upon actuation. The insertion tube termination fitting further assists in securing the insertion tube to the endoscope handle body.

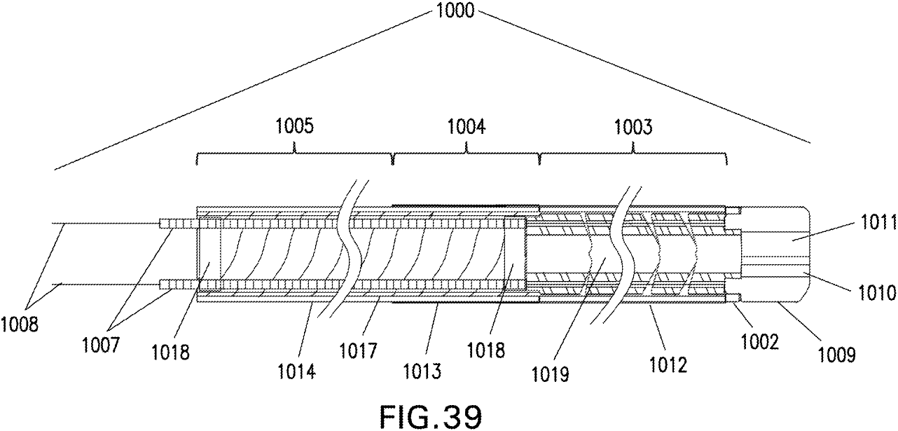

FIG. 39 illustrates a cross-sectional side view of the insertion tube. The camera cap assembly 1001 may include a camera cap 1009, an optical sensor (camera) 1010 within the camera cap and a tool channel 1011. The tool channel extends from the endoscope handle body through the insertion tube and exits out of the camera cap.

The insertion tube includes an articulating section polymer jacket 1012, a transition section polymer jacket 1013 and a proximal section polymer jacket 1014 encompassing the articulation section, transition section and proximal section, respectively. The three jackets compose an outer composite tube 1046. The outer composite tube may be assembled in a single operation on a mandrel prior to insertion of any of the interior components of the insertion tube.

The insertion tube includes a plurality of articulating links 1019 extending throughout the insertion tube. The plurality of links are formed of a series of two or more links. The links facilitate articulating the distal portion of the insertion tube (articulating shaft section) within a patient’s body. The use of distal articulation links creates a greater degree of articulation based on a given input force when tools are in place within the tool channel as compared to other methods.

FIG. 39 further illustrates coil 1017 and heat stake 1018 attaching the coil to the proximal shaft section. The insertion and heat staking of the coil in the proximal shaft section, secondary to the initial shaft layup simplifies fabrication.

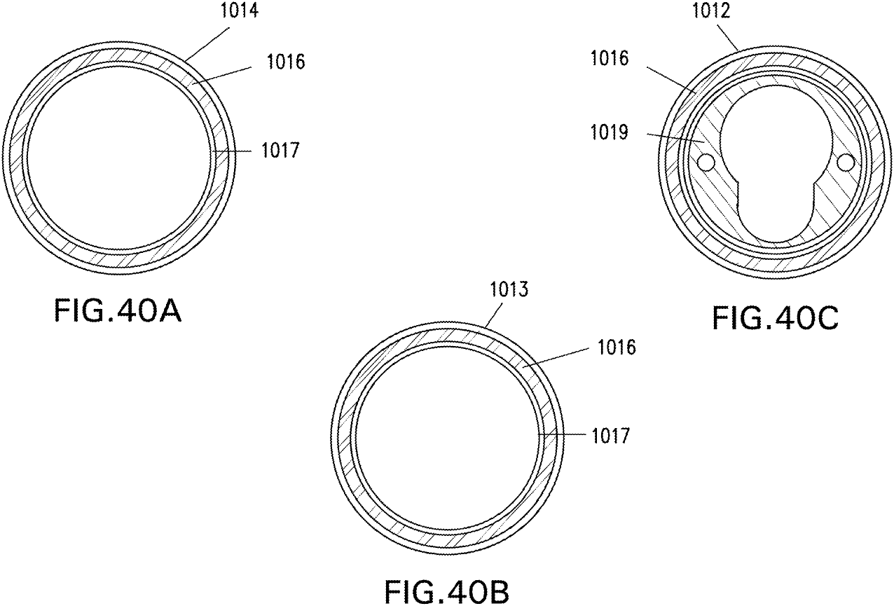

FIG. 40A illustrates a cross sectional view of the proximal shaft section. The proximal shaft includes the outer proximal section polymer jacket. The polymer jacket may extend circumferentially around the proximal shaft section. A braided wire 1016 extends within the polymer jacket 1014. The coil 1017 is shown within the braided wire 1016.

FIG. 40B illustrates a cross sectional view of the transition shaft section. The transitional shaft section may be identical to the proximal shaft section with the transition section polymer jacket instead of the proximal section polymer jacket.

FIG. 40C illustrates a cross sectional view of the articulating shaft section. The articulating shaft includes the outer articulating section polymer jacket. The polymer jacket may extend circumferentially around the articulating shaft section. A braided wire 1016 extends within the polymer jacket. The braided wire encircles the assembly of articulating links 1019.

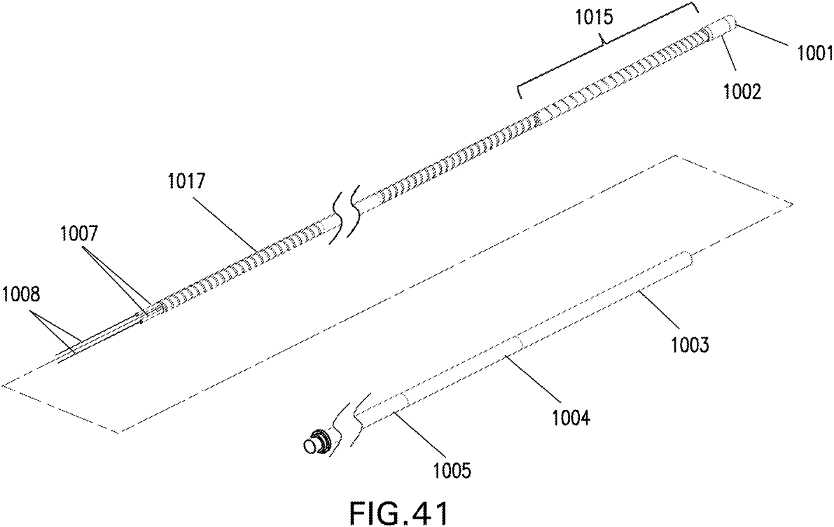

FIG. 41 illustrates a partially exploded view of the insertion tube. As illustrated, during manufacturing of the insertion tube, the articulation components (coils 1017, articulation wires 1008, links 1019 and internal tubing) may be pulled as a singular unit into the outer composite tube 1046. As illustrated, during manufacturing, the proximal shaft section including the compression coils and articulation wires are extended through the articulating shaft section and pulled through the composite insertion tube until the proximal shaft section meets the insertion tube termination fitting and the compression coils and pulls wires extend past the insertion tube termination fitting. Pulling all the interior articulation components as a whole can make assembly it easier. Advantageously, articulation coils 1007 can prevent unintended movements of the proximal shaft section by isolating articulation forces from the proximal shaft.

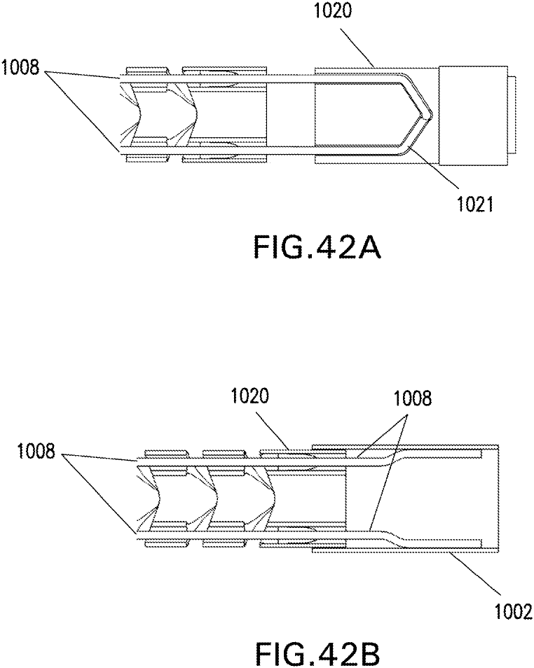

FIG. 42A illustrates the mounting of the distal end of the articulation wire at the distal end of the insertion tube. The articulation wire may be positioned within a groove 1021, for example a U-shaped groove, in a stepdown portion of a distal articulation link 1020. The articulation wire is secured, for example by an adhesive and locked into place by positioning of the transition band over the distal articulation link. This provides a simple method of fixation with a continuous wire, thus limiting the risk of a loose wire end.

FIG. 42B illustrates another approach of mounting the distal end of the articulation wire at the distal end of the insertion tube. The distal end of the articulation wire extends through the distal articulation link and is affixed (e.g., laser welded) to the transition band.

In both embodiments, the transition band provides a secure connection between the distal articulation link and the cap assembly. Advantageously, both embodiments allow for the fixation of the distal end of the articulation wire within the limited space of the small diameter endoscopes.

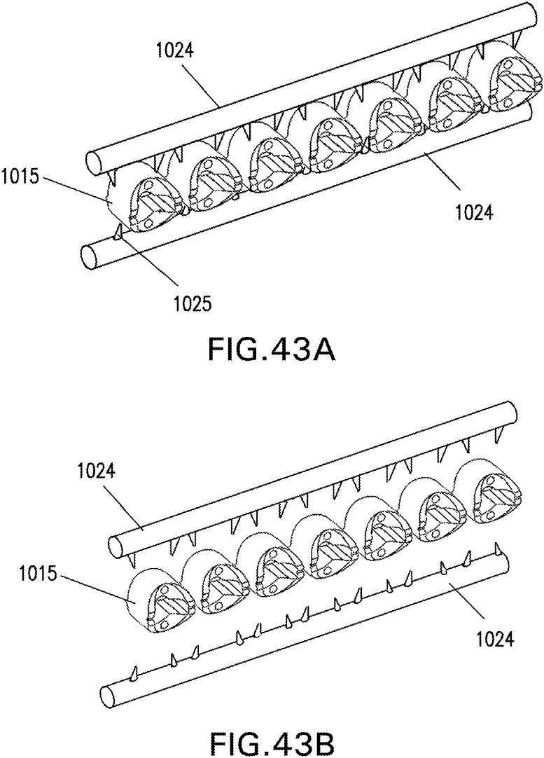

FIGS. 43A and 43B illustrate the plurality of links or printed links 1019 with support beams 1024 extending at least partially down a length of the plurality of links. The support beams include breakaway support struts 1025 attached to the plurality of links. The plurality of links may be formed during manufacturing of the plurality of links. As explained above, links of the plurality of links may different from one another. Such manufacturing may be injection molding, additive manufacturing, and/or subtractive manufacturing. The support beams and breakaway support struts may support the links and keep the links aligned during manufacturing. During assembly of the articulating link assembly, the support beams and support struts are removed and discarded.

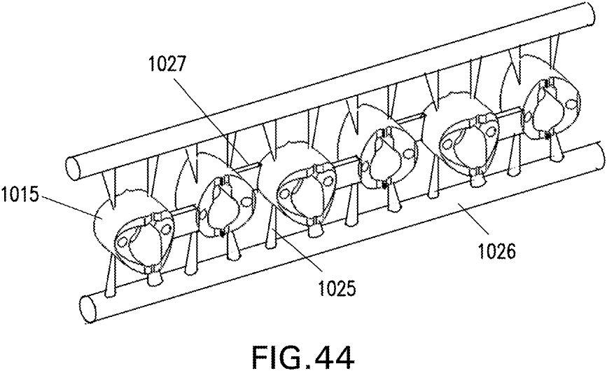

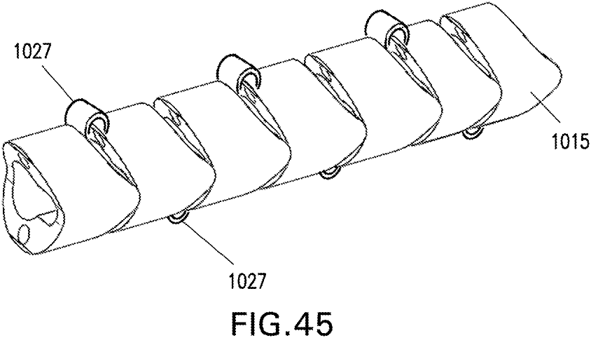

FIGS. 44 and 45 illustrate the plurality of links with a runner 1026 with breakaway support struts 1025 attached to the plurality of links. The runner may, support struts, and the links may be formed by injection molding. The links may be connected by flexible link connectors 1027. The flexible links connectors may be foldable after removal of the breakaway support struts and/or runner to pivot adjacent links of the articulating links towards one another. Advantageously, the flexible link connectors may aid in the proper ordering and orientation of the links during assembly.

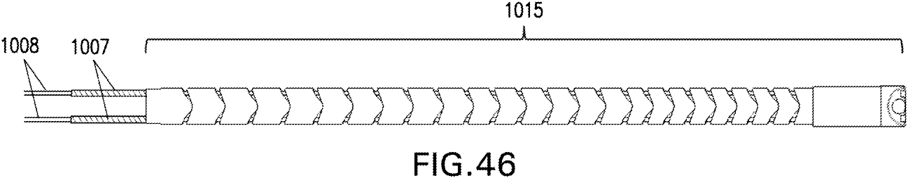

FIG. 46 illustrates a side view of the plurality of links with coils 1070 and wires 1080 in place.

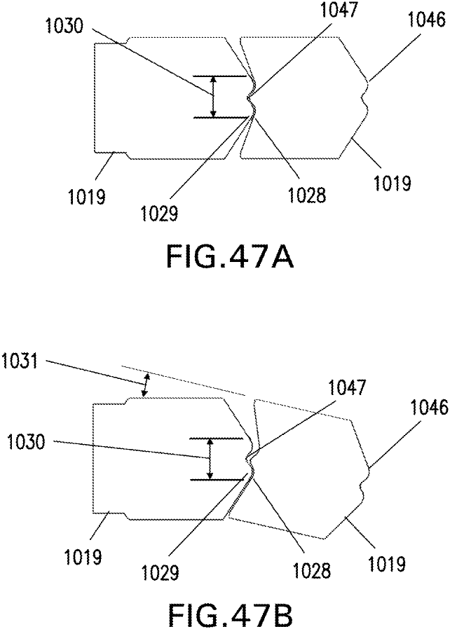

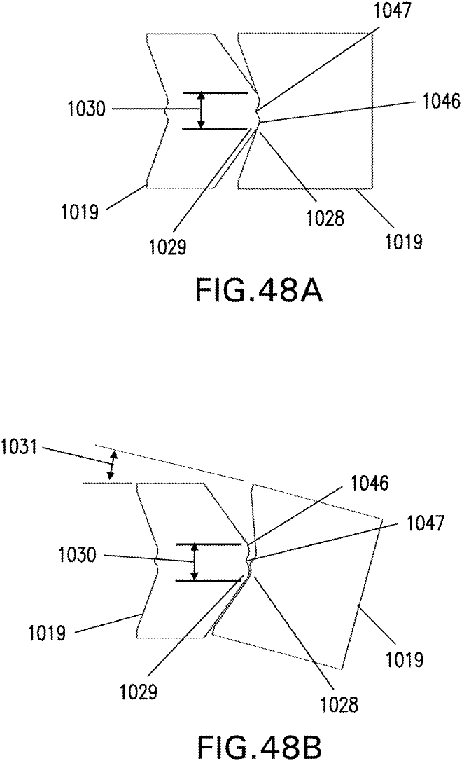

FIGS. 47 and 48 illustrate two assembled articulating links 1019. The disclosure of FIGS. 15 and 16 is incorporated herein. The proximal (bottom) end of the link includes two spaced apart concave/recessed articulating pivots 1028 and the distal (top) end of the link includes two corresponding convex/protruding articulating pivots 1029. Each concave/recessed articulating pivot 1028 and its corresponding convex/protruding articulating pivot 1029 form a pair.

When articulated to one side, such as that shown in FIGS. 47B and 48B, only one pair of the convex/concave articulating pivots may be in contact with one another. When articulated to a different side, a different pair of the convex/concave articulating pivots may be in contact with one another. Accordingly, when pivoted to one side, the adjacent links may articulate around a different pivot point than when the assembly is pivoted to a different side. In such instances, an articulation wire may be located a first distance from the pivot point when the assembly is articulated to a first side and a second distance when the assembly is articulated to a second side. Advantageously, such arrangements can allow an articulation wire to generate a greater moment (i.e., rotational force) for a given tensile force in the wire to pivot the adjacent links relative to one another when the assembly is articulated to one side verses another.

The plurality of links are formed of a series of two or more links. In some instances, the links are designed so that the distance 1030 between the pairs of convex/concave articulating pivots varies to control where articulation occurs first. By shortening the distance 1030 between the pivots about the centerline of the link, a given force may generate more articulation torque, as explained above. In a preferred embodiment, the distance 1030 between the convex/concave pivots decreases for successive pairs of convex/concave pivots along a direction toward the distal tip of the insertion tube. While articulating, an articulation angle 1031 is created between the links.

All of these features can be modified on individual links or in combination with multiple links to achieve the desired articulation response and geometry. Advantageously, this arrangement provides the ability to initiate articulation distally prior to or simultaneously with proximal articulation. Further, this arrangement provides the ability to mitigate increased pull forces that accumulate at the proximal end of the articulation section. Finally, this arrangement can provide a uniform curvature of the radius along the articulation section from the proximal end to the distal end.

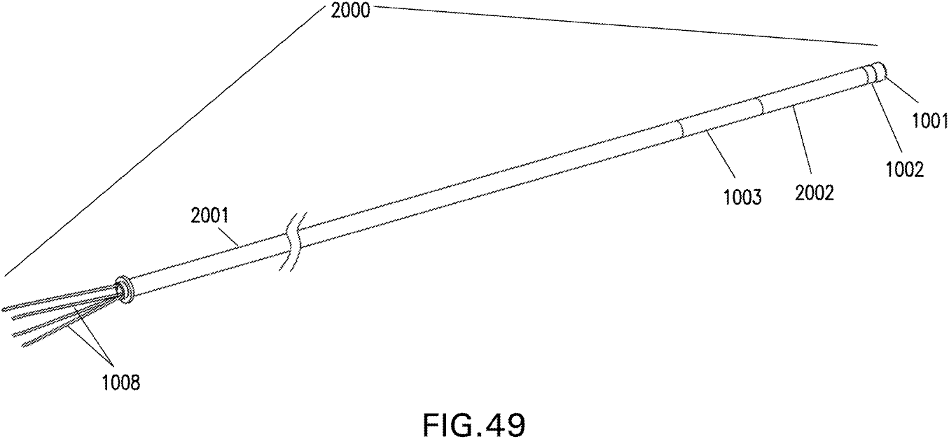

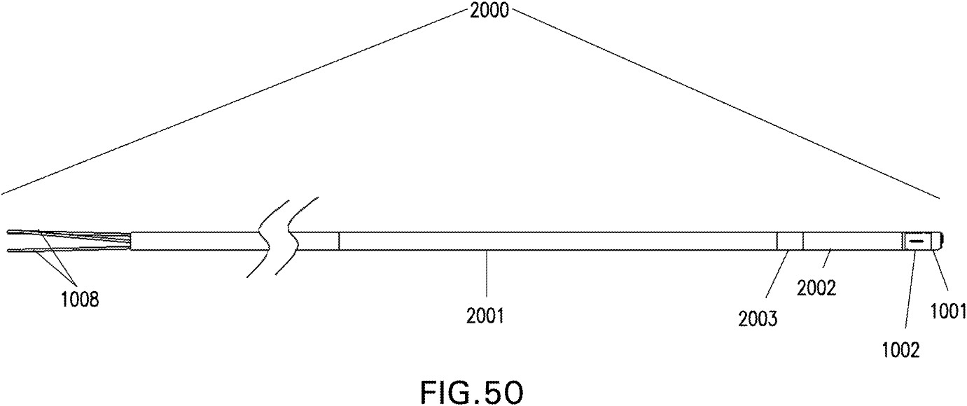

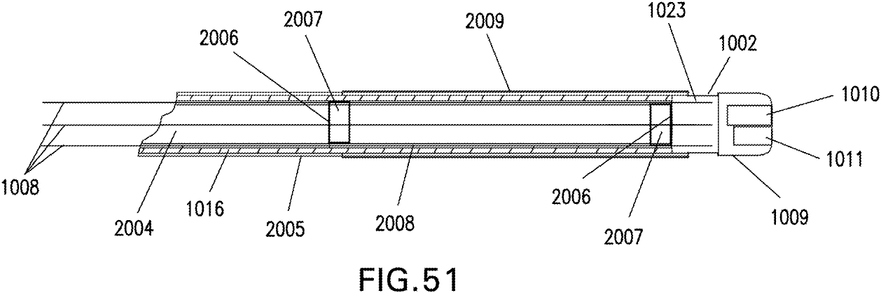

Turning to FIGS. 49-54, another insertion tube 2000 is disclosed. The disclosure of insertion tube 1000 is incorporated herein. The differences will be discussed in relation to insertion tube 2000. The insertion tube is composed of two sequential sections (proximal section and distal articulating section). The insertion tube includes a proximal multi-lumen shaft section 2001 with a multi lumen proximal extrusion 2004 and a deflection section 2002 illustrated as a steerable sheath. Between the two sections is a deflection transition section 2003. The two sections 2001 and 2002 are joined linearly by an exterior braid and multi-segmented polymer jacket.

The use of a proximal multi-lumen (including a central lumen and four articulation wire lumens as described above) shaft section 2001 simplifies the construction of the proximal shaft. The use of the central lumen facilitates pulling of the interior components, including the fluid lumens, lumen defining the tool channel, articulation wire lumens and electrical conductors as a single operation during assembly (as described above). The distal deflection section can be preassembled onto a mandrel along with the proximal multi-lumen extrusion prior to over-braid and reflow.

The insertion tube includes an outer proximal section polymer jacket 2005, an inner deflection transition section liner 2006, an outer deflection transition section jacket 2007, an inner deflection section liner 2008 and an outer deflection section jacket 2009.

Similar to insertion tube 1000 as described above, one or more pairs of articulation wires are bonded to the distal transition band. The transition band serves as a fixation point for the articulation wires and a transition to connect the distal tip of the deflection section with the camera cap assembly. The transition band provides a low-profile connection between the deflection section and the camera assembly cap. The transition band and articulation wire assembly can be inserted into the proximal/articulating composite tube thus facilitating assembly.

The deflection section is constructed as a steerable sheath. The steerable sheath construction may provide a larger internal lumen size relative to alternative steerable construction techniques.

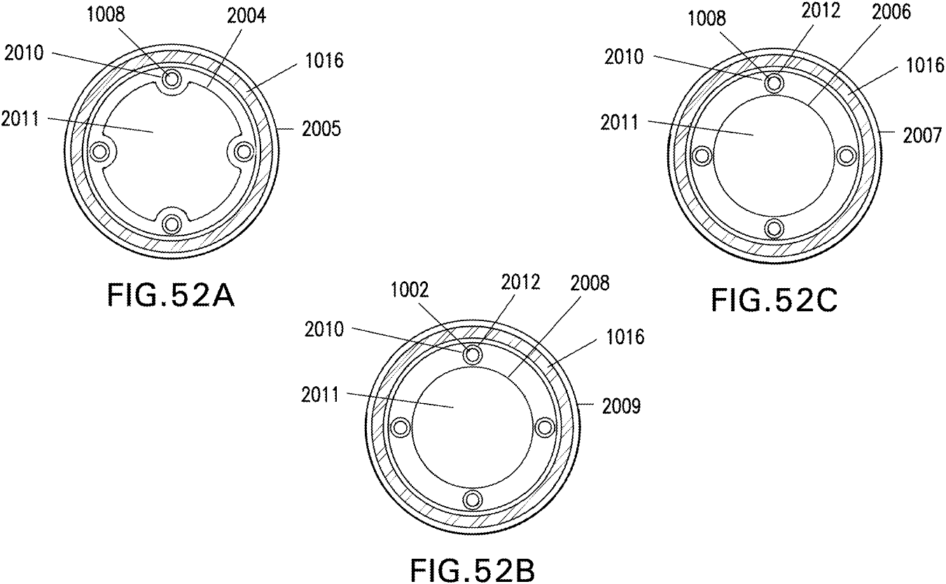

FIG. 52A illustrates a cross sectional view of the proximal shaft section 2001. The proximal shaft includes articulation wire lumens 2010 in which the articulation wires are extended. In some embodiments, there are four articulation wire lumens. Proximal shaft section 2001 further includes a central lumen 2011.

FIG. 52B illustrates a cross sectional view of the deflection section. The deflection section further includes an articulation wire jacket 2012.

FIG. 52C illustrates a cross sectional view of the deflection transition shaft section.

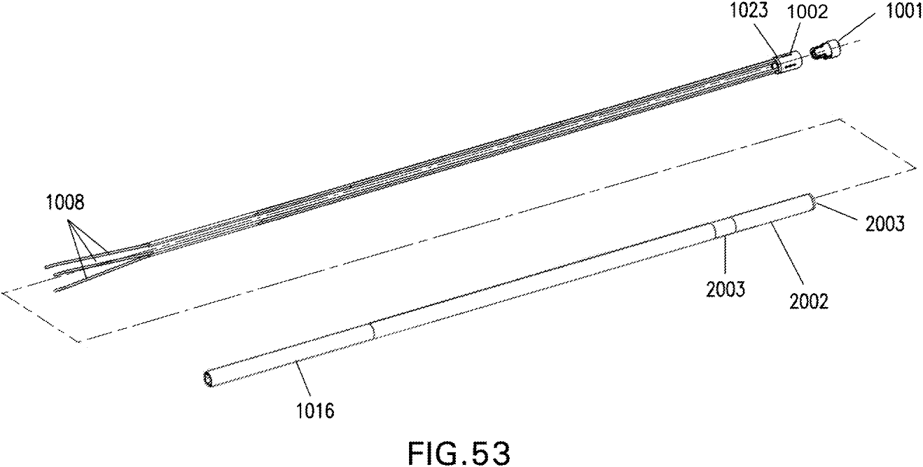

FIG. 53 illustrates a partially exploded view of the insertion tube 2000. As illustrated, during manufacturing of the insertion tube, the articulation components, including the articulation wires, may be pulled as a singular unit into the insertion tube 2000. As illustrated, during manufacturing, the proximal multi lumen shaft section including the articulation wires are extended through the deflection section and pulled through the insertion tube until the articulation wires extend from the end of the insertion tube. By pulling all the interior articulation components as a whole assembly is made easier.

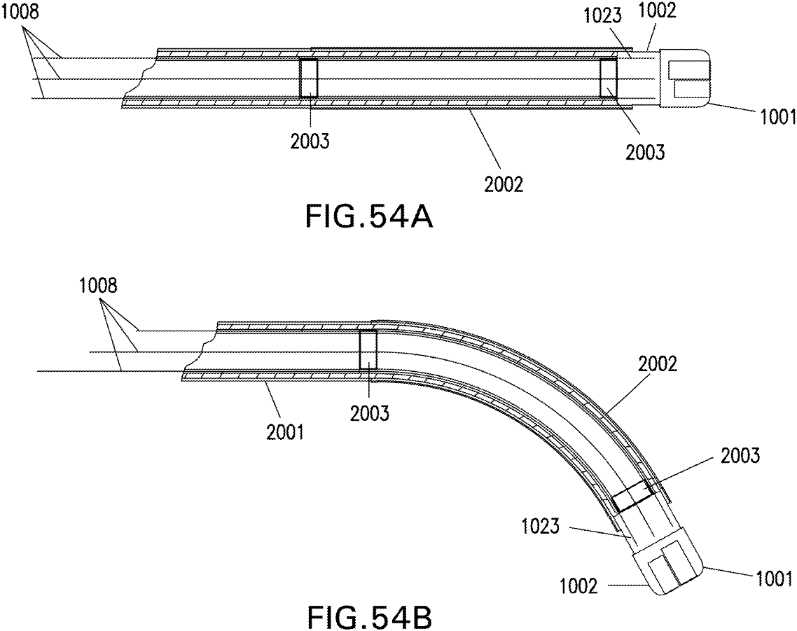

FIG. 54A illustrates the distal end of the insertion tube in a straight configuration. FIG. 54B illustrates the distal end of the insertion tube in an articulated configuration.

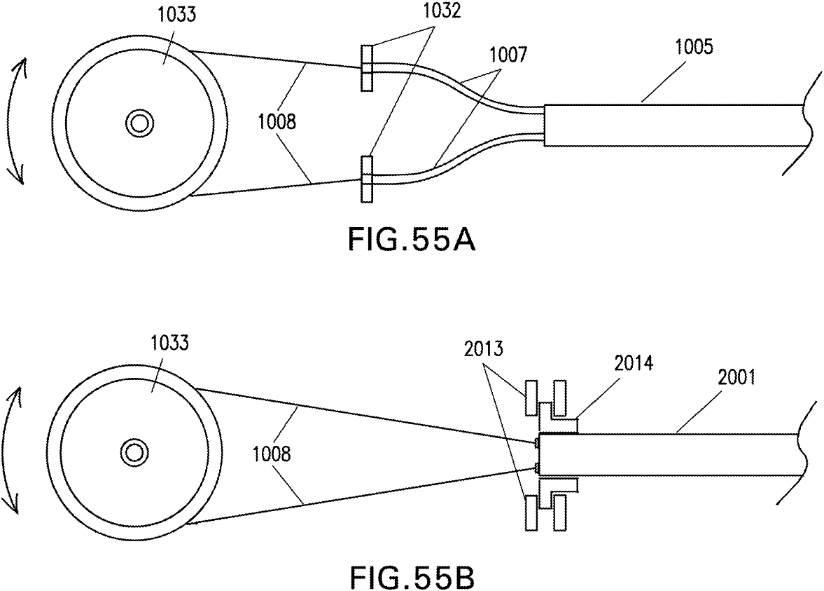

Turning to FIGS. 55-62, a system for articulation wire tensioning with an adjustable stop will be disclosed. The system moves either the compression coil or the catheter shaft distally relative to the proximal pull wire fixation point thus resulting in a tightening of one or more articulation wire.

The use of various systems to adjust the location of the articulation coil or insertion tube relative to the articulation wire are disclosed. Moving the articulation coil or insertion tube relative to the articulation wire provides the needed function of tensioning the articulation control assembly. Moving the articulation coil or insertion tube instead of adjusting the articulation wires has several advantages, including: 1) the articulation wire is more fragile than the articulation coil and, therefore, may be at greater risk of damage during tensioning/repositioning than the articulation coil; 2) if a crimp or adhesive is utilized to secure the articulation wire, it is more difficult to secure and to do so repeatedly than a cut coil/insertion tube; and 3) since the articulation wire typically terminates at a pulley, it is easier to incorporate the tension adjustment into the handle body where the articulating coil terminates.

Turning to FIG. 55A, articulation compression coils 1007 contact an adjustable stop 1032. The stop is arranged to counteract articulation wire force. FIG. 55A further illustrates articulation pulley 1033 with the articulation wires 1008 securely arranged on the pulley. In some embodiments this configuration is used with insertion tube 1000.

Turning to FIG. 55B, insertion tube 2000 contacts an adjustable insertion tube stop 2013. To facilitate cooperation with the stop 2013, the insertion tube includes insertion tube collar 2014.

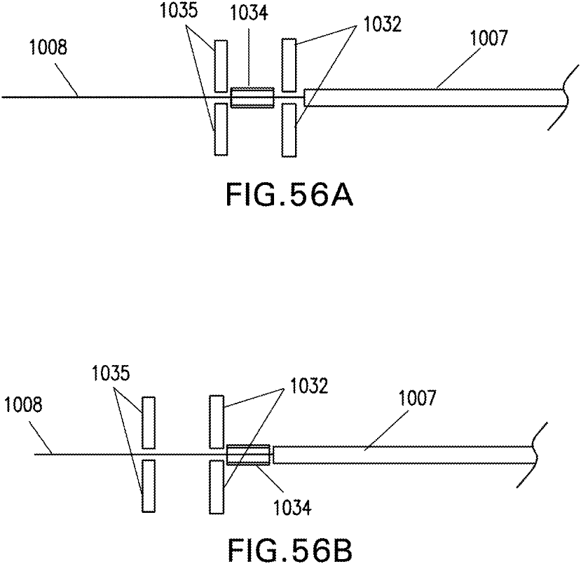

Regarding FIG. 56A and FIG. 56B, structure to adjust stop 1032 is illustrated. In position 1 (FIG. 56A) an adjustable coil spacer 1034 is positioned between stop 1032 and spacer retainer 1035. Spacer retainer is utilized to keep the coil spacer in position against the stop. In a second position (FIG. 56B) coil spacer is position such that stop 1032 is between the coil spacer and the spacer retainer. In this configuration, the coil spacer prevents compression coil 1007 from abutting against stop 1032. The configuration allows the insertion tube to be moved relative to the articulation wire and the handle body to tension the articulation control assembly.

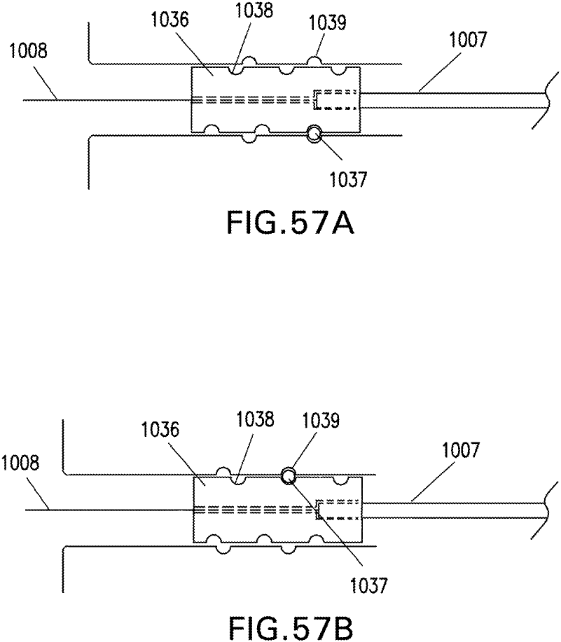

Turning to FIGS. 57A and 57B, a sliding coil retainer 1036 is illustrated with a compression coil 1007 secured within the coil retainer. The compression coil may be secured within the coil retainer by any known mechanism. The coil retainer may include a plurality of grooves 1038 extending along the length of the coil retainer.

The handle body allows for selective repositioning of the coil retainer within the handle body for purposes of tensioning the articulation wires. The handle body includes a plurality of grooves 1039 along the length of the handle body. As the coil retainer is selectively repositioned within the handle body, the grooves 1038 of the coil retainer and the grooves 1039 of the handle body may align. While in alignment, a pin may be inserted within the aligned grooves to lock the coil retainer and thus the compression coil 1007 in place. Other known locking mechanisms may be used to lock the coil retainer relative to the handle body, such as set screws and/or adhesive just to name a few nonlimiting examples. FIG. 57A illustrates the coil retainer in a first position. FIG. 57B illustrates the coil retainer in a second position different from the first position.

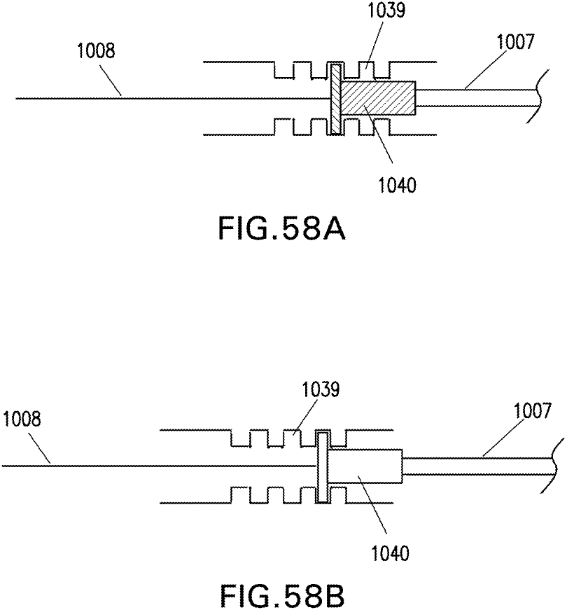

FIGS. 58A and 58B illustrate another embodiment to slide and retain the compression coil 1007. An articulation coil collar 1040 with an upper portion wider than a lower portion of the collar is disclosed. The collar receives and retains the compression coil. In some embodiments, the compression coil is inserted into and/or rotated into the coil collar. The handle body includes a plurality of grooves 1039b along the length of the handle body. The grooves are shaped to receive and retain the upper portion of the collar. FIG. 58A illustrates the stop in a first position. FIG. 58B illustrates the stop in a second position different from the first position.

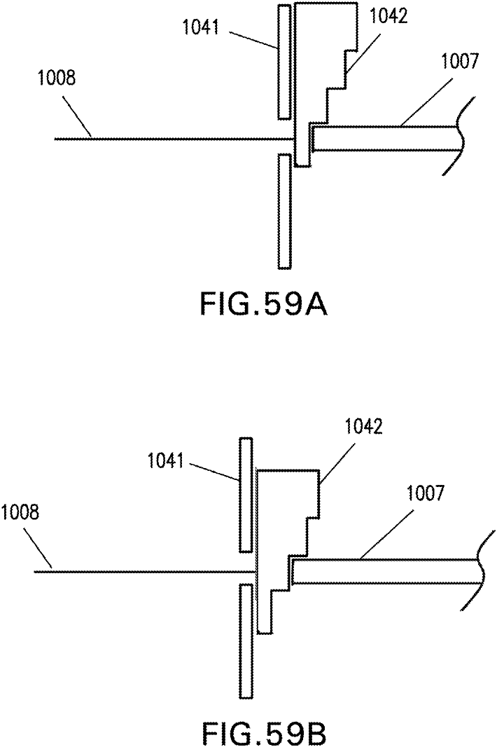

FIGS. 59A and 59B illustrate another embodiment to slide and retain the compression coil 1007. As illustrated, the articulating wire extends through an opening in a portion of the handle 1041. A sliding stepped wedge 1042 is position against the portion of the handle 1041 and covers the opening in which the wire extends through the portion of the handle. The sliding stepped wedge includes a stepped surface with varying heights. The compression coil 1007 may move between steps to change the amount of tensioning on the articulation wire 1008. FIG. 59A illustrates the compression coil on a first step. FIG. 59B illustrates the compression coil on a second step different from the first step.

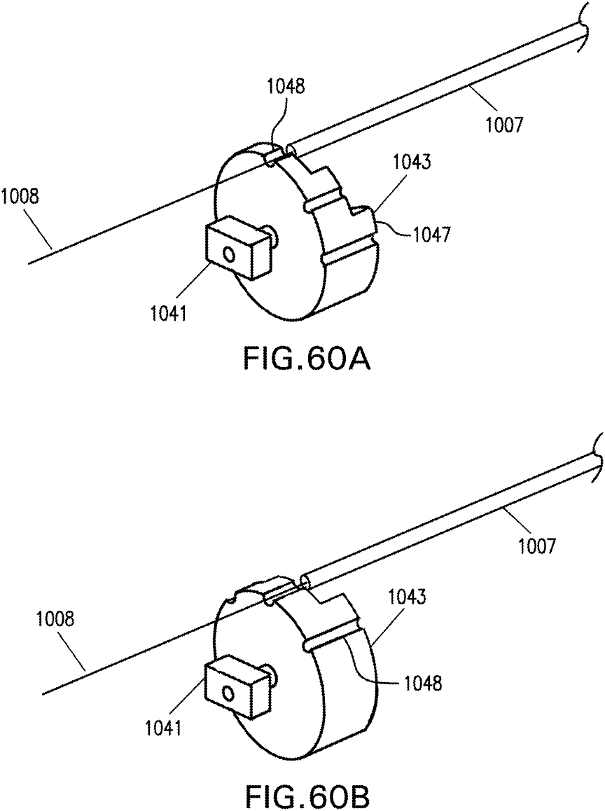

FIGS. 60A and 60B illustrate another embodiment to retain the compression coil 1007. FIGS. 60a and 60b illustrate a rotating stepped wedge 1043. The rotating stepped wedge includes a stepped surface 1047. In some embodiments, the rotating stepped wedge includes 3 steps of various heights. The compression coil abuts and is secured against the stepped surface. Each stepped surface includes a groove 1048 for the articulation wire 1008 to extend through. As the rotating stepped wedge rotates, the compression coil 1007 may move between steps to change the amount of tensioning on the articulation wire 1008. FIG. 60A illustrates the compression coil on a first step. FIG. 60B illustrates the compression coil on a second step different from the first step.

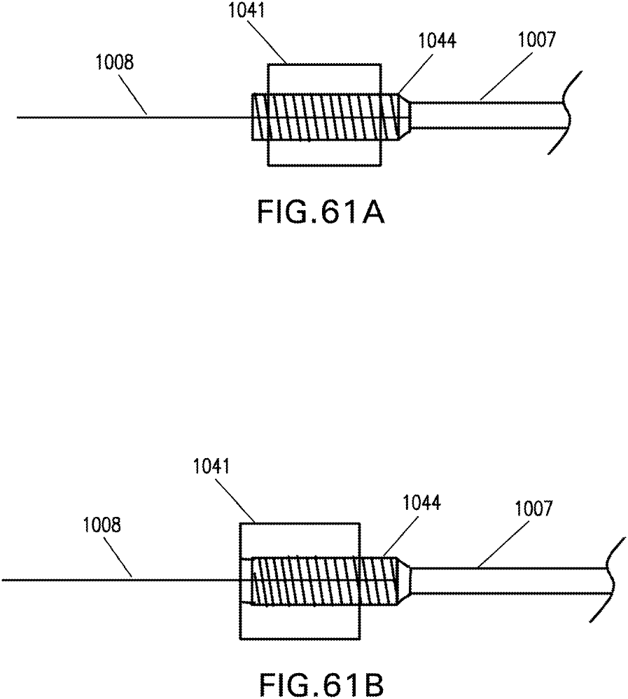

FIGS. 61A and 61B illustrate another embodiment to alter the positioning of the compression coil in order to control tensioning of the articulation wire. This embodiment includes a cannulated set screw 1044 configured against the handle 1041. In this embodiment, the compression coil 1007 is inserted into the set screw and rotated until it is securely retained by the set screw. The compression coil can be rotated within the set screw so that more or less of the compression coil is within the set screw depending on the amount of tensioning necessary. FIG. 61A illustrates the compression coil within the set screw at a first position. FIG. 61B illustrates the compression coil within the set screw at a second position different from the first position.

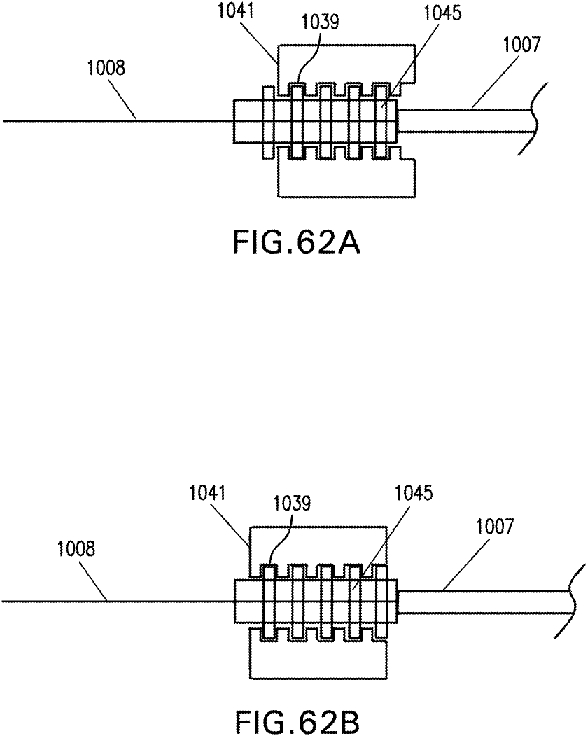

FIGS. 62A and 62B illustrate yet another embodiment to alter the positioning of the compression coil in order to control tensioning of the articulation wire. This embodiment includes a rotating threaded hub 1045 configured to rotate within a plurality of grooves 1039 in the handle. The threaded hub may be rotated to move within the grooves 1039. The compression coil 1007 is inserted and retained within the rotating threaded hub. In order to alter tensioning on the articulation wire, the threaded hub is rotated clockwise or counterclockwise to alter the position of the compression coil in a proximal or distal direction. FIG. 62A illustrates the compression coil and the threaded hub in a first position. FIG. 62B illustrates the compression coil and the threaded hub in a second position different from the first position.

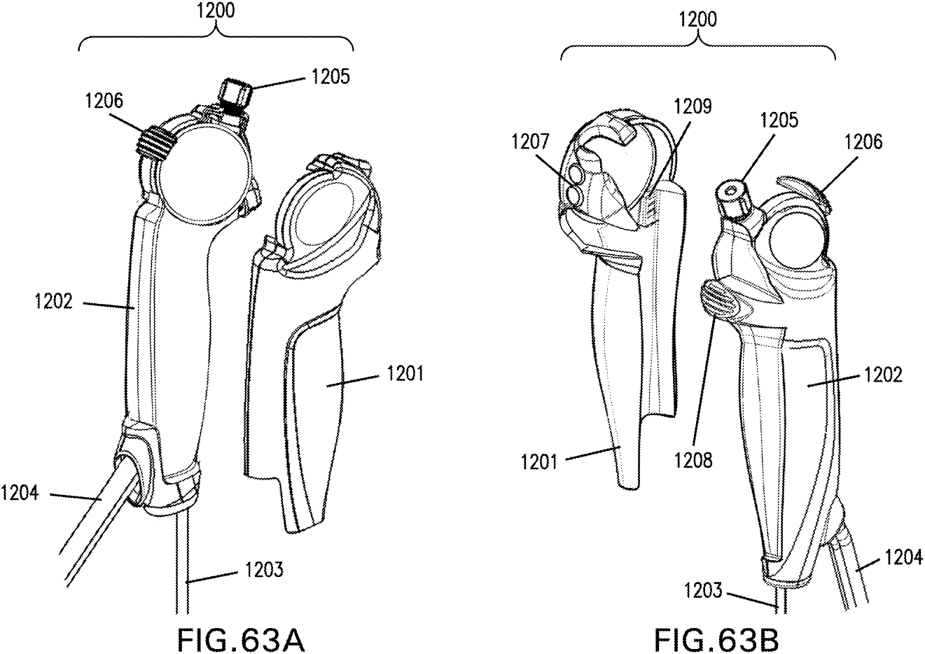

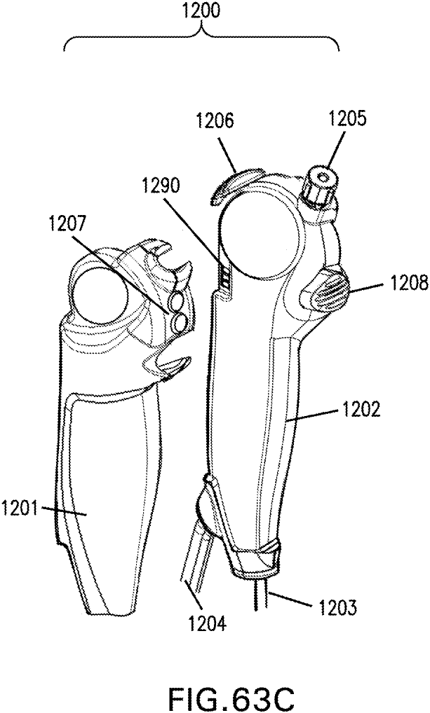

Turning to FIGS. 63-72, a series of hybrid handle (single-use / reusable) variants associated with small diameter two direction (single plane) deflecting endoscopes is disclosed. These hybrid handle variants illustrate varying degrees of functionality associated with the single-use portion of the handle. For example, one hybrid handle variant requires remote fluid control, whereas another variant only incorporates electronic switches for image, illumination, etc. into the reusable handle. The two-direction hybrid handle variants disclosed herein support numerous clinical applications, including: cholangioscopy, ureteroscopy, cystoscopy, and bronchoscopy.

In addition to the hybrid handle variants disclosed, several two-way steering engagement mechanisms are disclosed. These engagement mechanisms involve both self-aligning shaft features and spring-loaded engagement features.

Regarding FIGS. 63A-C, an endoscope assembly 1200 with a separable reusable handle 1201 and a single use endoscope handle 1202 is illustrated. The reusable handle includes electronic control switches 1207 and an electrical conductor 1209. The electronic controls are for image capture, illumination power, video capture, etc.

The single use endoscope handle includes a tool port 1205, insertion tube 1203, umbilical 1204, articulation control lever 1206, fluid control button/trigger 1208 and electrical conductors 1290. The disclosure related to the tool port, various insertion tubes, umbilical, articulation control lever and fluid control button above is incorporated herein. The electrical conductor 1290 connects to electrical conductor 1209 to pass electrical signals between the single use endoscope handle and the reusable handle. The electrical signal from the reusable handle may be passed to the cartridges described above (511 or 800) and passed to a console (600 or 700) when the cartridge (511 or 800) is connected to the console. The electrical signals also pass from the consoles to the distal end of the insertion tube 1203.

The use of the reusable handle reduces waste by moving portions of the endoscope handle structure, mechanism, and electronics away from a single-use, single piece endoscope to a reusable handle in a multi piece endoscope. The relocation of electronic controls from a single-use, single piece endoscope to a reusable handle of a multi piece endoscope, provides for the incorporation of additional features and higher quality components, while not burdening the single-use handle with additional costs. In other words, as opposed to a single use, single piece endoscope that must be thrown away after every use, more expensive and higher quality electronics may be used in the reusable handle and the single-use endoscope handle may be cheaper to manufacturer. Thus, lowering cost by allowing serial usage of the reusable handle with serial single use endoscope handles. The use of electrical conductors 1290 associated with the single-use endoscope handle to provide electrical power and signal connectivity to the reusable handle eliminates the need for secondary cables or batteries that need replacing or charging. The use of a wired connection also eliminates latency and/or connectivity issues often associated with a wireless connection.

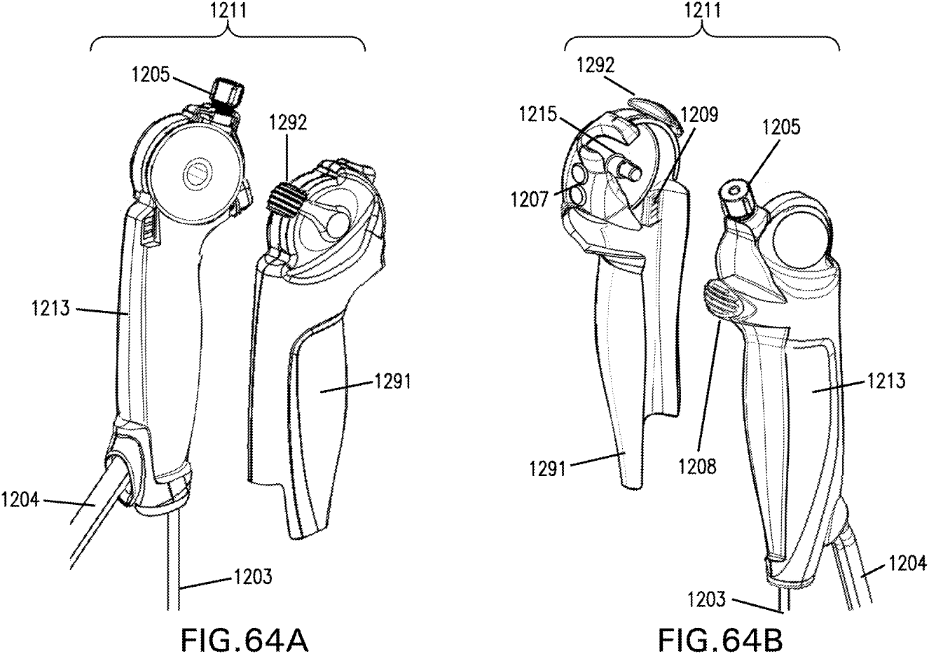

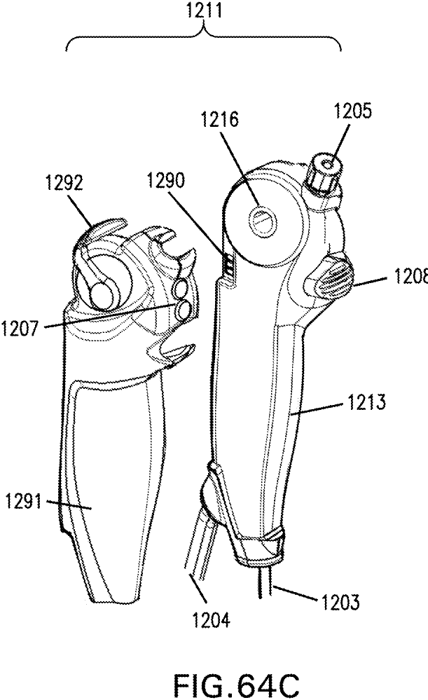

Turning to FIGS. 64A-C, another embodiment endoscope assembly 1211 with a separable reusable handle 1291 and a single use endoscope handle 1213 is illustrated. Many components are similar to the embodiment illustrated in FIGS. 63A-C. Therefore, only the differences between the endoscope assemblies will be discussed with reference to endoscope assembly 1211.

The reusable handle includes electronic controls for image capture, illumination level, video capture, etc. and an articulation lever with optional brake. The reusable handle includes an articulation control lever 1292, similar in function to the articulation control levers disclosed above, and an articulation drive shaft 1215 with engagement features. The single use endoscope handle includes an articulation pulley 1216 with engagement features configured to be compatible with articulation drive shaft 1215. Movement of the lever 1292 will drive the drive shaft 1215 and in turn drive pulley 1216. Pulley 1216 controls the articulation of the articulation wires within the insertion tube 1203.

The use of a self-aligning detachable articulation drive (drive shaft 1215 and pulley 1216) between the reusable handle and the single-use endoscope handle allows for the incorporation of additional features, such as a brake lever/mechanism, and higher quality components, while not burdening the single-use endoscope handle with additional costs. In other words, as opposed to a single use, single piece endoscope that must be thrown away after every use, more expensive and higher quality components may be used in the reusable handle and the single-use endoscope handle will be cheaper to manufacturer. Thus, lowering cost by allowing serial usage of the reusable handle with serial single use endoscope handles.

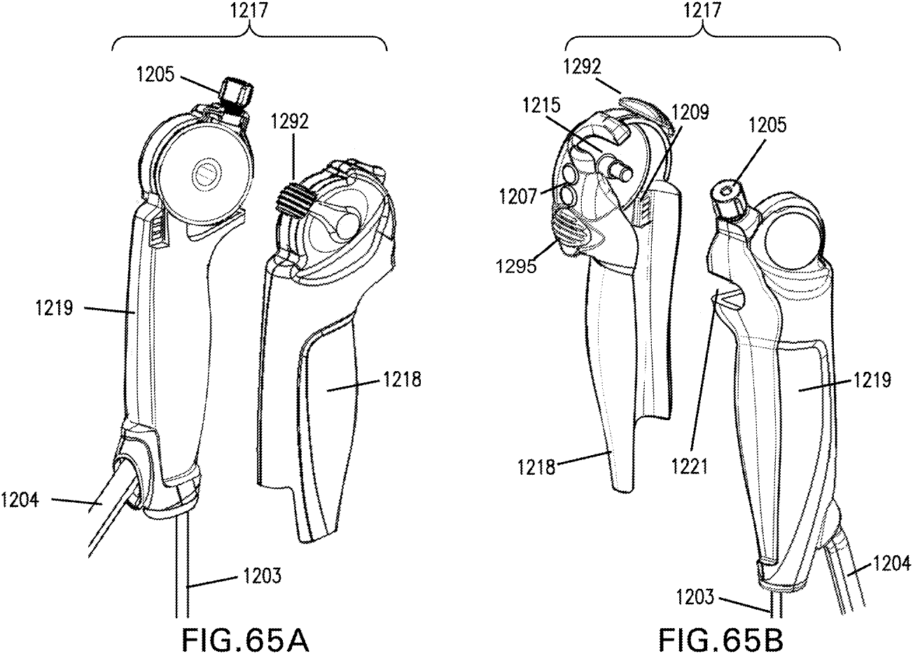

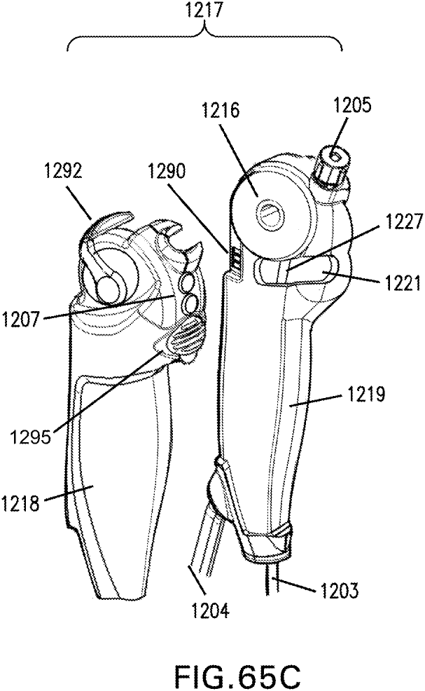

Turning to FIGS. 65A-C, another embodiment endoscope assembly 1217 with a separable reusable handle 1218 and a single use endoscope handle 1219 is illustrated. Many components are similar to the embodiment illustrated in FIGS. 63-64. Therefore, only the differences between the endoscope assemblies will be discussed with reference to endoscope assembly 1217.

The reusable handle includes electronic controls for image capture, illumination level, video capture, etc.; an articulation lever with optional brake; and a fluid button 1295

for mechanically pinching (controlling) a fluid conduit contained within the single use endoscope handle.

The single use endoscope handle includes a slot 1221 and a fluid control tube 1222. When the reusable handle and single use endoscope handle are connected, a pinch valve is created between the fluid control button and the fluid control tube. The fluid control button may be pressed by a user to pinch the fluid control tube to limit the amount of fluid passing through the fluid control tube. The user may control the amount of fluid passing through the fluid control tube by pressing the fluid control button with various force. The fluid control button allows max flow through the tube when the button is not being pressed. The fluid through the tube decreases gradually in relation to the amount the user presses the control button inward.

This embodiment also relocates a mechanical pinch valve button and mechanism from the single-use endoscope handle to the reusable handle. This configuration allows for the incorporation of higher quality components, while not burdening the single-use cartridge with additional costs. In other words, as opposed to a single use, single piece endoscope that must be thrown away after every use, more expensive and higher quality components may be used in the reusable handle and the single-use endoscope handle will be cheaper to manufacturer. Thus, lowering cost by allowing serial usage of the reusable handle with serial single use endoscope handles.

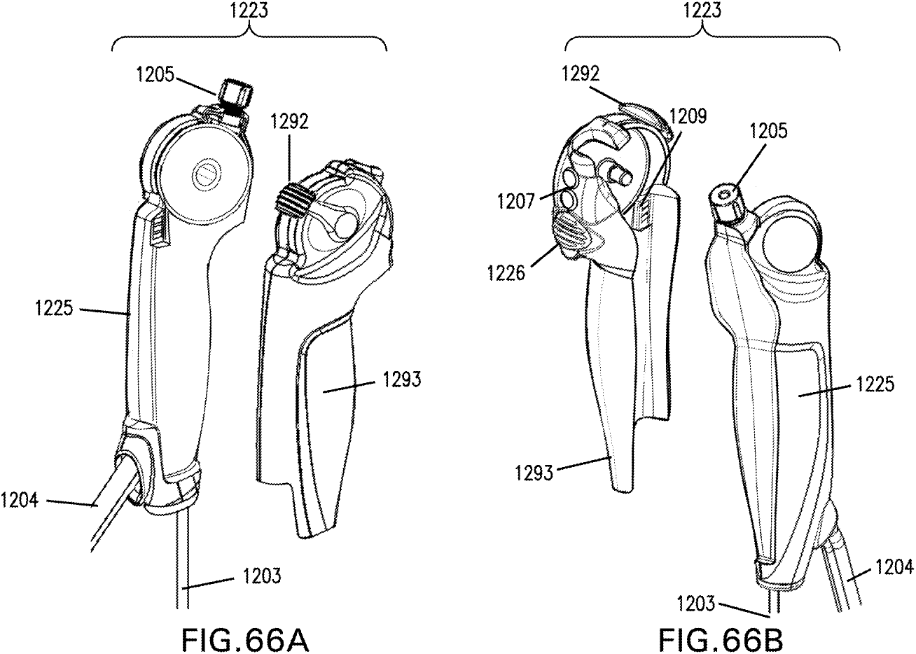

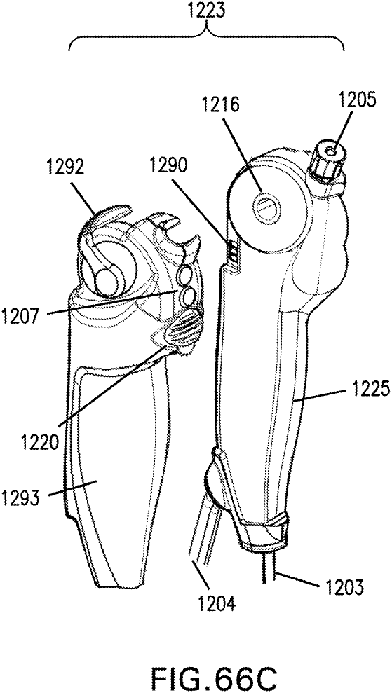

Turning to FIGS. 66A-C, another embodiment endoscope assembly 1223 with a separable reusable handle 1293 and a single use endoscope handle 1225 is illustrated. Many components are similar to the embodiment illustrated in FIGS. 63-65. Therefore, only the differences between the endoscope assemblies will be discussed with reference to endoscope assembly 1223.

The reusable handle includes electronic controls for image capture, illumination level, video capture, etc. and an articulation lever with optional brake. The reusable handle further includes an electrical switch 1226 for pinching (controlling) a fluid conduit using a remotely located electromechanical actuator.

The reusable handle includes electrical switch 1226. When a user presses the electrical switch, a signal is passed to an electromechanical actuator and the actuator actuates onto a fluid path limiting or preventing fluid flow through the fluid path (as described in detail in relation to FIGS. 24 and 29).

This embodiment replaces the mechanical pinch valve button from FIGS. 65a-c and mechanism with the electrical switch 1226 associated with the reusable handle and an electromechanical pinch valve located remote to the endoscope assembly 1223. By removing the mechanical pinch valve from the single-use endoscope handle and/or reusable handle, the cost of the single-use cartridge is reduced.

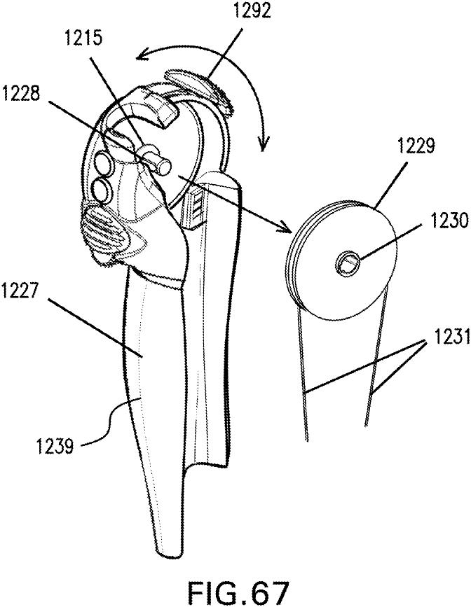

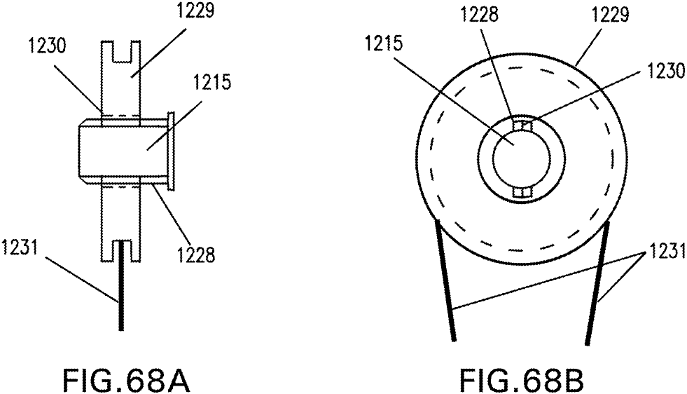

Turning to FIGS. 67 and 68, a self-aligning articulation drive shaft and associated articulation pulley with engagement features are disclosed. As disclosed above, a reusable handle 1227 may include an articulation drive shaft 1215. Drive shaft 215 is driven by articulation control lever 1292 as described above.

In the illustrated embodiment, the housing 1239 of the reusable handle supports drive shaft 1215 connected to the articulation control lever 1292. The drive shaft extends into the region of the reusable handle that receives the single use endoscope handle. The drive shaft of can extend into a recess of the single use endoscope handle such that, when the reusable handle and the single use endoscope handle are joined together, the drive shaft engage the pulley located in the single use endoscope handle.

The drive shaft has a pulley engaging portion 1228. This portion may have a non-circular cross-sectional geometric shape such that it is capable of transmitting torque. This geometric shape can include, but is not limited to, oval, spline, square, or star, just to name a few non-limiting examples.

The single use endoscope handle can include one or more pulleys 1229. The pulley 1229 can be arranged to receive an end of the drive shaft. Preferably, actuation of the articulation control lever associated with the drive shaft rotates the pulley. Preferably, the pulley has an engaging portion 1230 to mate with engaging portion 1228 of the drive shaft 1215 for rotationally coupling the pulley and drive shaft to one another. Preferably, the engaging portions 1228 and 1230 do not rely solely on friction between the drive shaft and pulley. Preferably the mating features include a geometric interference between the articulation shaft and pulley.

Associated with the at least one pulley is an articulation wire 1231 or a pair of articulation wires (e.g., two separate wires or one continuous wire looped over the pulley and having wire segments extending from opposing sides of the pulley) configured to bend the distal end of the insertion tube. The articulation wires may be fixed to the pulley and/or extend around at least a portion of the pulley (e.g., loop around the pulley). Preferably the articulation wires are configured to bend the distal end of the insertion tube in orthogonal planes.

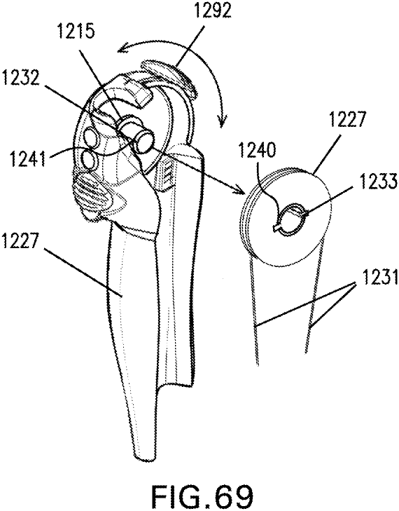

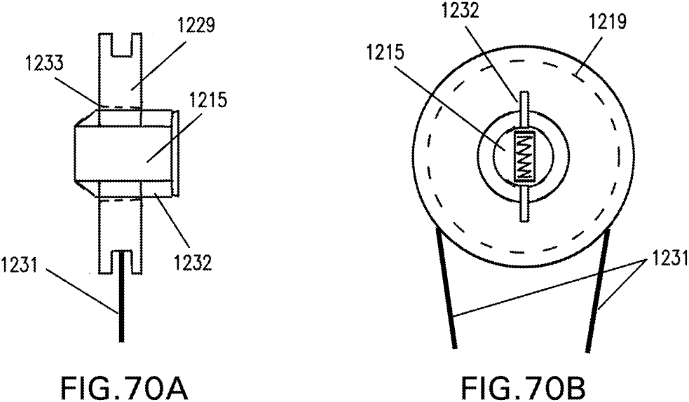

Turning to FIGS. 69 and 70, an alternative embodiment to engage the drive shaft 1215 with the pulley 1229 with spring-loaded engagement features. All applicable discuss above (FIGS. 67-68) is incorporated herein. Drive shaft 1215 may include a spring loaded engagement tab 1232. The spring loaded engagement tab may include one or more spring loaded retractable blades 1241. The spring loaded engagement tab 1232 mates with an engagement feature 1233 on the pulley to operably lock the drive shaft with the pulley. Engagement feature 1233 may include one or more slots 1240 to receive the retractable blades of the spring loaded engagement tab.

The spring loaded retractable blades (with lead-in ramp) allow the user to simply press the two components together. When articulating the control lever through its range of motion, the retracted spring loaded blades will extend and engage the articulation pulley when they encounter the slots on the bore of the articulation pulley. This allows the user to attach the single use endoscope handle without pre-aligning the reusable handle and the single use endoscope handle.

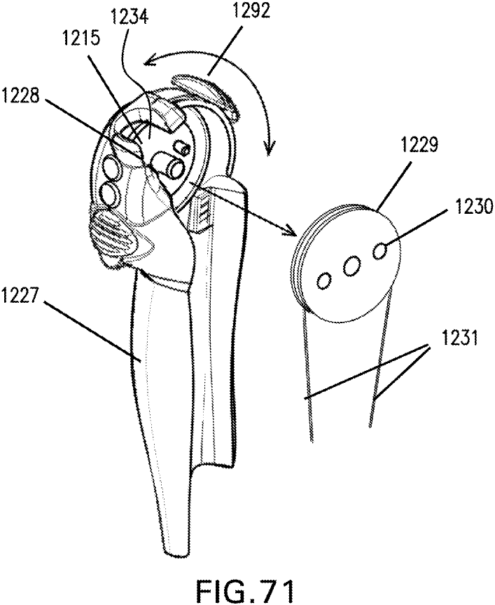

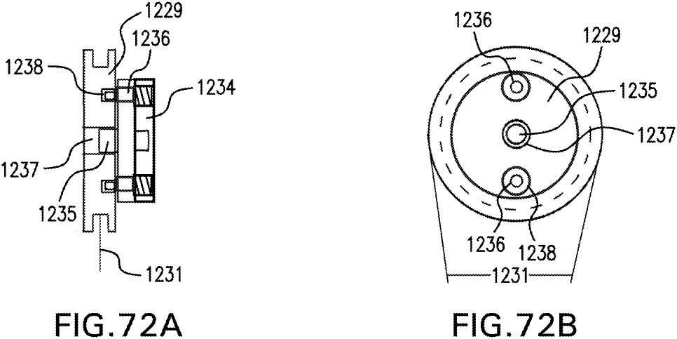

Turning to FIGS. 71 and 72, yet another embodiment is illustrated to mate drive shaft 1215 with pulley 1229. The reusable handle may include a drive disk 1234. Drive disk 1234 may include a center alignment pin 1235, for example an alignment boss with a lead-in chamfer, and one or more spring loaded retractable pins 1236, for example a spring loaded torque pin with a lead-in chamfer, offset from the centerline of the disk to transmit torque. The articulation pulley 1229 may include mating features including a center alignment hole 1237 and offset hole 1238 with a lead-in chamfer with a lead-in for torque pin.

The spring loaded retractable offset pins (with lead-in ramp) allow the user to simply press the two components together. When articulating the control lever through its range of motion, the retracted spring loaded pins will extend and engage the mating features on the face of the articulation pulley. This allows the user to attach the single use endoscope handle without pre-aligning the reusable handle and the single use endoscope handle.

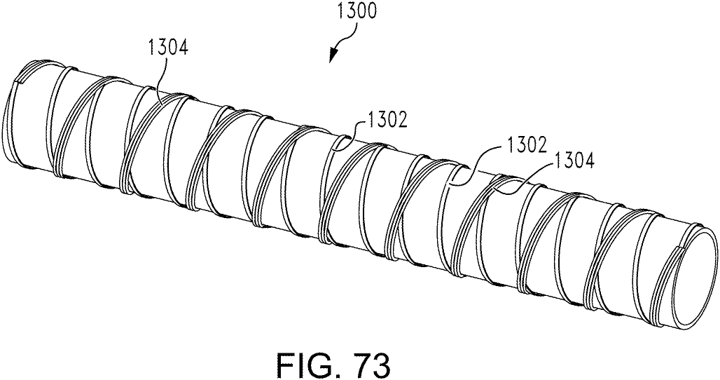

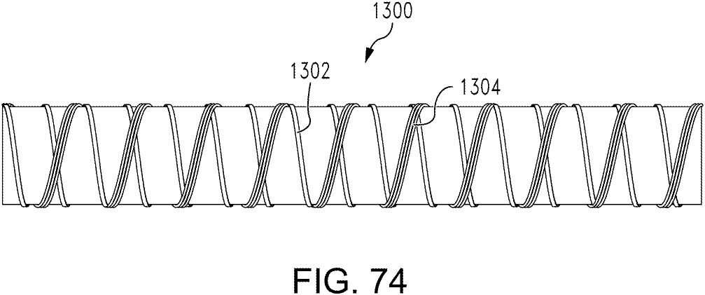

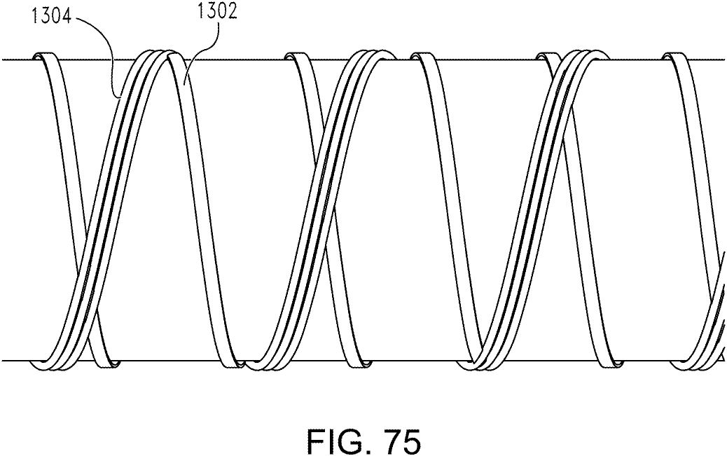

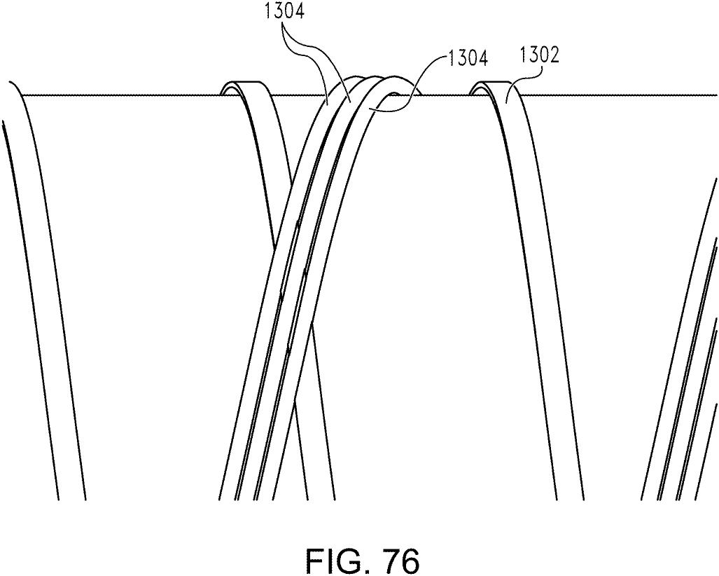

FIGS. 73-76 disclose a novel braid pattern to achieve the mutual benefits of a coil and a braid for an endoscope insertion tube 1300. A novel braid pattern that provides the torqueability associated with a traditional braid while incorporating a unique multi-filar aligned braid pattern in one or more of the helical paths to simulate the kink-resistance associated with a coil is disclosed. This novel improved braid pattern can be created using a traditional braider mechanism with carrier locations modified to generate this novel braid pattern. This novel braid pattern can improve the kink-resistance of small diameter endoscopes, including but not limited to: cholangioscopes, ureteroscopes, cystoscopes, and bronchoscopes.

One embodiment for a small diameter endoscope insertion tube is a single helical filar 1302 over a multi filar group 1304, where the multi filars are all in the same direction. In some embodiments the multi filar group can vary from two to ten or greater. In one exemplary embodiment, the multi filar group is three filars.

The single helical filar 1302 combined with the multi filar group in the opposite direction provide the torqueability associated with a general 1 over 1 braid pattern or other variants thereof.

The multi filar group 1304 are aligned helical patterns, all in the same direction. This configuration creates a simulated coil to provide the insertion tube kink-resistance. The ability to generate a simulated coil during the braiding process reduces process steps, eliminates the need for additional equipment, and eliminates the cost of using a coil when producing an endoscope insertion tube.

While the disclosure has been illustrated and described in detail in the drawings and foregoing description, the same is to be considered as illustrative and not restrictive in character, it being understood that only the preferred embodiment has been shown and described and that all changes, equivalents, and modifications that come within the spirit of the disclosure defined by following claims are desired to be protected. All publications, patents, and patent applications cited in this specification are herein incorporated by reference as if each individual publication, patent, or patent application were specifically and individually indicated to be incorporated by reference and set forth in its entirety herein.

The following numbered clauses set out specific embodiments that may be useful in understanding the present invention:

1. An endoscope, comprising:
an insertion tube assembly and umbilical connected to an elongate handle portion;
the elongate handle portion having an upper end region and a lower end region;
wherein the upper end region of the elongate handle portion includes at least one of:
   an articulation control for articulating a distal portion of an insertion tube assembly connected to the handle portion,
   an irrigation, aspiration, and/or insufflation control, and
   a tool port opening;
wherein the insertion tube assembly and umbilical extend from the lower end region.

2. The endoscope of clause 1, wherein the upper end region of the elongate handle portion includes at least the articulation control, and the irrigation, aspiration, and/or insufflation control.

3. The endoscope of any preceding clause, wherein the upper end region of the elongate handle portion includes at least the articulation control, and the tool port opening.

4. The endoscope of any preceding clause, wherein the upper end region of the elongate handle portion includes at least the irrigation, aspiration, and/or insufflation control and the tool port opening.

5. The endoscope of any preceding clause, wherein the upper end region of the elongate handle portion includes each of the articulation control, the irrigation, aspiration, and/or insufflation control, and the tool port opening.

6. The endoscope of any preceding clause, wherein the irrigation, aspiration, and/or insufflation control is an irrigation control.

7. The endoscope of any preceding clause, wherein the irrigation, aspiration, and/or insufflation control is an aspiration control.

8. The endoscope of any preceding clause, wherein the irrigation, aspiration, and/or insufflation control is an insufflation control.

9. The endoscope of any preceding clause, wherein the irrigation, aspiration, and/or insufflation control comprises a fluid flow adjustment mechanism.

10. The endoscope of clause 9, wherein the fluid flow adjustment mechanism is configured to retain a fluid control button in an at least partially depressed state.

11. The endoscope of clause 9 or 10, wherein the fluid flow adjustment mechanism comprises a rotatable knob.

12. The endoscope of any preceding clause, wherein the irrigation, aspiration, and/or insufflation control comprises a pinch valve positioned within the elongate handle portion configured to compress a flexible fluid tube.

13. The endoscope of any preceding clause, wherein the irrigation, aspiration, and/or insufflation control comprises a normally closed valve positioned within the elongate handle portion.

14. The endoscope of any preceding clause, wherein the irrigation, aspiration, and/or insufflation control controls fluid flow along a first fluid flow path extending through a first lumen;
wherein the first lumen extends through the umbilical;
wherein the first fluid path is in fluid communication with a first tool lumen extending through the insertion tube assembly and in communication with the tool port opening.

15. The endoscope of clause 14, comprising a valve located between the tool port opening and the first fluid path.

16. The endoscope of any one of clauses 14 or 15, wherein a second fluid path extends through a second lumen of the umbilical; and

wherein the second fluid path is in fluid communication with the first tool lumen.

17. The endoscope of any preceding clauses, wherein the articulation control for articulating a distal portion of an insertion tube assembly connected to the handle portion is a lever.

18. The endoscope of clause 17, wherein the lever articulates in a plane that is coextensive with a longitudinal axis of the elongate handle portion.

19. An endoscope, comprising:
a handle portion connected to an insertion tube assembly;
the handle portion include an articulation control for articulating a distal end region of the insertion tube assembly;
wherein the articulation control includes a lever and/or knob connected to a rotatably pulley positioned within the handle portion;
wherein one or more articulation wires extend around the rotatable pulley and through the distal end region of the insertion tube assembly;
wherein a friction element is positioned between the rotatable pulley and a portion of the handle portion so as to resist rotation of the rotatable pulley relative to the housing.

20. The endoscope of clause 19, wherein the friction element is configured to permit articulation of the distal end region of the insertion tube assembly when the articulation control is subjected to a force of 1 Newton or more; and

wherein the friction element is configured to retain the distal end region of the insertion tube assembly in an articulated configuration after removal of the force from the articulation control.

21. The endoscope of clause 19 or 20, wherein the friction element comprises rubber.

22. The endoscope of any one of clauses 19 to 21 wherein the friction element comprises an o-ring.

23. The endoscope of any one of clauses 19 to 22, wherein the friction element comprises friction grease.

24. The endoscope of any one of clauses 19 to 23, wherein the portion of the handle portion extends through the rotatable pulley.

25. A method of assembling an insertion tube for an endoscope, comprising:
advancing one or more fluid and/or tool lumens and electrical conductors through articulating links of a distal portion of the insertion tube along a distal to proximal direction; and
advancing the one or more fluid and/or tool lumens and electrical conductors through a proximal portion of the insertion tube along a distal to proximal direction.

26. The method of clause 25, wherein the one or more fluid and/or tool lumens and electrical conductors are advanced simultaneously through the proximal portion.

27. The method of clause 25 or 26, wherein advancing the one or more fluid and/or tool lumens and electrical conductors through the proximal portion comprises advancing the one or more fluid and/or tool lumens and electrical conductors through a multi-lumen shaft section of the proximal portion.

28. The method of any one of clauses 25 to 27, wherein the one or more fluid and/or tool lumens and electrical conductors are advanced simultaneously through the articulating links.

29. The method of any one of clauses 25 to 28, comprising:
advancing the articulation control wires through the plurality of articulation links; and
securing distal ends of articulation control wires to a distal articulating link and/or termination band.

30. The method of clause 29, wherein advancing the articulation control wires through the plurality of articulation links comprises advancing the articulation control wires through the plurality of articulation links along a distal to proximal direction.

31. The method of clause 29 or 30, comprising:

advancing the articulation controls wires through the proximal portion of the insertion tube along a distal to proximal direction.

32. The method of clause 31, wherein advancing the articulation controls wires through the proximal portion occurs simultaneously with advancing the one or more fluid and/or tool lumens and electrical conductors through the proximal portion.

33. The method of any one of clauses 29 to 32, wherein securing the articulation control wires to the distal articulation link comprises positioning the articulation control wires in grooves of the distal articulation link.

34. The method of any one of clauses 30 to 33, wherein securing the articulation control wires comprises sliding a termination band over the distal termination link while the articulation control wires in grooves of the distal articulation link so as to retain the articulation control wires in the grooves.

35. The method of any one of clauses 29 to 34, wherein securing the articulation control wires to the distal articulating link comprises adhering the articulation control wires to the distal articulation link with adhesive.

36. The method of any one of clauses 29 to 35, wherein securing the articulation control wires to the termination band comprises welding the articulation control wires to the termination band.

37. The method of any one of clauses 25 to 36, wherein the one or more fluid and/or tool lumens comprise a continuous tube or tubes each having a length longer than the sum of lengths of the distal portion and proximal portion.

38. The method of any one of clauses 25 to 37, wherein the one or more electrical conductors comprise continuous wires each having a length longer than the sum of lengths of the distal portion and proximal portion.

39. The method of any one of clauses 25 to 38, wherein the one or more fluid and/or tool lumens and electrical conductors are positioned within a metal coil during advancement through a proximal portion of the insertion tube along a distal to proximal direction.

40. The method of any one of clauses 25to 39, comprising advancing the articulating links of the distal portion of the insertion tube along a distal to proximal direction into a distal portion polymer jacket.

41. The method of clause 40, wherein the proximal portion comprises a proximal portion polymer jacket having a different durometer than said distal portion polymer jacket; and

wherein said distal portion polymer jacket is affixed to a distal end of the proximal portion polymer jacket prior to advancing the articulating links of the distal portion of the insertion tube along a distal to proximal direction into the distal portion polymer jacket

42. The method of any one of clauses 25 to 39, wherein after advancing the one or more fluid and/or tool lumens and electrical conductors through articulating links of a distal portion of the insertion tube along a distal to proximal direction and advancing the one or more fluid and/or tool lumens and electrical conductors through a proximal portion of the insertion tube along a distal to proximal direction;
braiding a wire around the proximal portion of the insertion tube;
reflowing a proximal portion polymer jacket around the proximal portion; and
reflowing a distal portion polymer jacket around the distal portion.

43. The method of clause 42, wherein reflowing the proximal portion polymer jacket around the proximal portion and reflowing the distal portion polymer jacket around the distal portion occur simultaneously.

44. The method of any one of clauses 25 to 39, wherein a distal portion polymer jacket is reflowed around said articulating links of the distal portion of the insertion tube prior to advancing the one or more fluid and/or tool lumens and electrical conductors through the articulating links.

45. A device, comprising:
a first support beam; and
a plurality of articulating links for an endoscope insertion tube coupled to said first support beam through a plurality of support struts;
wherein said articulating links each have a length extending from a first end to a second end;
wherein said articulating links each define a central opening and separate openings for first and second articulation wires extending through said length;
wherein said separate openings for the first and second articulation wires are on opposing sides of the central opening;
wherein the first ends of links of the plurality of articulating links have a convex articulating pivot; and
wherein the second ends of the links have a concave articulating pivot configured to receive the convex pivot of another link.

46. The device of clause 45, wherein the central openings and separate openings of the articulating links extending along a direction transverse to the first support beam.

47. The device of clause 45 or 46, wherein links of the plurality of links are arranged on the support beam in order of position of the articulating links in the endoscope insertion tube.

48. The device of any one of clauses 45 to 47, wherein said plurality of support struts extend from exterior lateral sides of the articulating links.

49. The device of any one of clauses 45 to 48, wherein at least a first articulating link of the plurality of articulating links has a different shape and/or size than a second articulating link of the plurality of articulating links.

50. An endoscope insertion tube assembly, comprising:
a proximal section attachable to a handle of the endoscope;
a distal section comprising a plurality of articulation links;
a transition section positioned between the proximal section and the distal section;
wherein the proximal section comprises polymers having a higher durometer than the transition section; and
wherein the transition section comprises polymers having a higher durometer than the distal section.

51. An endoscope insertion tube assembly, comprising:
a proximal section attachable to a handle of the endoscope; and
a distal section comprising a plurality of articulation links;
wherein a length of the proximal section comprises one or more filars extending helically around a lumen of the proximal section in a first direction; and
wherein the length comprises more filars extending helically around the lumen in a second direction than the one or more filars extending helically around the lumen in the first direction.

52. The assembly of clause 51, wherein at least twice as many filars extend helically around the lumen in the second direction than filars extending helically around the lumen in the first direction.

53. The assembly of clause 51 or 52, wherein only a single filar extends helically around the lumen in the first direction along said length.

54. The assembly of any one of clauses 51 to 53, wherein at least three filars extend helically around the lumen in the second direction along said length.

55. An endoscope insertion tube assembly, comprising:
an articulating joint assembly, the articulation joint assembly having a length extending from a proximal end to a distal end;
   wherein the articulating joint assembly comprises a plurality of articulation links;
wherein each articulation link is connected to an adjacent link and pivotable a pivot angle relative to the adjacent link;
   wherein first and second articulation links of the plurality of articulation links are pivotable to a first side of longitudinal alignment about a first pivot point and pivotable to a second side of longitudinal alignment about a second pivot point; and
   wherein the first and second pivot points are on separate sides for a longitudinal axis extending through said first and second articulation links.

56. The endoscope of clause 55, wherein spacing between the first and second pivot points for pairs of articulation links increases along a length of the endoscope insertion tube assembly.

57. An insertion tube assembly for an endoscope, comprising:
an exterior polymer jacket having an interior surface defining a hollow jacket passage;
a braid positioned within the hollow jacket package, the braid having a hollow interior cross-section defining a braid passage; and
an articulation wire sleeve positioned within the braid passage and between a liner and an interior surface of the braid, the sleeve having a hollow interior cross-section defining a sleeve passage for an articulation wire;
wherein an articulating wire extends through the sleeve and is secured to a termination band positioned distally of the braid.

58. The assembly of clause 57, wherein the sleeve comprises polytetrafluoroethylene.

59. The assembly of clause 57 or 58, wherein the liner comprises polytetrafluoroethylene.

60. The assembly of any one of clauses 57 to 59, wherein the articulating wire extends through the braid in a distal region of the insertion tube.

61. A console for operating and endoscope assembly, comprising:
a receiving area configured to receive a first cartridge of a first endoscope assembly, the receiving area having electrical connectors suitable to form an electrical connection between said console and said first endoscope assembly when the first cartridge is received thereon;
wherein said receiving area is configured to receive a second cartridge of a different size than said first cartridge and form an electrical connection with a second cartridge connected to a second endoscope assembly with said same electrical connectors as with said first cartridge.

62. The console of clause 61, wherein when the second cartridge is received on the receiving area, console actuators in the receiving area do not control fluid flow through second cartridge.

63. An endoscope system, comprising:
a first endoscope having a handle body;
wherein said handle body defines a recess configured to receive a handle body of a second endoscope.

64. The endoscope system of clause 63, wherein the recess includes a mounting structure.

65. The endoscope system of clause 64, wherein the mounting structure comprises:

a strap attached at one end to the handle body and attached at a second end to a latch, and a land for the latch, wherein when the second endoscope is secured to the handle body of the first endoscope, the mounting structure is in a closed position, and wherein in the closed position the latch is operably secured within the land for the latch.

66. The endoscope of clause 65, wherein the latch is a cam latch.

67. The endoscope of clause 65, wherein the mounting structure further comprises:

an alignment pad, wherein the alignment pad matches a profile of the handle body of the second endoscope.

68. An endoscope assembly, comprising
a handle portion and an insertion portion, wherein a proximal end of the insertion portion is attached to the handle portion; and
at least one articulation wire extending through the insertion portion and attached to a pulley within the handle portion;
wherein the tension of the articulation wire is adjustable by at least one of:
repositioning a coil encompassing a portion of the articulation wire relative to the handle portion, and
adjusting an insertion tube of the insertion portion relative to the handle portion.

69. The endoscope assembly of clause 68, wherein the tension of the articulation wire is adjustable by repositioning a coil encompassing a portion of the articulation wire relative to the handle portion.

70. The endoscope assembly of clause 68, wherein the tension of the articulation wire is adjustable by adjusting an insertion tube of the insertion portion relative to the handle portion.

71. The endoscope assembly of clause 68, comprising an adjustable spacer;

wherein the adjustable spacer is configured to retain the coil at any of a variety of positions relative to the handle portion.

72. The endoscope assembly of clause 71, wherein in a first configuration a stop is located between the spacer and the coil to maintain the coil in a first position relative to the housing, and wherein in a second configuration the spacer is between coil and stop to maintain the coil in a second position relative to the housing.

73. The endoscope assembly of clause 68, wherein a retainer is used to secure the coil, wherein the retainer includes at least two grooves along the length of the retainer, wherein the handle portion includes at least two grooves along the length of the handle portion, wherein a pin is inserted between a grove of the retainer and a groove of the handle portion to secure the position of the coil.

74. The endoscope assembly of clause 68, wherein an articulation coil collar is used to secure the coil, wherein the collar includes an upper portion larger than a lower portion of the collar, wherein the handle portion includes at least two grooves for receiving and retaining the upper portion of the collar.

75. The endoscope assembly of clause 68, wherein a stepped wedge with at least two steps is used to secure the position of the coil, wherein on the first step the coil is located in a first position relative to the housing, and wherein on the second step the coil is located in a second position relative to the housing.

76. The endoscope assembly of clause 75, wherein the stepped wedge rotates relative to the housing.

77. The endoscope assembly of clause 75, wherein the stepped wedge slides relative to the housing.

78. The endoscope assembly of clause 68, wherein the coil is threaded and received by a threaded portion of the handle.

79. An endoscope, comprising:
a reusable handle and a single use endoscope handle;
the reusable handle selectively attachable to and detachable from the single use endoscope handle;
wherein the reusable handle includes at least one of:
   electronic conductors and electronic control switches,
   a drive shaft configured to engaged with a pulley located within the single use endoscope handle,
   an articulation control lever,
   a fluid control button for mechanically controlling a fluid conduit contained within the single use endoscope handle, and
   an electrical switch;
wherein actuating the electrical switch actuates an actuator onto a fluid path.

80. The endoscope assembly of clause 79, wherein the reusable handle includes the electronic conductors and the electronic control switches.

81. The endoscope assembly of clause 79, wherein the reusable handle includes the electronic conductors and the electronic control switches and the drive shaft configured to engaged with the pulley located within the single use endoscope handle.

82. The endoscope assembly of clause 79, wherein the reusable handle includes the electronic conductors and the electronic control switches, the drive shaft configured to engaged with the pulley located within the single use endoscope handle, and the articulation control lever.

83. The endoscope assembly of clause 79, wherein the reusable handle includes the electronic conductors and the electronic control switches, the drive shaft configured to engaged with the pulley located within the single use endoscope handle, the articulation control lever, and the fluid control button.

84. The endoscope assembly of clause 79, wherein the reusable handle includes the electronic conductors and the electronic control switches, the drive shaft configured to engaged with the pulley located within the single use endoscope handle, the articulation control lever, and the electrical switch.

85. The endoscope assembly of clause 79, wherein the articulation lever includes a brake.

86. The endoscope assembly of clause 79, wherein the drive shaft includes engagement feature and the pulley includes engagement feature configured to mate with the engagement feature of the drive shaft

87. The endoscope assembly of clause 86, wherein the drive shaft engagement feature comprises a spring loaded engagement tab and the pulley engagement feature comprises a slot configured to mate with the spring loaded engagement tab.

88. The endoscope assembly of clause 87, wherein the spring loaded engagement tab includes at least one retractable blade.

89. The endoscope assembly of clause 86, wherein the drive shaft engagement feature comprises a boss with spring loaded retractable pins and the pulley engagement feature comprises an alignment hole configured to mate with the boss of the drive shaft.

1. An endoscope, comprising:
an insertion tube assembly and umbilical connected to an elongate handle portion;
the elongate handle portion having an upper end region and a lower end region;
wherein the upper end region of the elongate handle portion includes at least one of:
an articulation control for articulating a distal portion of an insertion tube assembly connected to the handle portion,
an irrigation, aspiration, and/or insufflation control, and
a tool port opening; wherein the insertion tube assembly and umbilical extend from the lower end region.

2. The endoscope of claim 1, wherein the upper end region of the elongate handle portion includes at least the articulation control, and the irrigation, aspiration, and/or insufflation control.

3. The endoscope of claim 1, wherein the upper end region of the elongate handle portion includes at least the articulation control, and the tool port opening.

4. The endoscope of claim 1, wherein the upper end region of the elongate handle portion includes at least the irrigation, aspiration, and/or insufflation control and the tool port opening.

5. The endoscope of claim 1, wherein the upper end region of the elongate handle portion includes each of the articulation control, the irrigation, aspiration, and/or insufflation control, and the tool port opening.

6. The endoscope of claim 1, wherein the irrigation, aspiration, and/or insufflation control is an irrigation control.

7. The endoscope of claim 1, wherein the irrigation, aspiration, and/or insufflation control is an aspiration control.

8. The endoscope of claim 1, wherein the irrigation, aspiration, and/or insufflation control is an insufflation control.

9. The endoscope of claim 1, wherein the irrigation, aspiration, and/or insufflation control comprises a fluid flow adjustment mechanism.

10. The endoscope of claim 9, wherein the fluid flow adjustment mechanism is configured to retain a fluid control button in an at least partially depressed state.

11. The endoscope of claim 9, wherein the fluid flow adjustment mechanism comprises a rotatable knob.

12. The endoscope of claim 1, wherein the irrigation, aspiration, and/or insufflation control comprises a pinch valve positioned within the elongate handle portion configured to compress a flexible fluid tube.

13. The endoscope of claim 1, wherein the irrigation, aspiration, and/or insufflation control comprises a normally closed valve positioned within the elongate handle portion.

14. The endoscope of claim 1, wherein the irrigation, aspiration, and/or insufflation control controls fluid flow along a first fluid flow path extending through a first lumen;
wherein the first lumen extends through the umbilical;
wherein the first fluid path is in fluid communication with a first tool lumen extending through the insertion tube assembly and in communication with the tool port opening.

15. The endoscope of claim 14, comprising a valve located between the tool port opening and the first fluid path.

16. The endoscope of claim 14, wherein a second fluid path extends through a second lumen of the umbilical; and
wherein the second fluid path is in fluid communication with the first tool lumen.

17. The endoscope of claim 1, wherein the articulation control for articulating a distal portion of an insertion tube assembly connected to the handle portion is a lever.

18. The endoscope of claim 17, wherein the lever articulates in a plane that is coextensive with a longitudinal axis of the elongate handle portion.

19. An endoscope, comprising:
a handle portion connected to an insertion tube assembly;
the handle portion include an articulation control for articulating a distal end region of the insertion tube assembly;
wherein the articulation control includes a lever and/or knob connected to a rotatably pulley positioned within the handle portion;
wherein one or more articulation wires extend around the rotatable pulley and through the distal end region of the insertion tube assembly;
wherein a friction element is positioned between the rotatable pulley and a portion of the handle portion so as to resist rotation of the rotatable pulley relative to the housing.

20. The endoscope of claim 19, wherein the friction element is configured to permit articulation of the distal end region of the insertion tube assembly when the articulation control is subjected to a force of 1 Newton or more; and
wherein the friction element is configured to retain the distal end region of the insertion tube assembly in an articulated configuration after removal of the force from the articulation control.

21. The endoscope of claim 19, wherein the friction element comprises rubber.

22. The endoscope of claim 19, wherein the friction element comprises an o-ring.

23. The endoscope of claim 19, wherein the friction element comprises friction grease.

24. The endoscope of claim 19, wherein the portion of the handle portion extends through the rotatable pulley.
